(19) United States
(12) Patent Application Publication
(10) Pub. No.: US 2026/0286443 A1
(43) Pub. Date: Sep. 24, 2026

(54) METHODS TO TREAT GLIOMA IN SUBJECTS WITH DEFINED TUMOUR MICROENVIRONMENT

(71) Applicant: UNIVERSITY HEALTH NETWORK, Toronto (CA)

(72) Inventors: Farshad NASSIRI, Etobicoke (CA); Gelareh ZADEH, Etobicoke (ON)

(21) Appl. No.: 19/476,919

(22) PCT Filed: Apr. 18, 2024
(86) PCT No.: PCT/CA2024/050506

§ 371 Date: Oct. 20, 2025

Related U.S. Application Data
(60) Provisional application No. 63/460,794, filed on Apr. 20, 2023.

(51) Int. Cl. C12Q 1/6886; A61K 35/761; A61K 39/00; A61P 35/00; C07K 16/28; C12Q 1/6809

(52) U.S. Cl. CPC C12Q 1/6886; A61K 35/761; A61P 35/00; C07K 16/2818; C12Q 1/6809; A61K 2039/505; A61K 2039/545; C07K 2317/24; C07K 2317/76; C12Q 2600/106; C12Q 2600/158

(57) ABSTRACT

This disclosure relates to methods for identifying a subject with glioma as responsive to combination therapy of an oncolytic virus and an anti-PD-1 antibody, and methods for treating a subject with glioma by determining the tumor microenvironment pretreatment, wherein determining the tumor microenvironment comprises: obtaining a biopsy sample from the subject; determining its immune gene expression profile comprising markers of immune infiltration by detecting the levels of mRNAs of cell type markers, immune checkpoint genes, functional orientation markers, and signature scores; comparing these values to reference samples; determining similarity; and selecting combination therapy if there is a high level of similarity with reference samples with known annotation of medium tumor microenvironment.

### RELATED APPLICATION

This application claims the benefit of U.S. provisional patent application No. 63/460,794 filed on Apr. 20, 2023, herein incorporated by reference.

### INCORPORATION OF SEQUENCE LISTING

A computer readable form of the Sequence Listing “10723-P71573PC00_SequenceListing” (6,217 bytes), filed herewith by electronic submission and created on Apr. 17, 2024, is herein incorporated by reference.

### FIELD

The present disclosure relates to methods of treating glioma in a subject in need thereof. In particular, the method relates to identifying a subject as responsive to combination therapy of an oncolytic virus and an anti-PD-1 antibody by determining the tumor microenvironment pretreatment.

### BACKGROUND

Glioblastoma is the most common and lethal adult primary brain tumor. The standard of care treatment for newly diagnosed patients includes surgical resection followed by concomitant chemoradiotherapy and adjuvant temozolomide¹. Despite maximal multimodal therapy, patients invariably experience recurrence of their disease 7 months after diagnosis, on average¹. Unfortunately, treatment options at recurrence are scarce. Existing salvage therapies have very limited efficacy, with median survival being in the range of only 6-8 months after tumor progression². Effective treatments for recurrent disease are urgently needed.

While immune checkpoint blockade by anti-PD-1 or anti-PD-L1 antibodies have improved outcomes with objective responses in a variety of other cancers, including those in the brain such as metastatic melanoma³, they have had limited efficacy as monotherapy for recurrent glioblastoma where the microenvironment is innately immunosuppressive (i.e., immunologically ‘cold’)^{4,5}. Oncolytic viruses are capable of reconditioning the tumor microenvironment towards a ‘hot’ phenotype, providing rationale for combinatorial therapy with checkpoint inhibitors, which has been shown to improve outcomes in other cancers^{6,7}.

DNX-2401 (tasadenoturev; Delta-24-RGD) is a conditionally replicative oncolytic adenovirus engineered to treat high-grade malignant gliomas^{8,9}. The virus contains two stable genetic changes in the adenovirus dsDNA genome that cause it to selectively and efficiently replicate in cancerous cells. A dose escalation phase 1 study demonstrated that stereotactic delivery of DNX-2401 into patients with high-grade gliomas was safe and induced cell death initially by direct oncolysis and subsequently by antitumor response from infiltrated immune cells, with durable responses after a single intratumoral dose¹⁰.

### SUMMARY

The results of CAPTIVE (2401BT-002P; KEYNOTE-192; NCT02798406), a 2-part, phase 1/2, multicenter, open-label clinical trial of combined intratumoral injection of DNX-2401 with systemic pembrolizumab for patients with recurrent glioblastoma is herein disclosed. This is the first in-human investigation of combined oncolytic virus with immune checkpoint blockade for recurrent glioblastoma.

The present inventors determined that tumors with a tumor microenvironment characterized as having a medium degree of immune cell enrichment responded to the combination therapy of an oncolytic virus, DNX-2401, with an anti-PD-1 antibody, pembrolizumab, better than tumors with a low or high degree of immune cell enrichment.

Accordingly, in one aspect, provided herein is a method of selecting therapy for a subject with glioma comprising:
(a) obtaining a biopsy sample from the subject;
(b) determining a sample immune gene expression profile comprising markers of immune infiltration by detecting the levels of mRNAs of cell type markers, immune checkpoint genes, functional orientation markers, and signature scores;
(c) comparing the values of b) with a control profile comprising gene expression values from reference samples with known annotation of high (TMEhigh), medium (TMEmedium) and low (TMElow) tumor microenvironments;
(d) determining the level of similarity of b) to c), and
(e) selecting combination therapy of an oncolytic virus and an anti-PD-1 antibody or a binding fragment thereof if there is a high level of similarity of b) to TMEmedium; a low level of similarity of b) to TMEhigh or TMElow; and/or a higher level of similarity of b) to TMEmedium than to TMEhigh or TMElow, and not selecting combination therapy of an oncolytic virus and an anti-PD-1 antibody or a binding fragment thereof if there is a low level of similarity of b) to TMEmedium; a high level of similarity of b) to TMEhigh or TMElow; and/or a higher level of similarity of b) to TMEhigh or TMElow than to TMEmedium.

In another aspect, herein provided is a method of treating a subject with glioma comprising:
(a) obtaining a biopsy sample from the subject;
(b) determining a sample immune gene expression profile comprising markers of immune infiltration by detecting the levels of mRNAs of cell type markers, immune checkpoint genes, functional orientation markers, and signature scores;
(c) comparing the values of b) with a control profile comprising gene expression values from reference samples with known annotation of high (TMEhigh), medium (TMEmedium) and low (TMElow) tumor microenvironments;
(d) determining the level of similarity of b) to c); and
(e) administering combination therapy of an oncolytic virus and an anti-PD-1 antibody or a binding fragment thereof to the subject if there is a high level of similarity of b) to TMEmedium; a low level of similarity of b) to TMEhigh or TMElow; and/or a higher level of similarity of b) to TMEmedium than to TMEhigh or TMElow; and not administering combination therapy of an oncolytic virus and an anti-PD-1 antibody or a binding fragment thereof to the subject if there is a low level of similarity of b) to TMEmedium; a high level of similarity to TMEhigh or TMElow; and/or a higher level of similarity of b) to TMEhigh or TME low than to TMEmedium.

In an embodiment, determining a higher level of similarity is indicated by a higher correlation value computed between the immune gene expression profile and the control profile, optionally wherein the correlation value is a correlation coefficient. In another embodiment, determining a lower level of similarity is indicated by a lower correlation value computed between the sample immune gene expression profile and control profile, optionally wherein the correlation value is a correlation coefficient. In one embodiment, the correlation coefficient is a linear coefficient, optionally a Pearson correlation coefficient or a Spearman correlation coefficient.

In another embodiment, a high level of similarity is indicated by a Pearson correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In yet another aspect, herein provided is a method of treating glioma comprising administering combined therapy of an oncolytic virus and anti-PD-1 antibody or a binding fragment thereof to a subject that has been previously identified as having a medium tumor microenvironment,
wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

In yet another aspect, herein provided is a use of a combination therapy of an oncolytic virus and an anti-PD-1 antibody or a binding fragment thereof for treating glioma in a subject that has been previously identified as having a medium tumor microenvironment,
wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

In yet another aspect, herein provided is a combination therapy of an oncolytic virus and an anti-PD-1 antibody or a binding fragment thereof for use in treating glioma in a subject that has been previously identified as having a medium tumor microenvironment,
wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

In yet another aspect, herein provided is a use of a combination therapy of an oncolytic virus and an anti-PD-1 antibody or a binding fragment thereof in the manufacture of a medicament for treating glioma in a subject that has been previously identified as having a medium tumor microenvironment,
wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

In some embodiments, the cell type markers comprise B-cells, CD45, CD8 T cells, Cytotoxic cells, Dendritic cells, exhausted CD8 cells, macrophages, neutrophils, NK CD56dim cells, NK cells, T-cells, Gh1 cells and Treg cells.

In an embodiment, the B-cell markers comprise BLK, CD19, MS4A1 and TNFRSF17.

In an embodiment, the CD45 marker comprises PTPRC.

In an embodiment, the CD8 T cell markers comprise CD8A and CD8B.

In an embodiment, the cytotoxic cell markers comprise CTSW, GNLY, GZMA, GZMB, GZMH, KLRB1, KLRD1, KLRK1 and PRF1.

In an embodiment, the dendritic cell markers comprise CCL13, CD209 and HSD11B1.

In an embodiment, the exhausted CD8 cell markers comprise CD244, EOMES and LAG3.

In an embodiment, the macrophage markers comprise CD163, CD68 and CD84.

In an embodiment, the mast cell markers comprise MS4A2 and TPSAB1/B2.

In an embodiment, the neutrophil cell markers comprise CSF3R, FCGR3A/B and S100A12.

In an embodiment, the NKCD56 dim cell markers comprise IL21R, KIR2DL3, KIR3DL1 and KIR3DL2.

In an embodiment, the NK cell marker comprises NCR1.

In an embodiment, the T-cell marker comprises CD3D, CD3E, CD3G, CD6 and SH2D1A.

In an embodiment, the Th1 cell marker comprises TBX21.

In an embodiment, the Treg cell marker comprises FOXP3.

In an embodiment, the immune checkpoint genes comprise PD-1 (PDCD1), PD-L1 (CD274), PD-L2 (PDCDILG2), CTLA4, TIM3, LAG3, TIGIT, B7-H3, IDO1, ICOS, NOS2, ARG2 and CXCL9.

In some embodiments, the functional orientation markers comprise T cell activation, T cell inhibition, Class 1 MHC, regulatory T cells, Myeloid cell chemotaxis, MI markers, M2 markers, Tertiary lymphoid structures, angiogenic markers.

In an embodiment, the T cell activation markers comprise CD70, CD244, CD48 and CD44.

In an embodiment, the T cell inhibition markers comprise LAG3, TNFRSF8, CTLA4, TIGIT, PDCDI, HAVCR2, BTLA, ADORA2A, TNFRSF25 and LAIR1.

In an embodiment, the regulatory T cell markers comprise FOXP3 and TNFRSF18.

In an embodiment, the Class 1 MHC markers comprise HLA-A, HLA-B, HLA-C, HLA-E, HLA-F and B2M.

In an embodiment, the myeloid cell chemotaxis markers comprise CCL2, CCL5, VEGFA and CSF1.

In an embodiment, the M1 markers comprise CCL2, CXCL10, GBP2, IFIT3, SLAMF7, CXCL9, CCL8, IL1B and CD38.

In an embodiment, the M2 markers comprise CXCL16, CXCR4, CD14, MRC1, ARG1 and CCL13.

In an embodiment, the tertiary lymphoid structure markers comprise CCL2, CCL4, CCL5, CCL8, CCL18, CCL19, CCL21, CXCL9, CXCL10, CXCL11 and CXCL13.

In an embodiment, the angiogenic markers comprise VEGFA, VEGFB, KDR, CXCR2, HIFIA and ANGPT2.

In some embodiments, the signature scores comprise chemokine, cytolytic, interferon gamma signaling, interferon gamma downstream signals and T cell inflamed.

In an embodiment, the chemokine markers comprise CCL2, CCL4, CCL5, CCL8, CCL18, CCL19, CCL21, CXCL9, CXCL10, CXCL11 and CXCL13.

In an embodiment, the cytolytic markers comprise GZMA and PRF1.

In an embodiment, the interferon signaling markers comprise IDO1, CXCL10, CXCL9, HLA-DRA, STAT1 and IFNG.

In an embodiment, the interferon gamma downstream signal markers comprise CD3D, IDO1, CD3E, CCL5, GZMK, CD2, HLA-DRA, CXCL13, IL2RG, NKG7, HLA-E, CXCR6, LAG3, CXCL10, STAT1 and GZMB.

In an embodiment, the T cell inflamed markers comprise CCL5, CD27, CD274 [PD-L1], CD276 [B7-H3], CD8A, CMKLR1, CXCL9, CXCR6, HLA.DQA1, HLA.DRB1, HLA.E, IDO1, LAG3, NKG7, PDCDILG2 [PD-L2], PSMB10, STAT1, and TIGIT.

In some embodiments, testing the sample for cell type markers, immune checkpoint genes, functional orientation markers, and signature scores comprises measuring gene expression level of ADORA2A, ANGPT2, ARG1, B2M, BLK, BTLA, CCL13, CCL18, CCL19, CCL2, CCL21, CCL4, CCL5, CCL8, CD14, CD163, CD19, CD2, CD209, CD244, CD38, CD3D, CD3E, CD3G, CD44, CD48, CD6, CD68, CD70, CD84, CD8A, CD8B, CSF1, CSF3R, CTLA4, CTSW, CXCL10, CXCL11, CXCL13, CXCL16, CXCL9, CXCR2, CXCR4, CXCR6, EOMES, FCGR3A/B, FOXP3, GBP2, GNLY, GZMA, GZMB, GZMH, GZMK, HAVCR2, HIFIA, HLA-A, HLA-B, HLA-C, HLA-DRA, HLA-E, HLA-F, HSD11B1, IDO1, IFIT3, IFNG, IL1B, IL21R, IL2RG, KDR, KIR2DL3, KIR3DL1, KIR3DL2, KLRB1, KLRD1, KLRK1, LAG3, LAIR1, MRC1, MS4A1, MS4A2, NCR1, NKG7, PDCD1, PRF1, PTPRC, S100A12, SH2DIA, SLAMF7, STAT1, TBX21, TIGIT, TNFRSF17, TNFRSF18, TNFRSF25, TNFRSF8, TPSAB1/B2, VEGFA, VEGFB.

In some embodiments, the glioma is glioblastoma.

In some embodiments, the oncolytic virus is administered intratumorally prior to repeated doses of anti-PD-1 antibody or a binding fragment thereof.

In some embodiments, the repeated doses of anti-PD-1 antibody or a binding fragment thereof start about 7 days after administration of the oncolytic virus.

In some embodiments, the doses of anti-PD-1 antibody or a binding fragment thereof are administered as about 200 mg infused intravenously over about 30 minutes.

In some embodiments, the anti-PD-1 antibody or the binding fragment thereof is pembrolizumab or a fragment thereof.

In some embodiments, the oncolytic virus is an oncolytic adenovirus.

In some embodiments, the oncolytic adenovirus is a conditionally replicative oncolytic adenovirus.

In some embodiments, the conditionally replicative oncolytic adenovirus is DNX-2401.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF DRAWINGS

The embodiments of the disclosure will now be described in greater detail with reference to the attached drawings in which:

FIG. 1a shows patient flow in an exemplary embodiment of the disclosure. Patient flow in trial.

FIG. 1b shows the maximal change in tumor size in an exemplary embodiment of the disclosure. Waterfall plot that displays the maximal change in tumor size for all patients who received full-dose DNX-2401 treatment (n=42). Bars represent the maximal tumor change from baseline on the basis of contrast-enhanced magnetic resonance imaging. Bars are shaded according to responses classified according to modified Response Assessment in Neuro-Oncology (mRANO) criteria.

FIG. 1c shows the survival of patients by DNX-2401 dose in an exemplary embodiment of the disclosure. Survival for each patient by DNX-2401 dose. The bars are shaded according to response to treatment by mRANO criteria. Arrows indicate that the patient remains alive.

FIG. 1d shows overall survival for the intent to treat population in an exemplary embodiment of the disclosure. Crosses denote censored data. Median overall survival (denoted by the vertical dashed line) was 12.5 months, with a 95% confidence interval ranging from 10.7-13.5 months.

FIG. 2a shows complete responses to DNX-2401 and Pembrolizumab in an exemplary embodiment of the disclosure. Axial Tl-Weighted MR (top row) and Fluid-attenuated inversion recovery (FLAIR) images (bottom row) obtained at baseline, 3 months, 6 months, 12 months, and 38 months after infusion of DNX-2401 for one complete responder.

FIG. 2b shows the change of tumor size over time in each patient with a complete response in an exemplary embodiment of the disclosure. Bottom dashed line represents the threshold for response according to the modified Response Assessment in Neuro-Oncology criteria. Both patients showed response to treatment at 3 months after DNX-2401 infusion, with complete response by 12-14 months.

FIG. 3a shows changes in perilesional FLAIR signal hyperintensity at each study MRI time point for individual patients in an exemplary embodiment of the disclosure. Patients are stratified according to their mRANO responses.

FIG. 3b changes in perilesional FLAIR signal hyperintensity at each study time point relative to baseline MRI in an exemplary embodiment of the disclosure. Patients are stratified by whether they developed clinically relevant edema requiring medical treatment.

FIG. 4a shows survival of patients in an exemplary embodiment of the disclosure. Kaplan-Meier survival curve with patients stratified by response to treatment classified using mRANO. Crosses denote censored data. Median overall survival (OS) for Partial Response (denoted by the right-most vertical dashed line) was 21.5 months, with a 95% confidence interval ranging from 15.5 months at the lower limit to no upper limit. Median OS for Progressive Disease (denoted by the left-most vertical dashed line) was 10.8 months, with a 95% confidence interval ranging from 7.9-14.6 months. Median OS for Stable Disease (denoted by the middle vertical dashed line) was 12.5 months, with a 95% confidence interval ranging from 10.8-16.9 months.

FIG. 4b shows survival of patients in an exemplary embodiment of the disclosure. Kaplan-Meier survival curve and associated 95% CI using landmark 6-months method with patients stratified according to objective response status. Crosses denote censored data. Patients with objective responses (Yes; complete or partial responses by mRANO) had statistically longer survival than those without objective responses (No; stable or progressive disease by mRANO, HR 0.20, 95% CI 0.05 to 0.87, log rank test P=0.02). Median overall survival (OS) for Yes (denoted by the right-most vertical dashed line) was 21.5 months, with a 95% confidence interval ranging from 15.5 months at the lower limit to no upper limit. Median OS for No (denoted by the left-most vertical dashed line) was 12.7 months, with a 95% confidence interval ranging from 11.5-14.7 months.

FIG. 5a shows mRNA expression prior to treatment in an exemplary embodiment of the disclosure. Heatmap showing three subtypes of glioblastoma microenvironment in samples from this trial on the basis of enrichment for immune cell types using partition around medoids clustering. Scores for functional orientation markers, signature scores, and expression of immune checkpoints and biomarkers are overlayed on the heatmap.

FIG. 5b show the distribution of expression of PD-1 (PDCD-1; left) and PD-L1 (CD274; right) across different tumor size comparisons in an exemplary embodiment of the disclosure.

FIG. 5c shows the rate of clinical benefit in an exemplary embodiment of the disclosure. Stacked barplot showing the rate of clinical benefit (stable disease or objective response) stratified by microenvironment subtypes in this study (left) and previously published cohort examining adjuvant PD-1 monotherapy in recurrent glioblastoma (right). Dashed box represents the proportion of objective responses by mRANO criteria in this study.

FIG. 5d shows the distribution of overall survival of patients in an exemplary embodiment of the disclosure. Distribution of overall survival of patients in this trial (left; log-rank test P=0.03, median OS for TME-low=11.5 months, median OS for TME-medium=15.5 months, and median OS for TME-high=10.7 months) and previously published trial (right; log-rank test P=0.86) stratified by immune microenvironment subtypes.

FIG. 5e shows three subtypes of glioblastoma microenvironment in an exemplary embodiment of the disclosure. Heatmap showing three subtypes of glioblastoma microenvironment on previously published cohort examining adjuvant PD-1 monotherapy in recurrent glioblastoma.

FIG. 6 shows distribution of scores in an exemplary embodiment of the disclosure for immune cell types (top row), immune checkpoint genes and biomarkers (second row), functional orientation markers (third row) and signature scores (last row). Shown are boxplots for each TME subtype. Dots denote individual values. Central bars indicate medians, the box defines the upper and lower quartiles of the distribution, and whiskers define the 1.5×IQR. All comparisons have p<0.05 unless specifically indicated with ns (P>0.05).

FIG. 7a shows comparison of gene mRNA expression at disease progression to baseline in an exemplary embodiment of the disclosure. Volcano plot showing the expression changes of individual genes after treatment compared to baseline. Genes that are differentially expressed (absolute Log₂FC greater than 1 and P-value less than 0.05) are labelled.

FIG. 7b shows a barplot showing results of functional enrichment analysis of differentially expressed genes in FIG. 5a in an exemplary embodiment of the disclosure.

FIG. 7c shows differences in gene expression profiles and signatures in patients in an exemplary embodiment of the disclosure. Forest plot summarizing differences in gene expression profiles and signatures for 9 patients who did not have objective response to treatment (shown in black) with expression data at both disease progression and baseline. Dot denotes average and bars represent 95% CI. Light grey dot denotes one additional patient with objective partial response to treatment. FC denotes fold change.

FIG. 8a shows immunophenotyping in an exemplary embodiment of the disclosure. Representative immunohistochemical stains for microglia (Iba1), macrophages (CD68), and lymphocytes (CD3, CD4, CD8) for patients with low, moderate, and high microenvironment subtypes in this trial. Scale bar=100 μm.

FIG. 8b shows relative density of markers in an exemplary embodiment of the disclosure. Heatmap showing relative density (cells per mm²) of markers stratified by microenvironment subtype as determined by PAM clustering of immune cell type scores from gene expression deconvolution.

FIG. 8c shows differences in density of markers in an exemplary embodiment of the disclosure. Forest plot summarizing fold differences in density of markers for 6 patients who did not have objective response to treatment (shown in black) with expression data at both disease progression and baseline. Dot denotes average and bars represent 95% CI. Light grey dot denotes one additional patient with objective partial response to treatment.

FIG. 8d shows immunofluorescence images in an exemplary embodiment of the disclosure. Representative immunofluorescence images of samples with different microenvironment subtypes as determined by gene expression analysis. Scale bar=100 μm.

FIG. 8e shows immunofluorescence images in patients with response in an exemplary embodiment of the disclosure. Immunofluorescence images of patient with objective response before (left) and after (right) treatment. Scale bar=150 μm.

FIG. 9a shows anti-adenovirus antibody levels in an exemplary embodiment of the disclosure. Immunoglobulin G (IgG) levels before and after treatment across dose cohorts in the trial.

FIG. 9b shows anti-adenovirus antibody levels in an exemplary embodiment of the disclosure. IgG levels across dose cohorts in the trial over time (5×10⁸ vp, n=3; 5×10⁹ vp, n=3; 5×10¹⁰ vp, n=35).

FIG. 9c shows a survival curve in an exemplary embodiment of the disclosure. Kaplan-Meier survival curve with associated 95% CI. Patients stratified according to level of response in Anti-Ad5 IgG levels after treatment compared to baseline levels. +denote censored data. Shows survival using 4-fold threshold (HR 1.07, 95% CI 0.52 to 1.19, long-rank test p=0.86).

FIG. 9d shows a survival curve in an exemplary embodiment of the disclosure. Kaplan-Meier survival curve with associated 95% CI. Patients stratified according to level of response in Anti-Ad5 IgG levels after treatment compared to baseline levels. +denote censored data. Shows survival using 10-fold threshold (HR 1.41, 95% CI 0.35 to 1.4, long-rank test p=0.31).

FIG. 10 shows tumor size comparisons in an exemplary embodiment of the disclosure. Tumor size comparisons between responders and non-responders in patients who received declared dose DNX-2401 and pembrolizumab (n=42 independent samples). Shown are the distribution of maximal tumor size in mm for each patient. On the left are patients who had either stable disease (SD) or progressive disease (PD), and on the right are patients who had complete response (CR) or partial response (PR). Central bars of boxplots indicate medians, the box defines the upper and lower quartiles of the distribution, and the whiskers define the 1.5×interquartile range (IQR). Wilcoxon, p=0.38.

FIG. 11 shows survival of patients in an exemplary embodiment of the disclosure. Survival of patients treated with 5×10¹⁰ DNX-2401 and pembrolizumab. +denote censored data. Patients stratified according to response to tumor expression of PD-L1 by immunohistochemistry (n=18 positive and n=18 negative). HR 1.06 (95% CI 0.53 to 2.13)

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least±5% of the modified term if this deviation would not negate the meaning of the word it modifies.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

It should also be understood that, in certain methods described herein that include more than one step or act, the order of the steps or acts of the method is not necessarily limited to the order in which the steps or acts of the method are recited unless the context indicates otherwise.

The term “biopsy sample” as used herein refers to a sample obtained by biopsy, for example using a surgical approach such as an open approach or a stereotactic approach. A biopsy sample is, for example, a sample specimen from a tumor.

The term “nucleic acid” as used herein may refer to a biopolymer comprising monomers of nucleotides, such as deoxyribonucleic acid (DNA), ribonucleic acid (RNA) and other polynucleotides of modified nucleotides and/or nucleotide derivatives, and may be either double stranded (ds) or single stranded (ss). “Modified” bases include, for example, tritiated bases and unusual bases such as inosine. A variety of modifications can be made to DNA and RNA; thus “nucleic acid molecule”, “DNA molecule”, and “RNA molecule” embrace chemically, enzymatically, or metabolically modified forms. Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine or fluorophore and quencher conjugated nucleotides. Alternatively, the nucleic acid molecules can be produced biologically using an expression vector. In some embodiments, modified nucleotides comprise one or more modified bases (e.g. unusual bases such as inosine, and functional modifications to the bases such as amino modifications), modified backbones (e.g. peptide nucleic acid, PNA) and/or other chemically, enzymatically, or metabolically modified forms. The term “functional fragment” as used herein refers to a fragment of the nucleic acid that retains the functional property of the full-length nucleic acid. In some embodiments, modified nucleotides may contain one or more modified bases (e.g. unusual bases such as inosine, and functional modifications to the bases such as amino), modified backbones (e.g. peptide nucleic acid, PNA) and/or other chemically, enzymatically, or metabolically modified forms.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description. The recitation of numerical ranges by endpoints herein includes all numbers and fractions subsumed within that range (e.g. 1 to 5 includes for example 1, 1.5, 2, 2.75, 3, 3.90, 4, and 5). It is also to be understood that all numbers and fractions thereof are presumed to be modified by the term “about”.

It will be understood that any component defined herein as being included can be explicitly excluded by way of proviso or negative limitation, such as any specific compounds or method steps, whether implicitly or explicitly defined herein.

Unless otherwise indicated, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### II. Methods

The present inventors have shown that a safe dose of DNX-2401 combined with pembrolizumab results in objective and durable responses, including two complete responses, and survival benefit for select patients across multiple institutions. Further, the present inventors have identified that a particular tumor microenvironment is indicative of whether the tumor will respond to such combination therapy.

Accordingly, in one aspect, the present disclosure provides a method of selecting therapy for a subject with glioma comprising:
(a) determining a sample immune gene expression profile of a biopsy sample obtained from the subject comprising markers of immune infiltration by detecting the levels of mRNAs of cell type markers, immune checkpoint genes, functional orientation markers, and signature scores;
(b) comparing the values of (a) with a control profile comprising gene expression values from reference samples with known annotation of high (TMEhigh), medium (TMEmedium) and low (TMElow) tumor microenvironments;
(c) determining the level of similarity of (a) to (b), and
(d) selecting combination therapy of an oncolytic virus and an anti-PD-1 antibody or a binding fragment thereof if there is a high level of similarity of (a) to TMEmedium; a low level of similarity of (a) to TMEhigh or TMElow; and/or a higher level of similarity of (a) to TMEmedium than to TMEhigh or TMElow, and not selecting combination therapy of an oncolytic virus and an anti-PD-1 antibody or a binding fragment thereof if there is a low level of similarity of (a) to TMEmedium; a high level of similarity of (a) to TMEhigh or TMElow; and/or a higher level of similarity of (a) to TMEhigh or TMElow than to TMEmedium.

In an embodiment, the method further comprises a step of obtaining a biopsy sample from the subject prior to step (a).

In an embodiment, if no combination therapy is selected, the method further comprises testing the tumor microenvironment for other immune checkpoint gene expression levels. Optionally the method further comprises selecting combination therapy of an oncolytic virus and antibodies to the other immune checkpoint genes detected and anti-PD-1 antibody or a binding fragment thereof if PD-1 is detected.

In another embodiment, if combination therapy is selected and the subject suffers a recurrence, the method further comprises:
(e) determining a second sample immune gene expression profile of a second biopsy sample obtained from the subject comprising markers of immune infiltration by detecting the levels of mRNAs of cell type markers, immune checkpoint genes, functional orientation markers, and signature scores;
(f) comparing the values of (e) with a control profile comprising gene expression values from reference samples with known annotation of high (TMEhigh), medium (TMEmedium) and low (TMElow) tumor microenvironments; and
(g) determining the level of similarity of (e) to (f); and
(h) selecting combination therapy of an oncolytic virus and antibodies to immune checkpoint genes detected in the sample if there is a high level of similarity of (e) to TMEmedium; a low level of similarity of (e) to TMEhigh or TMElow; and/or a higher level of similarity of (e) to TMEmedium than to TMEhigh or TMElow, and not selecting combination therapy of an oncolytic virus and antibodies to immune checkpoint genes detected if there is a low level of similarity of (e) to TMEmedium; a high level of similarity of (e) to TMEhigh or TMElow; and/or a higher level of similarity of (e) to TMEhigh or TMElow than to TMEmedium.

In an embodiment, the method further comprises a step of obtaining a second biopsy sample from the subject prior to step (e).

In another aspect, herein provided is a method of treating a subject with glioma comprising:
(a) determining a sample immune gene expression profile of a biopsy sample obtained from the subject comprising markers of immune infiltration by detecting the levels of mRNAs of cell type markers, immune checkpoint genes, functional orientation markers, and signature scores;
(b) comparing the values of (a) with a control profile comprising gene expression values from reference samples with known annotation of high (TMEhigh), medium (TMEmedium) and low (TMElow) tumor microenvironments;
(c) determining the level of similarity of (a) to (b); and
(d) administering combination therapy of an oncolytic virus and an anti-PD-1 antibody or a binding fragment thereof to the subject if there is a high level of similarity of (a) to TMEmedium; a low level of similarity of (a) to TMEhigh or TMElow; and/or a higher level of similarity of (a) to TMEmedium than to TMEhigh or TMElow; and not administering combination therapy of an oncolytic virus and an anti-PD-1 antibody or a binding fragment thereof to the subject if there is a low level of similarity of (a) to TMEmedium; a high level of similarity to TMEhigh or TMElow; and/or a higher level of similarity of (a) to TMEhigh or TME low than to TMEmedium.

In an embodiment, the method further comprises a step of obtaining a biopsy sample from the subject prior to step (a).

In an embodiment, if no combination therapy is administered, the method further comprises testing the tumor microenvironment for other immune checkpoint gene expression levels. Optionally the method further comprises administering combination therapy comprising an oncolytic virus and antibodies to the other immune checkpoint genes detected and anti-PD-1 antibody or a binding fragment thereof if PD-1 is detected.

In another embodiment, if combination therapy is administered and the subject suffers a recurrence, the method further comprises:
(e) determining a second sample immune gene expression profile of a second biopsy sample obtained from the subject comprising markers of immune infiltration by detecting the levels of mRNAs of cell type markers, immune checkpoint genes, functional orientation markers, and signature scores;
(f) comparing the values of (e) with a control profile comprising gene expression values from reference samples with known annotation of high (TMEhigh), medium (TMEmedium) and low (TMElow) tumor microenvironments; and
(g) determining the level of similarity of (e) to (f); and
(h) administering combination therapy of an oncolytic virus and antibodies to immune checkpoint genes detected in the sample if there is a high level of similarity of (e) to TMEmedium; a low level of similarity of (e) to TMEhigh or TMElow; and/or a higher level of similarity of (e) to TMEmedium than to TMEhigh or TMElow, and not administering combination therapy of an oncolytic virus and antibodies to immune checkpoint genes detected if there is a low level of similarity of (e) to TMEmedium; a high level of similarity of (e) to TMEhigh or TMElow; and/or a higher level of similarity of (e) to TMEhigh or TMElow than to TMEmedium.

In an embodiment, the method further comprises a step of obtaining a second biopsy sample from the subject prior to step (e).

The term “glioma”, as used herein refers to a WHO grouping of three classes of diffuse gliomas, including astrocytoma, oligodendroglioma, and glioblastoma. The term “glioblastoma” as used herein refers to a WHO grade 4 CNS tumor. The term “gliosarcoma” as used herein refers to a subtype of glioblastoma.

The term “TMEhigh” as used herein refers to a tumor microenvironment (TME) enriched with immune cell infiltrates and that highly expresses multiple different suppressive immune checkpoints, leading to an exhaustive immune microenvironment by complementary mechanisms.

The term “TMEmedium” as used herein refers to a TME with a moderate degree of immune cells and that expresses moderate levels of PD-1 but low levels of additional immune checkpoint genes.

The term “TMElow” as used herein refers to a TME with low levels of immune cells and low expression of immune checkpoint genes.

In an embodiment, a higher level of similarity to the control profile is indicated by a higher correlation value computed between the sample immune gene expression profile and the control profile, optionally wherein the correlation value is a correlation coefficient. In another embodiment, a lower level of similarity to the control profile is indicated by a lower correlation value computed between the sample immune gene expression profile and control profile, optionally wherein the correlation value is a correlation coefficient. In one embodiment, the correlation coefficient is a linear coefficient, optionally a Pearson correlation coefficient or a Spearman correlation coefficient.

In another embodiment, a high level of similarity is indicated by a Pearson correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In yet another aspect, herein provided is a method of treating glioma comprising administering combined therapy of an oncolytic virus and anti-PD-1 antibody or a binding fragment thereof to a subject that has been previously identified as having a medium tumor microenvironment, wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

Also provided in an aspect is use of combined therapy of an oncolytic virus and anti-PD-1 antibody or a binding fragment thereof to treat glioma in a subject that has been previously identified as having a medium tumor microenvironment, wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

Further provided in an aspect is use of combined therapy of an oncolytic virus and anti-PD-1 antibody or a binding fragment thereof to treat glioma in a subject that has been previously identified as having a medium tumor microenvironment according to a method herein disclosed.

Also provided in an aspect is use of combined therapy of an oncolytic virus and anti-PD-1 antibody or a binding fragment thereof in a manufacture of a medicament for treating treat glioma in a subject that has been previously identified as having a medium tumor microenvironment, wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

Further provided in an aspect is of combined therapy of an oncolytic virus and anti-PD-1 antibody or a binding fragment thereof in a manufacture of a medicament for treating treat glioma in a subject that has been previously identified as having a medium tumor microenvironment according to a method herein disclosed.

Also provided in an aspect is combined therapy of an oncolytic virus and anti-PD-1 antibody or a binding fragment thereof for use in the treatment of treat glioma in a subject that has been previously identified as having a medium tumor microenvironment, wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

Further provided in an aspect is combined therapy of an oncolytic virus and anti-PD-1 antibody or a binding fragment thereof for use in the treatment of treat glioma in a subject that has been previously identified as having a medium tumor microenvironment according to a method herein disclosed.

In some embodiments, the cell type markers comprise B-cells, CD45, CD8 T cells, Cytotoxic cells, Dendritic cells, exhausted CD8 cells, macrophages, neutrophils, NK CD56dim cells, NK cells, T-cells, Gh1 cells and Treg cells.

In an embodiment, the B-cell markers comprise BLK, CD19, MS4A1 and TNFRSF17.

BLK can be from any organism or source, and optionally as shown in (NM_001715.3; or NM_001330465.2). CD19 can be from any organism or source, and optionally as shown in (NM_001178098.2, NM_001770.6, or NM_001385732.1). MS4A1 can be from any organism or source, and optionally as shown in (NG_023388.1, NM_152866.3, NM_152867.2, or NM_021950.4). TNFRSF17 can be from any organism or source, and optionally as shown in (NM_001192.3).

In an embodiment, the CD45 marker comprises PTPRC.

PTPRC can be from any organism or source, and optionally as shown in (NG_007730.2, NM_002838.5, NM_080921.4, or NM_001267798.2).

In an embodiment, the CD8 T cell markers comprise CD8A and CD8B.

CD8A can be from any organism or source, and optionally as shown in (NG_011608.2, NM_001768.7, NM_171827.4, NM_001145873.1, or NM_001382698.1). CD8B can be from any organism or source, and optionally as shown in (NM_172213.5, NM_172101.5, NM_172102.5, NM_004931.5, or NM_001178100.2).

In an embodiment, the cytotoxic cell markers comprise CTSW, GNLY, GZMA, GZMB, GZMH, KLRB1, KLRD1, KLRK1 and PRF1.

CTSW can be from any organism or source, and optionally as shown in (NM_001335.4). GNLY can be from any organism or source, and optionally as shown in (NM_001302758.2, NM_006433.5, or NM_012483.4). GZMA can be from any organism or source, and optionally as shown in (NM_006144.4). GZMB can be from any organism or source, and optionally as shown in (NM_004131.6, NM_001346011.2). KLRB1 can be from any organism or source, and optionally as shown in (NM_002258.3). KLRD1 can be from any organism or source, and optionally as shown in (NM_002262.5, NM_007334.3, NM_001114396.3, NM_001351060.2, NM 001351062.2, NM_001351063.2, NM_001414224.1, or NM_001414225.1). KLRK1 can be from any organism or source, and optionally as shown in (NG_027762.1, or NM_007360.4). PRF1 can be from any organism or source, and optionally as shown in (NM_005041.6, or NM_001083116.3).

In an embodiment, the dendritic cell markers comprise CCL13, CD209 and HSD11B1.

CCL13 can be from any organism or source, and optionally as shown in (NM_005408.3). CD209 can be from any organism or source, and optionally as shown in (NG_012167.1, NM_021155.4, NM_001144896.2, NM_001144897.2, NM_001144893.2, NM_001144894.2, NM_001144895.2, or NM_001144899.2). HSD11B1 can be from any organism or source, and optionally as shown in (NG_012081.1, NM_005525.4, NM_181755.3, or NM_001206741.2).

In an embodiment, the exhausted CD8 cell markers comprise CD244, EOMES and LAG3.

CD244 can be from any organism or source, and optionally as shown in (NM_016382.4, NM_001166663.2, or NM_001166664.2). EOMES can be from any organism or source, and optionally as shown in (NG_042182.1, NM_001278182.2, NM_005442.4, or NM_001278183.2). LAG3 can be from any organism or source, and optionally as shown in (NM_002286.6, NM_001414176.1, or NM_001414177.1).

In an embodiment, the macrophage markers comprise CD163, CD68 and CD84.

CD163 can be from any organism or source, and optionally as shown in (NG_029826.1, NM_004244.6, NM_203416.4, NM_001370145.1, or NM_001370146.1). CD68 can be from any organism or source, and optionally as shown in (NM_001251.3, or NM_001040059.2). CD84 can be from any organism or source, and optionally as shown in (NM_001184879.2, NM_003874.4, NM_001184881.2, NM_001184882.2, or NM 001330742.2).

In an embodiment, the mast cell markers comprise MS4A2 and TPSAB1/B2.

MS4A2 can be from any organism or source, and optionally as shown in (NM_000139.5, or NM_001256916.2). TPSAB1/B2 can be from any organism or source, and optionally as shown in (NM_003294.4).

In an embodiment, the neutrophil cell markers comprise CSF3R, FCGR3A/B and S100A12.

CSF3R can be from any organism or source, and optionally as shown in (NM_000760.4, NM_156039.3, or NM_172313.3). FCGR3A can be from any organism or source, and optionally as shown in (NM_000569.8, NM_001127592.2, NM_001127593.1, NM_001127595.2, NM_001127596.2, NM_001329120.2, or NM_001329122.1). FCGR3B can be from any organism or source, and optionally as shown in (NG_032926.2, NM_001244753.2, NM_000570.5, NM_001271035.2, NM_001271036.2, NM_001271037.2). S100A12 can be from any organism or source, and optionally as shown in (NG_032926.2, NM_001244753.2, NM_000570.5, NM_001271035.2, NM_001271036.2, or NM_001271037.2).

In an embodiment, the NKCD56 dim cell markers comprise IL21R, KIR2DL3, KIR3DL1 and KIR3DL2.

IL21R can be from any organism or source, and optionally as shown in (NG_012222.1, NM_021798.4, NM_181078.3, or NM_181079.5). KIR2DL3 can be from any organism or source, and optionally as shown in (NG_046935.1, or NM_015868.3). KIR3DL1 can be from any organism or source, and optionally as shown in (NG_021414.2, NM_177749.4, or NM_001310690.1). KIR3DL2 can be from any organism or source, and optionally as shown in (NM_006737.4, or NM_001242867.2).

In an embodiment, the NK cell marker comprises NCR1.

NCR1 can be from any organism or source, and optionally as shown in (KJ892768.1).

In an embodiment, the T-cell marker comprises CD3D, CD3E, CD3G, CD6 and SH2D1A.

CD3D can be from any organism or source, and optionally as shown in (NM_000732.6, or NM_001040651.2). CD3E can be from any organism or source, and optionally as shown in (NM_000733.4). CD3G can be from any organism or source, and optionally as shown in (NM_000073.3). CD6 can be from any organism or source, and optionally as shown in (NM_006725.5, NM_001254750.2, or NM_001254751.2). SH2DIA can be from any organism or source, and optionally as shown in (NM_002351.5, or NM_001114937.3).

In an embodiment, the Th1 cell marker comprises TBX21.

TBX21 can be from any organism or source, and optionally as shown in (NG_012166.1, or NM_013351.2).

In an embodiment, the Treg cell marker comprises FOXP3.

FOXP3 can be from any organism or source, and optionally as shown in (NM_014009.4, or NM_001114377.2).

In an embodiment, the immune checkpoint genes comprise PD-1 (PDCD1), PD-L1 (CD274), PD-L2 (PDCDILG2), CTLA4, TIM3, LAG3, TIGIT, B7-H3, IDO1, ICOS, NOS2, ARG2 and CXCL9.

PD-1, also known as PD1 or PDCDI, can be from any organism or source, and optionally as shown in (NG_012110.1, or NM_005018.3). PD-L1, also known as CD274, can be from any organism or source, and optionally as shown in (NM_014143.4, NM_001267706.2, or NM_001314029.2). PD-L2, also known as PDCDILG2, can be from any organism or source, and optionally as shown in (NM_025239.4). CTLA4 can be from any organism or source, and optionally as shown in (NG_011502.1, NM_005214.5, or NM_001037631.3). TIM3, also known as HAVCR2-hepatitis A virus cellular receptor 2, can be from any organism or source, and optionally as shown in (NG_030444.1, or NM_032782.5). LAG3 can be from any organism or source, and optionally as shown in (NM_002286.6, NM_001414176.1, or NM_001414177.1). TIGIT can be from any organism or source, and optionally as shown in (NM_173799.4). B7-H3, also known as CD276, can be from any organism or source, and optionally as shown in (NM_001024736.2, NM_025240.3, NM_001329628.2, or NM_001329629.2). IDO1 can be from any organism or source, and optionally as shown in (NM_002164.6). ICOS can be from any organism or source, and optionally as shown in (NM_012092.4). NOS2 can be from any organism or source, and optionally as shown in (NG_011470.1, NM_000625.4). ARG2 can be from any organism or source, and optionally as shown in (NG_011964.1, or NM_001172.4). CXCL9 can be from any organism or source, and optionally as shown in (NM_002416.3).

In some embodiments, the functional orientation markers comprise T cell activation, T cell inhibition, Class 1 MHC, regulatory T cells, Myeloid cell chemotaxis, MI markers, M2 markers, Tertiary lymphoid structures, angiogenic markers.

In an embodiment, the T cell activation markers comprise CD70, CD244, CD48 and CD44.

CD70 can be from any organism or source, and optionally as shown in (NM_001252.5, or NM_001330332.2). CD244 can be from any organism or source, and optionally as shown in (NG_015991.1, NM_016382.4, NM_001166663.2, or NM_001166664.2). CD48 can be from any organism or source, and optionally as shown in (NM_001778.4, or NM_001256030.2). CD44 can be from any organism or source, and optionally as shown in (NG_008937.1,NM_000610.4, NM_001001389.2, NM_001001390.2, NM_001001391.2, NM_001001392.2, NM_001202555.2, NM_001202556.2, or NM_001202557.2).

In an embodiment, the T cell inhibition markers comprise LAG3, TNFRSF8, CTLA4, TIGIT, PDCD1, HAVCR2, BTLA, ADORA2A, TNFRSF25 and LAIR1.

LAG3 can be from any organism or source, and optionally as shown in (NM_002286.6, NM_001414176.1, or NM_001414177.1). TNFRSF8 can be from any organism or source, and optionally as shown in (NM_001243.5, or NM_001281430.3). CTLA4 can be from any organism or source, and optionally as shown in (see above). TIGIT can be from any organism or source, and optionally as shown in (see above). PDCDI can be from any organism or source, and optionally as shown in (see above). HAVCR2 can be from any organism or source, and optionally as shown in (see above). BTLA can be from any organism or source, and optionally as shown in (NM_181780.4, or NM_001085357.2). ADORA2A can be from any organism or source, and optionally as shown in (NG_052804.1, NM_001278497.2, NM_001278498.2,NM_000675.6, NM_001278499.2, or NM_001278500.2). TNFRSF25 can be from any organism or source, and optionally as shown in (NG_029910.1, NM_148965.2, NM_003790.3, NM_148966.2, NM_148967.2, NM_148970.2, or NM_001039664.2). LAIR1 can be from any organism or source, and optionally as shown in (NM_002287.6, NM_001289026.3, or NM_001289027.3).

In an embodiment, the regulatory T cell markers comprise FOXP3 and TNFRSF18.

FOXP3 can be from any organism or source, and optionally as shown in (see above). TNFRSF18 can be from any organism or source, and optionally as shown in (NM_004195.3, NM_148901.2, or NM_148902.2).

In an embodiment, the Class 1 MHC markers comprise HLA-A, HLA-B, HLA-C, HLA-E, HLA-F and B2M.

HLA-A can be from any organism or source, and optionally as shown in (Z46633.1, or D38525.1). HLA-B can be from any organism or source, and optionally as shown in (D83043.1). HLA-C can be from any organism or source, and optionally as shown in (NG_029422.3, NM_002117.6, or NM_001243042.1). HLA-E can be from any organism or source, and optionally as shown in (NM_005516.6). HLA-F can be from any organism or source, and optionally as shown in (NG_012009.2, NM_001098479.2, NM_018950.3, or NM_001098478.2). B2M can be from any organism or source, and optionally as shown in (NM_213978.1).

In an embodiment, the myeloid cell chemotaxis markers comprise CCL2, CCL5, VEGFA and CSF1.

CCL2 can be from any organism or source, and optionally as shown in (NM_002982.4). CCL5 can be from any organism or source, and optionally as shown in (NM_002985.3, or NM_001278736.2). VEGFA can be from any organism or source, and optionally as shown in (NM_001171623.2, NM_001171624.2, NM_001171625.2, NM_001171626.2, NM_001171627.2, NM_001171628.2, NM_001171629.2, or NM_001171630.2). CSF1 can be from any organism or source, and optionally as shown in (NM_000757.6, NM_172210.3, NM_172211.4, or NM_172212.3).

In an embodiment, the MI markers comprise CCL2, CXCL10, GBP2, IFIT3, SLAMF7, CXCL9, CCL8, IL1B and CD38.

CCL2 can be from any organism or source, and optionally as shown in (see above). CXCL10 can be from any organism or source, and optionally as shown in (NM_001565.4). GBP2 can be from any organism or source, and optionally as shown in (NM_004120.5). IFIT3 can be from any organism or source, and optionally as shown in (NM_001549.6, NM_001031683.4, NM_001289758.2, or NM_001289759.2). SLAMF7 can be from any organism or source, and optionally as shown in (NM_021181.5, NM_001282588.2, NM_001282589.2, NM_001282590.2, NM_001282591.2, NM 001282592.2, NM_001282593.2, NM_001282595.1, NM_001282594.2, or NM_001282596.2). CXCL9 can be from any organism or source, and optionally as shown in (NM_002416.3). CCL8 can be from any organism or source, and optionally as shown in (NM_005623.3). IL1B can be from any organism or source, and optionally as shown in (NM_000576.3). CD38 can be from any organism or source, and optionally as shown in (NM_001775.4).

In an embodiment, the M2 markers comprise CXCL16, CXCR4, CD14, MRC1, ARGI and CCL13.

CXCL16 can be from any organism or source, and optionally as shown in (NM_001386809.1, or NM_001100812.2). CXCR4 can be from any organism or source, and optionally as shown in (NM_001008540.2, NM_003467.3, NM_001348056.2, NM_001348059.2, or NM_001348060.2). CD14 can be from any organism or source, and optionally as shown in (NM_000591.4, NM_001040021.3, NM_001174104.2, or NM_001174105.2). MRC1 can be from any organism or source, and optionally as shown in (NM_002438.4). ARGI can be from any organism or source, and optionally as shown in (NM_001244438.2, NM_000045.4, or NM_001369020.1). CCL13 can be from any organism or source, and optionally as shown in (NM_005408.3).

In an embodiment, the tertiary lymphoid structure markers comprise CCL2, CCL4, CCL5, CCL8, CCL18, CCL19, CCL21, CXCL9, CXCL10, CXCL11 and CXCL13.

CCL2 can be from any organism or source, and optionally as shown in (see above). CCL4 can be from any organism or source, and optionally as shown in (NM_002984.4). CCL5 can be from any organism or source, and optionally as shown in (NM_002985.3, or NM_001278736.2). CCL8 can be from any organism or source, and optionally as shown in (NM_005623.3). CCL18 can be from any organism or source, and optionally as shown in (NM_002988.4). CCL19 can be from any organism or source, and optionally as shown in (NM_006274.3). CCL21 can be from any organism or source, and optionally as shown in (NM_002989.4). CXCL9 can be from any organism or source, and optionally as shown in (see above). CXCL10 can be from any organism or source, and optionally as shown in (see above). CXCL11 can be from any organism or source, and optionally as shown in (NM_005409.5, or NM_001302123.2). CXCL13 can be from any organism or source, and optionally as shown in (NM_006419.3, or NM_001371558.1).

In an embodiment, the angiogenic markers comprise VEGFA, VEGFB, KDR, CXCR2, HIFIA and ANGPT2.

VEGFA can be from any organism or source, and optionally as shown in (see above). VEGFB can be from any organism or source, and optionally as shown in (NG_029823.1, NM_001243733.2, or NM_003377.5). KDR can be from any organism or source, and optionally as shown in (NM_002253.4). CXCR2 can be from any organism or source, and optionally as shown in (NM_001557.4, NM_001168298.2). HIFIA can be from any organism or source, and optionally as shown in (NG_029606.1). ANGPT2 can be from any organism or source, and optionally as shown in (NM_001147.3, NM_001118887.2, NM_001118888.2, NM_001386335.1, NM_001386336.1, or NM_001386337.1).

In some embodiments, the signature scores comprise chemokine, cytolytic, interferon gamma signaling, interferon gamma downstream signals and T cell inflamed.

In an embodiment, the chemokine markers comprise CCL2, CCL4, CCL5, CCL8, CCL18, CCL19, CCL21, CXCL9, CXCL10, CXCL11 and CXCL13.

In an embodiment, the cytolytic markers comprise GZMA and PRF1.

GZMA can be from any organism or source, and optionally as shown in (see above). PRF1 can be from any organism or source, and optionally as shown in (see above).

In an embodiment, the interferon signaling markers comprise IDO1, CXCL10, CXCL9, HLA-DRA, STAT1 and IFNG.

IDO1 can be from any organism or source, and optionally as shown in (see above). CXCL10 can be from any organism or source, and optionally as shown in (see above). CXCL9 can be from any organism or source, and optionally as shown in (see above). HLA-DRA can be from any organism or source, and optionally as shown in (see above). STAT1 can be from any organism or source, and optionally as shown in (NM_001384880.1, NM_001384881.1, NM_001384882.1, NM_001384883.1, NM_001384885.1, NM_001384886.1, NM_001384887.1, NM_001384889.1, or NM_001384890.1). IFNG can be from any organism or source, and optionally as shown in (NM_000619.3).

In an embodiment, the interferon gamma downstream signal markers comprise CD3D, IDO1, CD3E, CCL5, GZMK, CD2, HLA-DRA, CXCL13, IL2RG, NKG7, HLA-E, CXCR6, LAG3, CXCL10, STAT1 and GZMB.

CD3D can be from any organism or source, and optionally as shown in (see above). IDO1 can be from any organism or source, and optionally as shown in (see above). CD3E can be from any organism or source, and optionally as shown in (see above). CCL5 can be from any organism or source, and optionally as shown in (NM_002985.3, or NM_001278736.2). GZMK can be from any organism or source, and optionally as shown in (NM_002104.3). CD2 can be from any organism or source, and optionally as shown in (NM_001328609.2, or NM_001767.5). HLA-DRA can be from any organism or source, and optionally as shown in (see above). CXCL13 can be from any organism or source, and optionally as shown in (see above). IL2RG can be from any organism or source, and optionally as shown in (NM_000206.3). NKG7 can be from any organism or source, and optionally as shown in (NM_005601.4, or NM_001363693.2). HLA-E can be from any organism or source, and optionally as shown in (NM_005516.6). CXCR6 can be from any organism or source, and optionally as shown in (see above). LAG3 can be from any organism or source, and optionally as shown in (NM_002286.6, NM_001414176.1, or NM_001414177.1). CXCL10 can be from any organism or source, and optionally as shown in (see above). STAT1 can be from any organism or source, and optionally as shown in (see above). GZMB can be from any organism or source, and optionally as shown in (see above).

In an embodiment, the T cell inflamed markers comprise CCL5, CD27, CD274 [PD-L1], CD276 [B7-H3], CD8A, CMKLR1, CXCL9, CXCR6, HLA.DQA1, HLA.DRB1, HLA.E, IDO1, LAG3, NKG7, PDCDILG2 [PD-L2], PSMB10, STAT1, and TIGIT.

CCL5 can be from any organism or source, and optionally as shown in (see above). CD27 can be from any organism or source, and optionally as shown in (NM_001413263.1, NM_001242.5, NM_001413264.1, NM_001413265.1, NM_001413266.1, NM_001413267.1, or NM_001413268.1). CD274, also known as PD-L1, can be from any organism or source, and optionally as shown in (see above). CD276, also known as B7-H3 can be from any organism or source, and optionally as shown in (see above). CD8A can be from any organism or source, and optionally as shown in (see above). CMKLR1 can be from any organism or source, and optionally as shown in (NM_001142343.2, NM_004072.3, NM_001142344.2, or NM_001142345.2). CXCL9 can be from any organism or source, and optionally as shown in (see above). CXCR6 can be from any organism or source, and optionally as shown in (see above). HLA.DQA1 can be from any organism or source, and optionally as shown in (NG_032876.1, or NM_002122.5). HLA-DRB1 can be from any organism or source, and optionally as shown in (NM_002124.4, NM_001243965.1, NM_001359193.1, or NM_001359194.1). HLA.E can be from any organism or source, and optionally as shown in (NM_005516.6). IDO1 can be from any organism or source, and optionally as shown in (see above). LAG3 can be from any organism or source, and optionally as shown in (NM_002286.6, NM_001414176.1, or NM_001414177.1). NKG7 can be from any organism or source, and optionally as shown in (NM_005601.4, or NM_001363693.2). PDCDILG2, also known as PD-L2, can be from any organism or source, and optionally as shown in (see above). PSMB10 can be from any organism or source, and optionally as shown in (NM_002801.4). STAT1 can be from any organism or source, and optionally as shown in (see above). TIGIT can be from any organism or source, and optionally as shown in (see above).

In some embodiments, testing the sample for cell type markers, immune checkpoint genes, functional orientation markers, and signature scores comprises or consists of measuring gene expression level of ADORA2A, ANGPT2, ARG1, B2M, BLK, BTLA, CCL13, CCL18, CCL19, CCL2, CCL21, CCL4, CCL5, CCL8, CD14, CD163, CD19, CD2, CD209, CD244, CD38, CD3D, CD3E, CD3G, CD44, CD48, CD6, CD68, CD70, CD84, CD8A, CD8B, CSF1, CSF3R, CTLA4, CTSW, CXCL10, CXCL11, CXCL13, CXCL16, CXCL9, CXCR2, CXCR4, CXCR6, EOMES, FCGR3A/B, FOXP3, GBP2, GNLY, GZMA, GZMB, GZMH, GZMK, HAVCR2, HIFIA, HLA-A, HLA-B, HLA-C, HLA-DRA, HLA-E, HLA-F, HSD11B1, IDO1, IFIT3, IFNG, IL1B, IL21R, IL2RG, KDR, KIR2DL3, KIR3DL1, KIR3DL2, KLRB1, KLRD1, KLRK1, LAG3, LAIR1, MRC1, MS4A1, MS4A2, NCRI, NKG7, PDCD1, PRF1, PTPRC, S100A12, SH2DIA, SLAMF7, STAT1, TBX21, TIGIT, TNFRSF17, TNFRSF18, TNFRSF25, TNFRSF8, TPSAB1/B2, VEGFA, VEGFB.

In some embodiments, the glioma is glioblastoma.

The combination therapy of oncolytic virus and PD-1 blockade combines the initial local effects of the oncolytic virus on the tumor microenvironment with the systemic effects of innate and adaptive immune responses from virus replication and PD-1 inhibition. In an embodiment, the oncolytic virus is administered sequentially with the anti-PD-1 antibody or the binding fragment thereof. For example, a single dose of oncolytic virus is administered, optionally by injection, at the time of tumor biopsy followed by administration of repeated doses of anti-PD-1 antibody or a binding fragment thereof.

Any oncolytic virus that is capable of reconditioning the tumor microenvironment towards a ‘hot’ phenotype is contemplated. In some embodiments, the oncolytic virus is an oncolytic adenovirus. In some embodiments, the oncolytic adenovirus is a conditionally replicative oncolytic adenovirus. In one embodiment, the oncolytic adenovirus is DNX-2401 (tasadenoturev; Delta-24-RGD), which is a conditionally replicative oncolytic adenovirus engineered to treat high-grade malignant gliomas, which contains two stable genetic changes in the adenovirus dsDNA genome that cause it to selectively and efficiently replicate in cancerous cells.

In an embodiment, 5×10⁸ to 5×10¹⁰ virus particles are administered intratumorally.

Administration will depend on the pharmacokinetics of the antibody and the oncolytic virus in the presence of each other and can include administering the oncolytic virus about a week prior to administration of the antibody. In some embodiments, the oncolytic virus is administered intratumorally prior to repeated doses of anti-PD-1 antibody or a binding fragment thereof.

In some embodiments, the repeated doses of anti-PD-1 antibody or a binding fragment thereof start about 7 days after administration of the oncolytic virus. In some embodiments, the anti-PD-1 antibody or the binding fragment thereof is administered or used by intravenous infusion over about 30 minutes. In an embodiment, about 200 mg of the anti-PD-1 antibody or the binding fragment thereof is infused intravenously. In an embodiment, the doses of anti-PD-1 antibody or the binding fragment thereof are administered as about 200 mg infused intravenously over about 30 days.

In an embodiment, administration or use of anti-PD-1 antibody or a binding fragment thereof is repeated every 3 weeks starting at 7 days after administration or use of the oncolytic virus. In an embodiment, the administration or use of the anti-PD-1 antibody or the binding fragment thereof is continued for at least 6 months. In an embodiment, the administration or use of the anti-PD-1 antibody or the binding fragment thereof is continued for at least 1 year. In another embodiment, the administration or use of the anti-PD-1 antibody or the binding fragment thereof is continued for at least 2 years. In another embodiment, the administration or use of the anti-PD-1 antibody or the binding fragment thereof is continued for at least 3 years. In another embodiment, the administration or use of the anti-PD-1 antibody or the binding fragment thereof is continued for at least 4 years. In another embodiment, the administration or use of the anti-PD-1 antibody or the binding fragment thereof is continued for at least 5 years. In another embodiment, the administration or use of the anti-PD-1 antibody or the binding fragment thereof is continued for at least 10 years. In another embodiment, the administration or use of the anti-PD-1 antibody or the binding fragment thereof is continued indefinitely.

The term “anti-PD-1 antibody” as used herein refers to an antibody that binds specifically to programmed cell death protein 1 (PD-1) optionally as shown in (MW051356.1, UMM61401, or UMM61400). In an embodiment the anti-PD-1 antibody is a monoclonal antibody.

The term “antibody” as used herein refers to an immunoglobulin molecule capable of specific binding to a target through at least one antigen recognition site, located in the variable region of the immunoglobulin molecule. The antibody may be from recombinant sources and/or produced in transgenic animals, and includes, without limitation, monoclonal antibodies, chimeric and humanized antibodies, and binding fragments thereof, including for example a single chain Fab fragment, Fab′2 fragment, or single chain Fv fragment. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2. Humanized or other chimeric antibodies may include sequences from one or more than one isotype, class, or species.

The basic antibody structural unit is known in the art to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. Within each of the VH and VL domains are three hypervariable regions or complementarity determining regions (CDRs, commonly denoted CDR-H1, CDR-H2, CDR-H3, CDR-L1, CDR-L2, and CDR-L3). The carboxy-terminal portions of the heavy and light chains together form a constant region primarily responsible for effector function. Further, these antibodies are typically produced as antigen binding fragments such as Fab, Fab′ F(ab′)2, Fd, Fv and single domain antibody fragments, or as single chain antibodies (e.g. scFv) in which the heavy and light chains are linked by a spacer or linker. The antibodies may include sequences from any suitable species including human. Also, the antibodies may exist in monomeric or polymeric form.

The term “antibody fragment” or “binding fragment” as used herein is intended to include without limitations Fab, Fab′, F(ab′)2, scFab, scFv, dsFv, ds-scFv, dimers, minibodies, diabodies, and multimers thereof, and Domain Antibodies. Antibodies can be fragmented using conventional techniques. For example, F(ab′) 2 fragments can be generated by treating the antibody with pepsin. The resulting F(ab′) 2 fragment can be treated to reduce disulfide bridges to produce Fab′ fragments. Papain digestion can lead to the formation of Fab fragments. Fab, Fab′ and F(ab′) 2, scFv, dsFv, ds-scFv, dimers, minibodies, diabodies, and other fragments can also be synthesized by recombinant techniques.

In some embodiments, the anti-PD-1 antibody comprises a light chain variable region and a heavy chain variable region, the heavy chain variable region comprising complementarity determining regions CDR-H1, CDR-H2 and CDR-H3, and the light chain variable region comprising complementarity determining region CDR-L1, CDR-L2 and CDR-L3, wherein the amino acid sequences of said CDRs comprise the sequences:

CDR-H1:
SEQ ID NO: 1
NYYMY;
CDR-H2:
SEQ ID NO: 2
GINPSNGGTNFNEKFK;
CDR-H3:
SEQ ID NO: 3
RDYRFDMGFDY;
CDR-L1:
SEQ ID NO: 4
RASKGVSTSGYSYLH
CDR-L2:
SEQ ID NO: 5
LASYLES;
and
CDR-L3:
SEQ ID NO: 6
QHSRDLPLT.

In some embodiments, the anti-PD-1 antibody or the binding fragment thereof is pembrolizumab or a binding fragment thereof.

The term “administering” or “administration” as used herein refers to the placement of an agent, a drug, a compound, a pharmaceutical composition, an inhibitor or a vaccine as disclosed herein into a subject by a method or route which results in at least partial delivery to a desired site. The compounds and compositions disclosed herein can be administered by any appropriate route which results in an effective treatment in the subject. Possible routes of administration of the compounds and pharmaceutical compositions disclosed herein include, but are not limited to, intravenous, intraperitoneal, intramuscular, subcutaneous, transdermal, oral, buccal, sublingual, intranasal, or rectal routes of administration, or a combination thereof.

The term “treating”, “treatment”, and the like, as used herein, and as is well understood in the art, refers to an approach for obtaining beneficial or desired results, including clinical results. Beneficial or desired clinical results include, but are not limited to alleviation or amelioration of one or more symptoms or conditions, arresting development of disease, diminishment of extent of disease, stabilized (i.e. not worsening) state of disease, preventing spread of disease, delay or slowing of disease progression, amelioration or palliation of the disease state, including regression of the disease, diminishment of the reoccurrence of disease, and remission (whether partial or total), whether detectable or undetectable. “Treating” and “treatment” may also refer to prolonging survival as compared to expected survival if not receiving treatment. “Treating” and “treatment” as used herein also include prophylactic treatment. The effect may be prophylactic in terms of completely or partially preventing a disease or symptom thereof and/or may be therapeutic in terms of affecting a partial or complete cure for a disease and/or symptoms of the disease. For example, a subject with early cancer can be treated to prevent progression, or alternatively a subject in remission can be treated to prevent recurrence. Prophylactic treatment includes preventing the disease or a symptom of a disease from occurring in a subject which may be predisposed to the disease but has not yet been diagnosed as having it (e.g., including diseases that may be associated with or caused by a primary disease).

Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the methods of the present disclosure to prevent, delay, alleviate, arrest or inhibit development of the symptoms or conditions associated with glioma.

The term “subject” as used herein refers to a human.

mRNA levels of each gene can be obtained by measuring mRNA expression, for example, by qPCR, or by directly quantifying mRNA, for example, by RNA-Sequencing or Nanostring. In an embodiment, mRNA levels are determined by measuring mRNA count.

The term “sample immune gene expression profile” or “sample gene expression profile” or “sample profile” as used herein refers to the levels of mRNA of the identified genes in the biopsy sample.

The control profile may be a reference value and/or may be derived from one or more samples, optionally from historical immune gene expression data from a pool of samples with known annotation of TMEmedium, TMEhigh or TMElow. In an embodiment, the control profile is a value that is continually updated as further samples are collected and immune gene expression levels are measured and correlated. It will be understood that the control profile represents an average of the levels for the selected genes described herein. Average values may, for example, be the mean values or median values.

For example, a “TMEmedium control profile” may be generated by measuring the mRNA levels of the specified genes for those samples with known annotation of TMEmedium.

Methods of determining the similarity between profiles are well known in the art. Methods of determining similarity may in some embodiments provide a non-quantitative measure of similarity, for example, using visual clustering. In other embodiments, similarity may be determined using methods which provide a quantitative measure of similarity.

In an embodiment, similarity may be measured by partition-around-medoid clustering. Partitioning around medoids is a method to find a series of objects (termed medoids) that are centrally located in clusters. This algorithm minimizes the average dissimilarity between objects.

In an embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r \text{?}$$

$$\text{?} \text{indicates text missing or illegible when filed}$$

In one embodiment, x and y are the expression values of the mRNA in a sample profile and a control profile, respectively.

In an embodiment, a correlation coefficient calculated between a sample immune gene expression profile and a control profile indicates a high level of similarity to the control profile when the correlation coefficient has an absolute value between 0.5 to 1, optionally between 0.75 to 1, and a low level of similarity to the control profile when the correlation coefficient has an absolute value between 0 to 0.5, optionally between 0 to 0.25.

It will be appreciated that any “correlation value” which provides a quantitative scaling measure of similarity between profiles may be used to measure similarity.

A sample immune gene expression profile may be identified as being indicative of a medium tumor environment (TMEmedium), where the sample profile has high similarity to the TMEmedium control profile, low similarity to the TMEhigh or TMElow specific control profile, or higher similarity to the TMEmedium specific control profile than to the TMEhigh or TMElow specific control profile. A sample profile may be identified as TMEhigh, where the sample immune gene expression profile has high similarity to the TMEhigh specific control profile, low similarity to the TMEmedium or TMElow specific control profile, or higher similarity to the TMEhigh specific control profile than to the TMEmedium or TMElow specific control profile. A sample profile may be identified as TMElow, where the sample immune gene expression profile has high similarity to the TMElow specific control profile, low similarity to the TMEmedium or TMEhigh specific control profile, or higher similarity to the TMElow specific control profile than to the TMEmedium or TMEhigh specific control profile.

For example, in an embodiment, a sample profile may be identified as indicative of a known annotation control based on calculation of a score, which generally is defined by the following formula:

score(B)=r(B, sample profile)−r(B, control profile)

where r is the Pearson correlation coefficient, and B is a vector of mRNA levels across the selected genes.

For example, a sample profile with a positive score is more similar to the TMEmedium specific control profile across the selected genes, and is therefore classified as indicative of a medium tumor microenvironment, whereas a sample with a negative score is more similar to the TMEhigh or TMElow specific control profile across the selected geens, and is classified as “not indicative of TMEmedium”.

In an embodiment, the gene expression profiles are analyzed using combinatorial or multivariate dimension reduction statistical or mathematical methods. Combinatorial or multivariate dimension reduction statistical or mathematical methods may be clustering analysis or desirability analyses to enable comparisons between gene profiles with dimension-reduced empirical values.

In an embodiment, the clustering analysis is principal component analysis. Principal component analysis is applied as unbiased tool to visualize similarity between the gene profiles of different groups (e.g. a TMEmedium specific control profile, a TMEhigh specific control profile, a TMElow specific control profile and a sample profile). In another embodiment, the clustering analysis is canonical correlation analysis. Canonical correlation analysis is applied as a supervised technique to test whether multivariate gene profiles of different groups are statistically distinct, and to provide information on their level of similarity.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### Examples

The following non-limiting examples are illustrative of the present disclosure:

### Example 1

### Results

### Patient Demographics and Baseline Characteristics

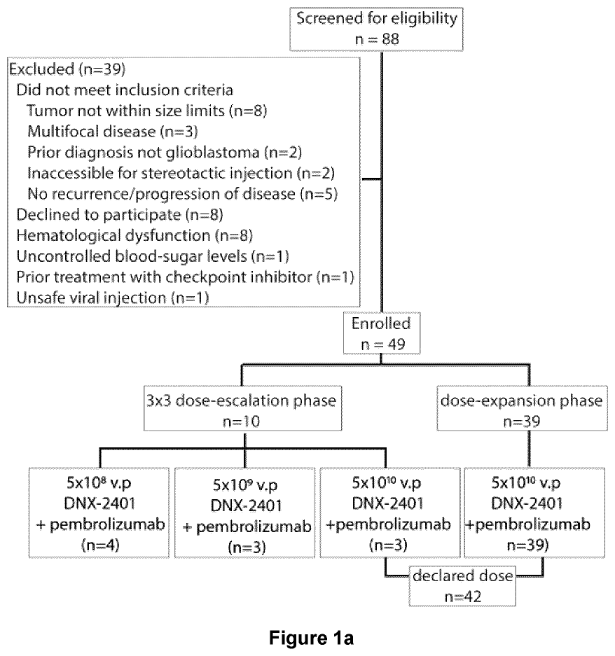

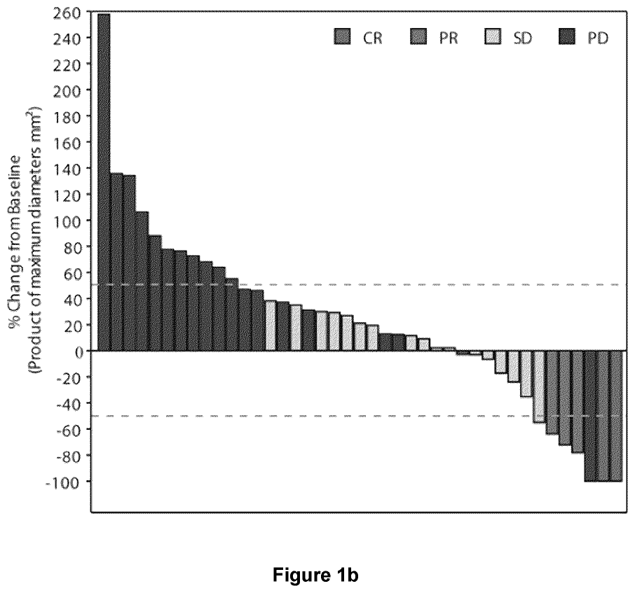

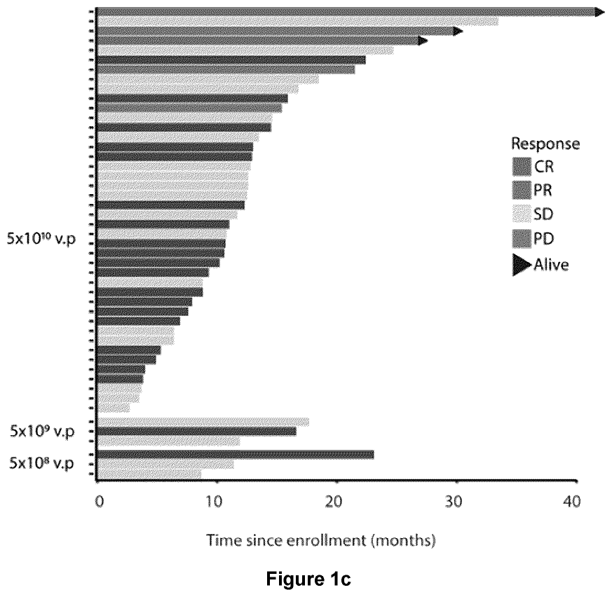

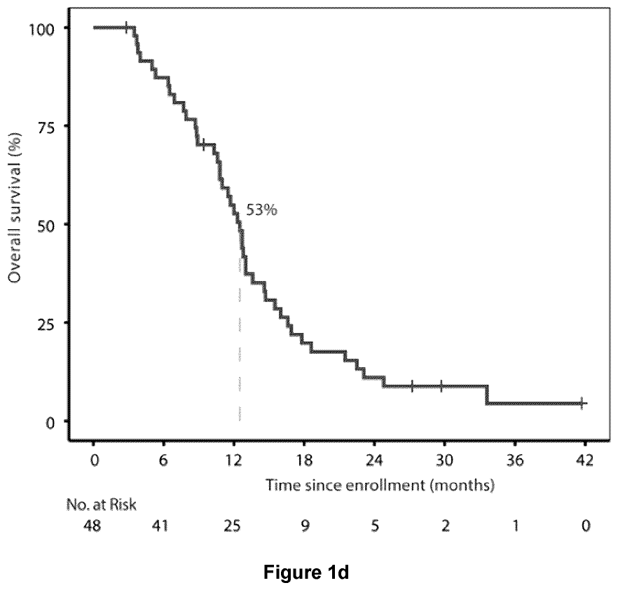

A total of 49 patients from 13 of the 15 participating institutions were enrolled between Sep. 28 2016 to Jan. 17 2019 (FIG. 1a). The demographic and baseline clinical characteristics of all patients enrolled are reported in Table 1. The median age of patients was 53 years and 41% were women. The majority of patients (80%) presented after first recurrence and 18% of patients were using steroids at baseline. All patients had histopathological diagnosis of glioblastomas, except one patient enrolled with gliosarcoma (2%). Most patients (90%, N=44) had reported IDH1 wildtype tumors, 4 (8%) had IDH1 mutant tumors, and IDH1 mutation status was not known for 1 patient. All patients had received prior treatment with temozolomide and radiotherapy, 6 (12%) patients had prior bevacizumab treatment, and 5 (10%) had prior treatment with a tumor-treating fields device.

### Safety

Forty-eight of 49 (98%) patients were treated with one dose of DNX-2401 after a standard biopsy, which was then followed by pembrolizumab starting 7 days later. One patient enrolled in the first dose cohort received 5×10⁸ vp DNX-2401 but did not start pembrolizumab due to delirium which was attributed by the investigators to anesthesia used during biopsy, unrelated to treatment. This patient was included in the safety analysis set only, per protocol. There were no dose-limiting toxicities observed and the maximal dose tested (5×10¹⁰ vp DNX-2401) was selected as the declared dose for the dose-expansion phase. In total, across both dose escalation and dose expansion phases, which were used to determine the safety and efficacy of DNX-2401 doses, patients were treated with 5×10⁸ (n=4), 5×10⁹ (n=3) and 5×10¹⁰ vp DNX-2401 (n=42). The median duration of exposure to treatment with DNX-2401 and pembrolizumab was 153 days (range 21 to 753 days), including three patients (6%) who completed the full 2-year course of pembrolizumab therapy.

An overview of adverse events in the study are summarized in Tables 5 to 6 and Table 9. Overall, DNX-2401 in combination with pembrolizumab was generally well tolerated and AEs were primarily as expected for patients with recurrent glioblastoma with the majority of these being grade 3 or lower events. There were no adverse events related to adenoviral infection. There were no deaths related to adverse events that were related to treatment. One patient died approximately 7 months after initiating treatment due to hyperosmolar hyperglycemic nonketotic acidosis, which was considered unrelated to treatment.

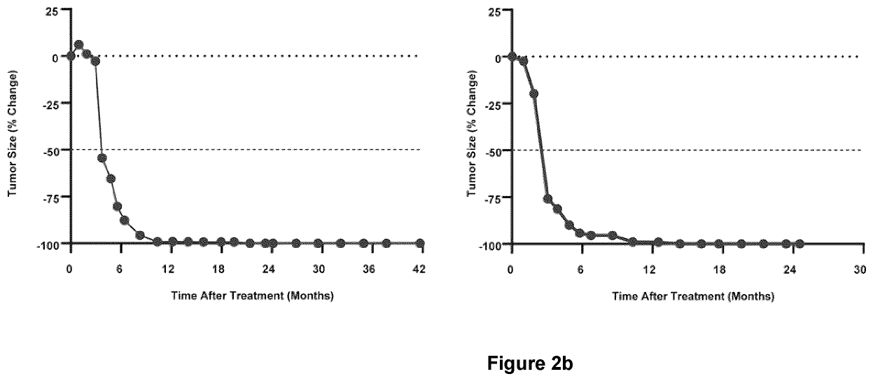

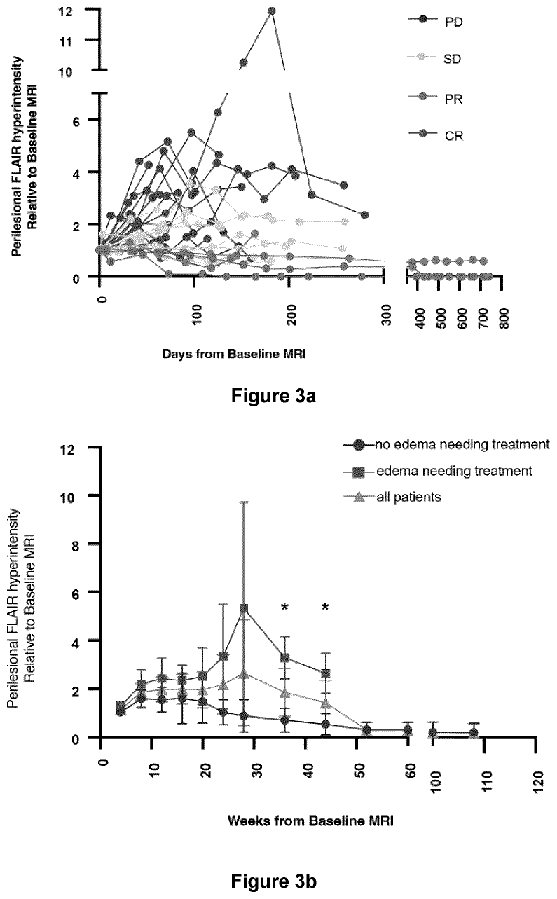

Adverse events that were considered to be related to treatment are summarized in Table 2. The majority of these events were grade 1 or 2 events, with the most common being brain edema (37%), headache (31%) and fatigue (29%). Longitudinal volumetric changes of perilesional edema are shown in FIG. 3a and FIG. 3b. Patients with and without symptomatic edema both had increases in volumetric measurements of perilesional edema from 8 weeks to 20 weeks after treatment. Patients who did not develop symptomatic edema begin to have a decrease in volume of perilesional edema after 20 weeks, whereas those who develop symptomatic edema continue to have increases in volume of perilesional edema after 20 weeks. Treatment-related serious adverse events that were noted in more than one patient included brain edema (16%), dysphasia (6%), and hemiparesis (6%). Serious cerebral edema was managed with either short-course dexamethasone (89%) and/or other concomitant supportive medications including bevacizumab (18%; Table 10). Surgical intervention was not needed for serious cerebral edema in any patient. Pembrolizumab was interrupted or discontinued for 4 patients who had cerebral edema but resumed after resolution. One patient had grade 3 cerebral edema, somnolence, and hemiparesis that started 23 days after initiation of treatment, leading to treatment discontinuation and resolution of the adverse event. A summary of serious adverse events related to treatment are provided in Table 11.

### Efficacy

The efficacy and survival endpoints are summarized in Table 3. According to mRANO criteria, 2 patients had a complete response, and 3 patients had a partial response (FIG. 1b and FIG. 1c) yielding an objective response rate of 10.4% (90% CI 4.2-20.7) in the intent to treat population and 11.9% (90% CI 4.8 to 23.4) for patients treated with the declared dose of DNX-2401, which was numerically greater than prespecified historical rate of 5% but did not meet statistical endpoint. One additional patient of interest had a complete response at the lesion where DNX-2401 was delivered approximately 8 months after treatment, however, a new lesion at a distant site was evident at the same assessment and the patient was therefore classified to have progressive disease. The median time to response was 3.0 months (range 1.9 to 17.4 months) and median duration of response was 9.4 months (range 1.8 to 33.7 months) in patients who showed an objective response. An additional 22 patients in the intent to treat population and 18 patients in the declared dose population had stable disease lasting longer than 28 days, which resulted in a clinical benefit rate of 56.2% (95% CI 41.1-70.5) and 54.8% (95% CI 38.7 to 70.2), respectively. The median duration of clinical benefit was 3.7 months (range 1.7 to 37.7 months). A summary of therapies received after treatment and at or after disease progression are summarized in Table 7.

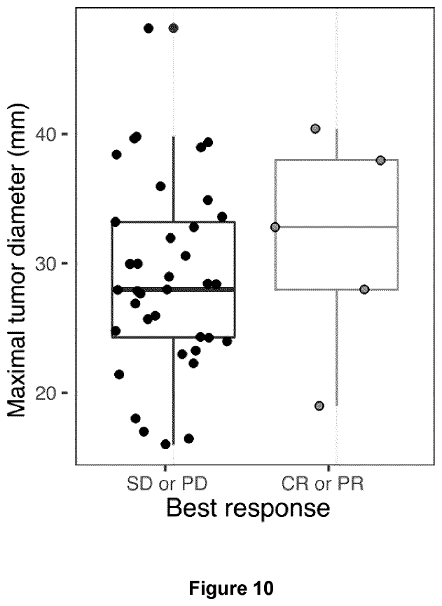

Patients with objective responses did not universally harbor characteristics that are commonly described in prognostically favorable tumors (Table 4). All patients with objective responses had reported IDH1 wildtype tumors by immunohistochemistry, and only 2 of them had had tumors with MGMT promoter hypermethylation. Additional targeted sequencing revealed that two patients with objective responses harbored mutations in either IDH1 or IDH2 at low allelic frequencies. Three of the patients with objective responses only had prior radiation and chemotherapy without prior resection of their tumor. The median tumor diameter was similar in patients with and without objective response (32.8 mm, 95% CI 25.2 to 46.6 versus 28.4 mm, 95% CI 24.8 to 30.8; FIG. 10).

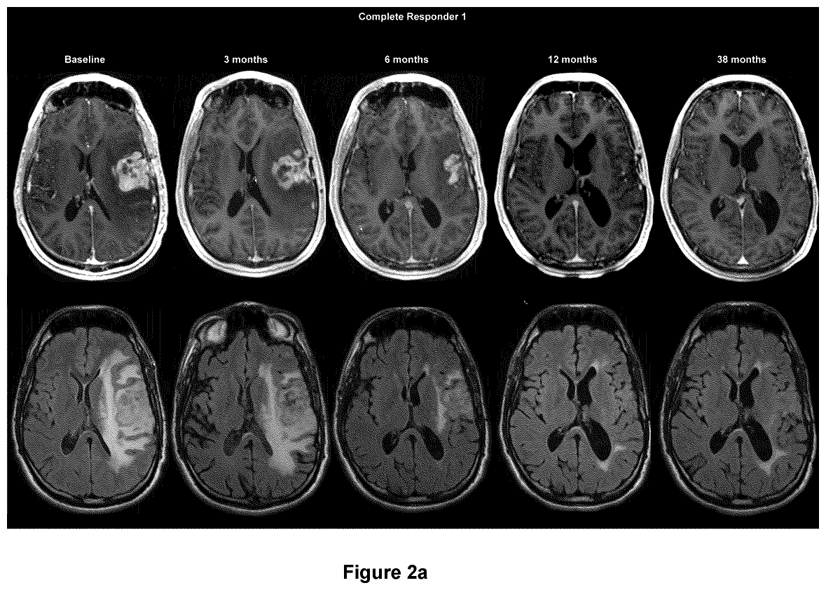

The two patients with complete response each had over 80% reduction in tumor volume approximately 6 months after treatment, that reached complete response criteria by 15-18 months after treatment (FIG. 2a and FIG. 2b). These two patients completed two-year treatment with pembrolizumab with durable responses and remain alive without evidence of disease progression.

### Survival Analyses

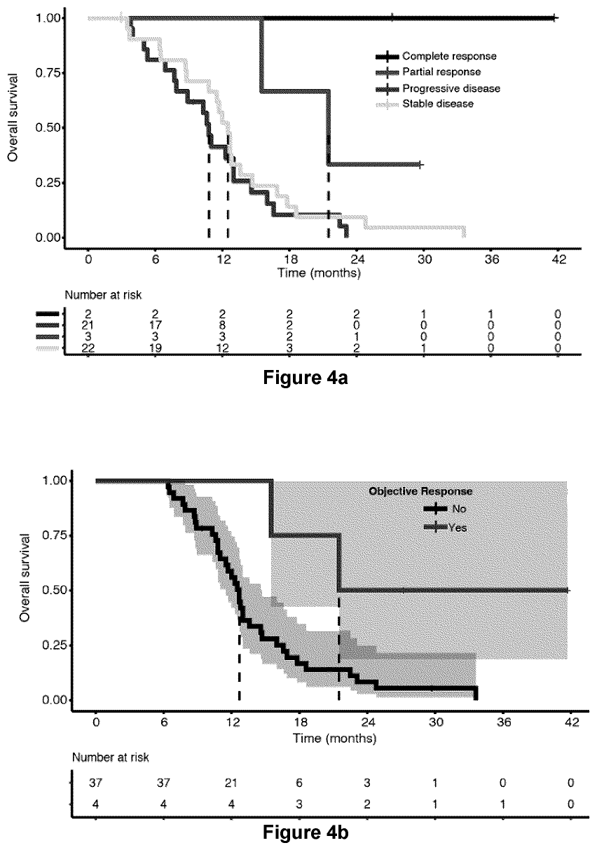

The secondary efficacy endpoint of 12-month survival was met. The 12-month overall survival was 52.7% (95% CI 40.1 to 69.2) in the intent-to-treat population and 53.1% (95% CI 36.8 to 67.0) in patients who received the declared dose of DNX-2401 (FIG. 1d), and this was greater than the prespecified threshold of 20% from an approved treatment approach. The median overall survival was 12.5 months (10.7 to 13.5) in the intent-to-treat population and 12.5 months (95% CI 10.2 to 13.0) in declared dose population. Patients with objective responses had longer survival than patients without objective responses that was statistically significant (HR 0.20, 95% CI 0.05 to 0.87, p=0.02; FIG. 4b). Three patients, all with objective responses (including the two patients with complete response) completed the prespecified pembrolizumab treatment and remain alive at the time this disclosure was written, beyond the study interval, at 45, 48 and 60 months. As well, one patient, with an IDH1 wildtype and MGMT unmethylated tumor received a total of 6 doses of pembrolizumab with overall stable disease. This patient elected to discontinue participation in the study and remained alive over 34 months after initiation of treatment.

### Exploratory Associations

The present inventors considered that concurrent use of medications may have impacted outcomes. Physicians were permitted to use low-dose bevacizumab or corticosteroids to address cerebral edema in this trial. Baseline corticosteroid use and corticosteroid use throughout the study were not statistically associated with outcomes, though use of corticosteroids throughout the study approached the threshold for statistical significance in some instances (Table 8). As well, none of the patients with an objective response received bevacizumab during treatment.

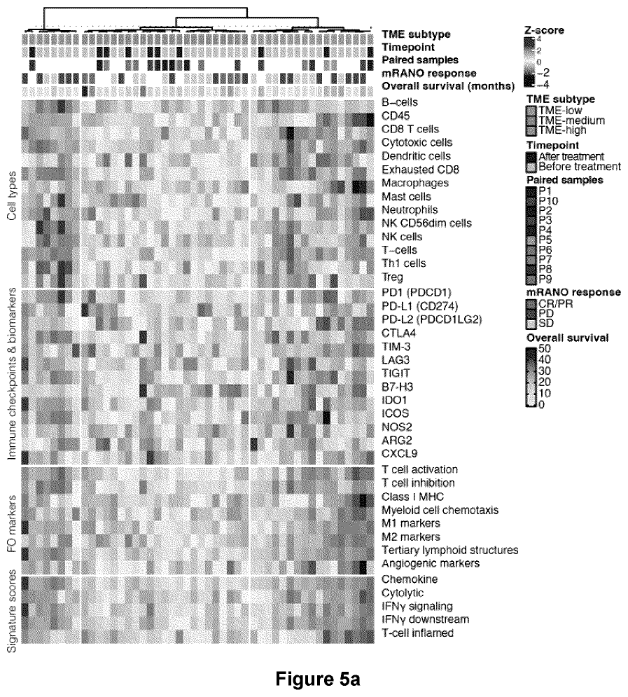

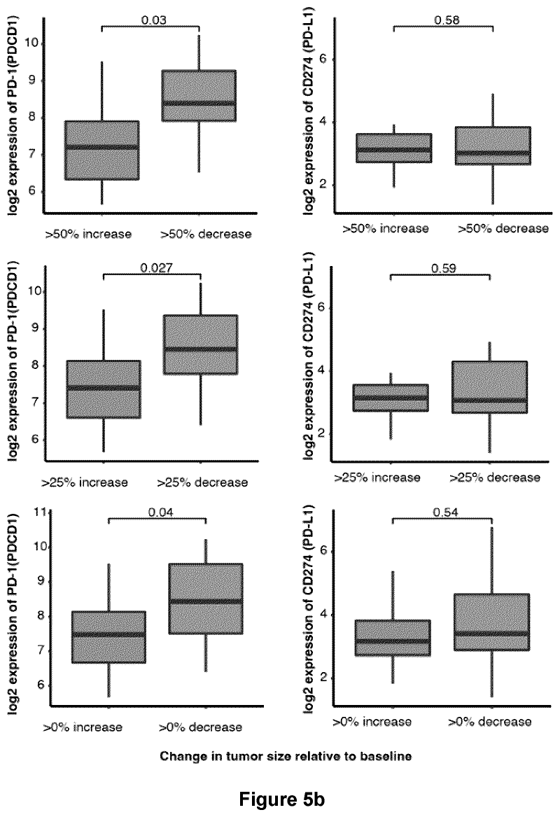

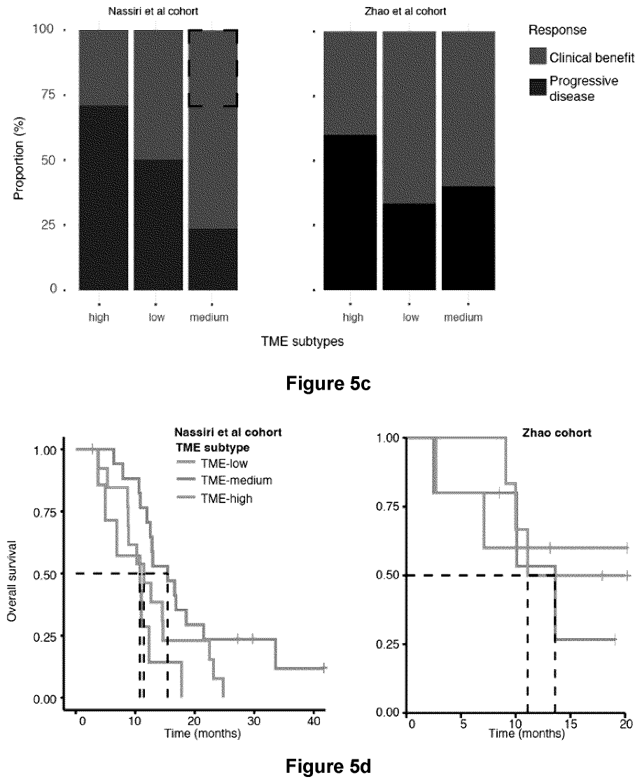

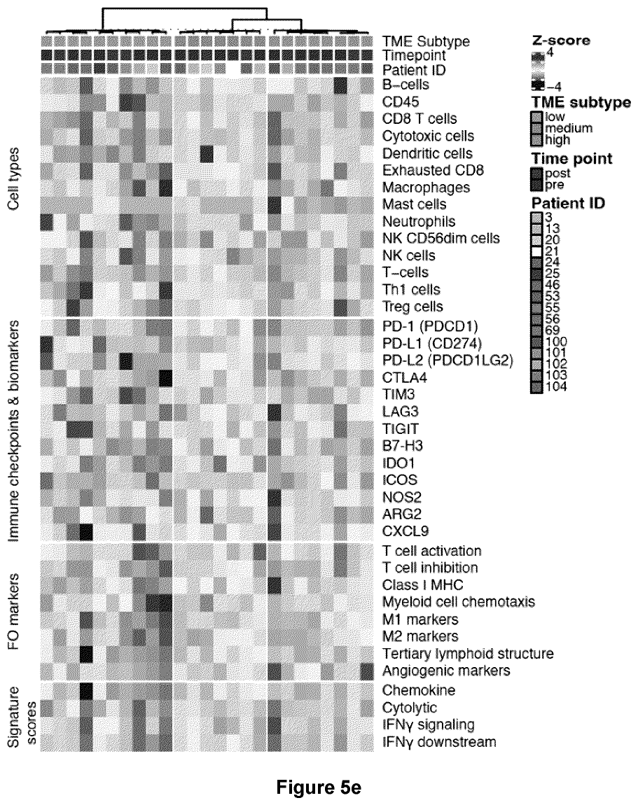

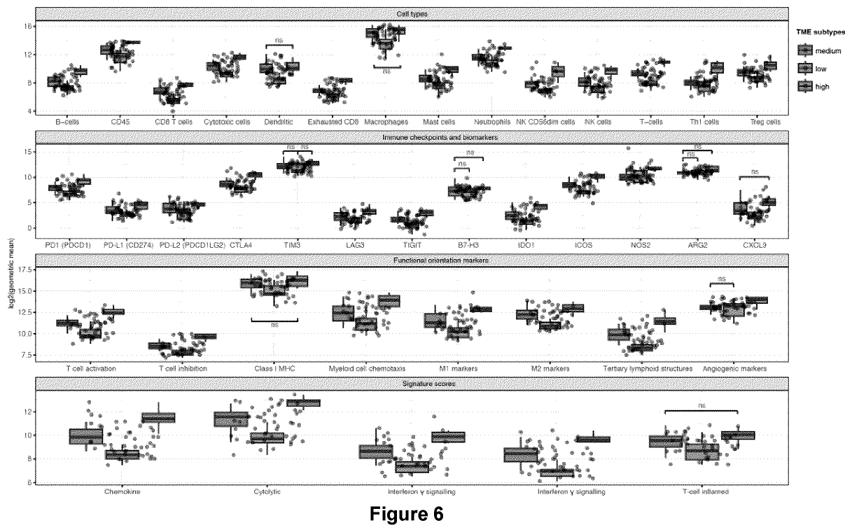

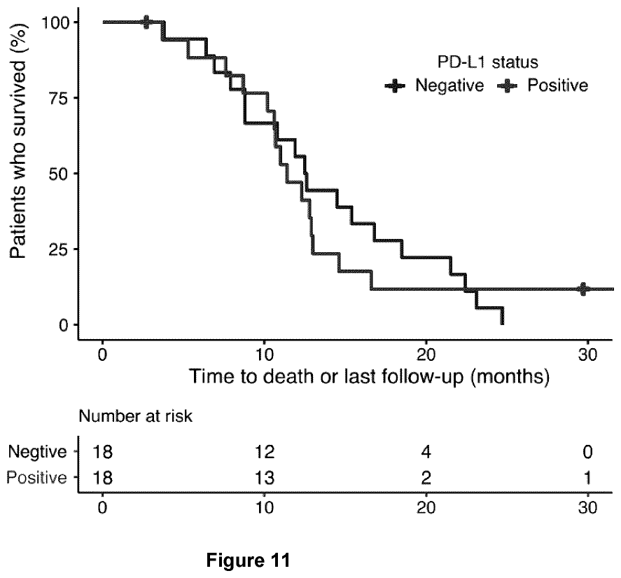

The present inventors also considered that variability in intrinsic patient and tumor factors might be associated with differences in outcomes of patients. To characterize potential biomarkers of treatment response, gene expression data were obtained on 38 patients with biopsy specimens available before treatment. The tumors from this study were divided into three tumor microenvironment subtypes (TMEhigh, TMEmedium, TMElow) on the basis of the degree of immune cell enrichment (FIG. 5a), as recently described in White, K. et al. Identification, validation and biological characterization of novel glioblastoma tumour microenvironment subtypes: Implications for precision immunotherapy. Ann. Oncol. (2022), herein incorporated by reference. TMEhigh tumors had high scores for multiple different immune cells but also highly expressed multiple complementary suppressive immune checkpoints genes (FIG. 6). By contrast, TMElow tumors had low immune cell scores with low expression of immune checkpoint genes. TMEmedium tumors had intermediary immune cell scores and expression of PDCD-1 (gene that encodes PD-1) but relatively low expression of other checkpoint proteins. The present inventors found that pre-treatment gene expression levels of PDCD-1, but not CD274 (gene that encodes PD-L1), was statistically significantly associated with reduction in tumor size (FIG. 5b and FIG. 11). All of the patients who had an objective response had TMEmedium tumors prior to treatment (29.4%, 95% CI 10.3 to 55.6%; P=0.012). Patients with TMEmedium tumors were more likely to have clinical benefit from treatment (OR=4.08, 95% CI 1.02 to 19.4, p=0.036; FIG. 5c), and also had statistically significantly longer survival in the cohort (HR 2.27, 95% CI 1.09 to 4.49, p=0.027; FIG. 5c). Patient samples from a prior trial investigating adjuvant anti-PD-1 monotherapy in recurrent glioblastoma¹² were also divisible into the same three TME subtypes, with less clear associations with outcomes (FIG. 5c and FIG. 5e).

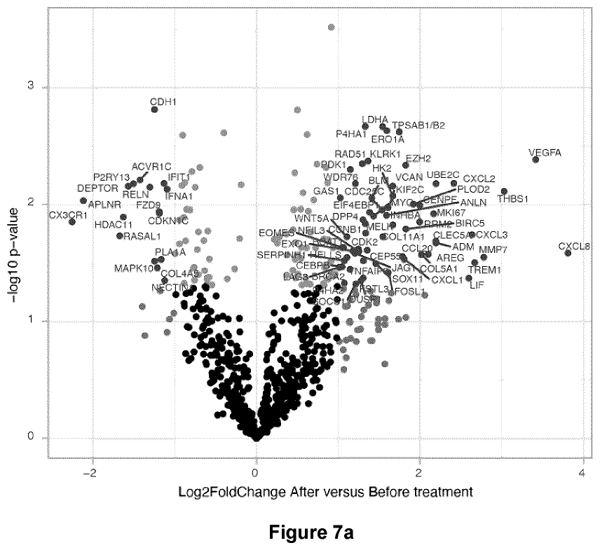

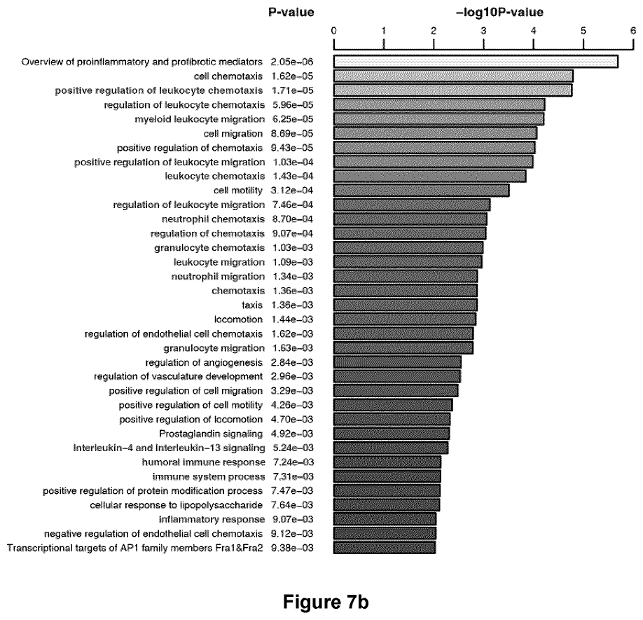

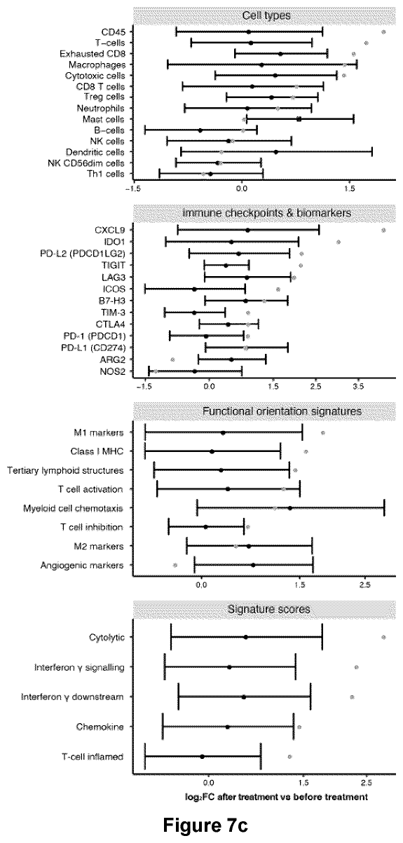

Ten patients also had biopsy specimens at the time of disease progression after treatment allowing for a biological assessment of matched-pair tissues. Of these 10 patients, one initially had a partial response to treatment prior to progression, while the other 9 patients did not demonstrate objective responses (3 patients with progressive disease as best response and 6 patients with initially stable disease as best response). Comparing gene expression profiles at disease progression after treatment to those at baseline before treatment revealed several differentially expressed genes (FIG. 7a). Genes that were overexpressed in post-treatment specimens were highly enriched for pathways involved in immune system activation and regulation by functional enrichment analysis (FIG. 7b). The patient with a partial response to treatment showed heightened immune activity after treatment relative to other patients, with the highest levels of interferon gamma and downstream signaling, infiltration of T-cells, as well as the highest score for a T-cell inflamed microenvironment (FIG. 7c)¹³. As well, the expression of several different immune checkpoint genes such as TIGIT (Log₂FC=1.77), LAG3 (Log₂FC=2.05) and CD276 (Log2FC=2.06) were consistently increased in post-treatment samples, and this was highest for the patient with a partial response to treatment.

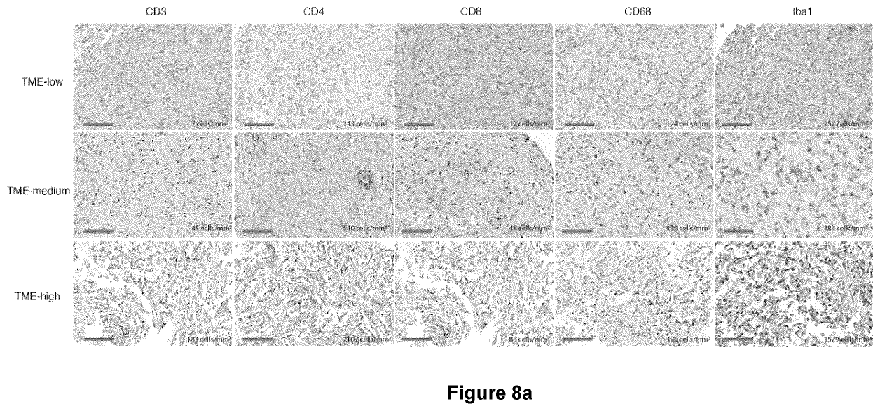

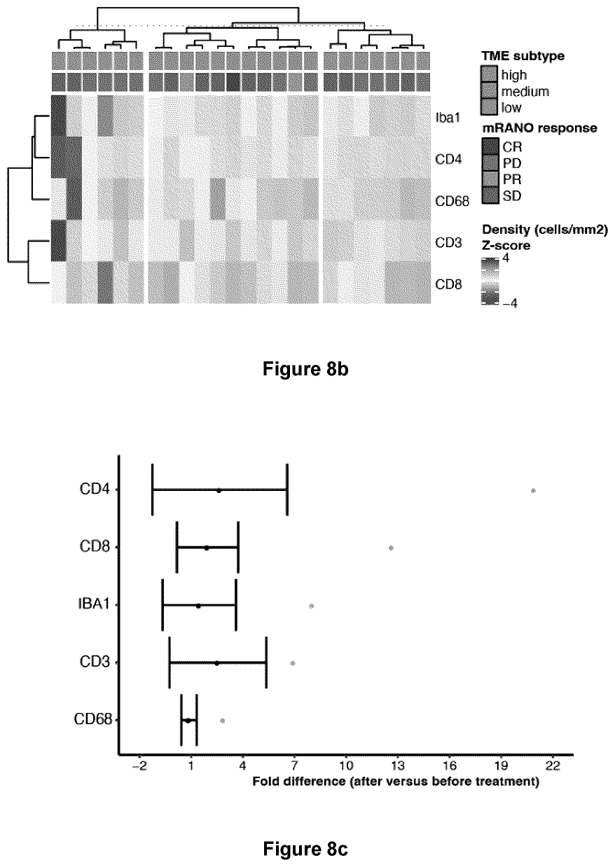

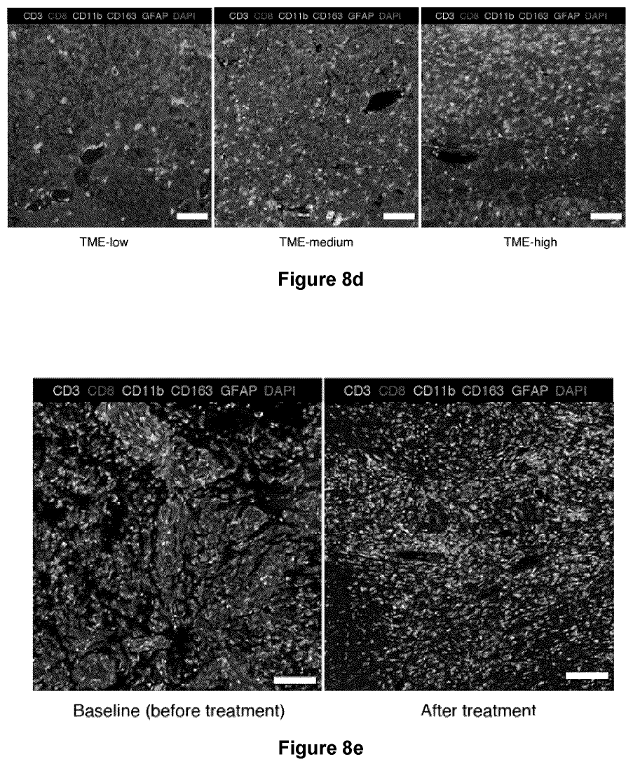

The present inventors performed immunophenotypic characterization of tumors before and after treatment by blinded immunohistochemical and multiplex immunofluorescent analysis. Patients with TME-medium and TME-high tumors by gene expression subtyping also showed progressively greater density of immune cell infiltrates by immunohistochemistry and immunofluorescence (FIG. 8a,FIG. 8b, and FIG. 8d). Comparing specimens before and after treatment, it was found that increases in density of microglia (Iba1), macrophages (CD68) and lymphocytes (CD3, CD4, CD8) after treatment was most evident in the patient who showed an objective response to treatment (FIG. 8c and FIG. 8e).

Certain pathogenic mutations are potentially associated with prognosis and specific response to checkpoint inhibition in glioblastoma¹⁴. Clinically relevant molecular features were reported by investigators for tumor biopsies analyzed using various assays at each clinical site. Investigators reported MGMT status, IDH1/2 mutation, and for 42 of 49 subjects, pathogenic mutations. Targeted NGS was also separately performed on available tumor biopsies on a subset of patients. A significant number of pathogenic mutations, including those in TP53, NF1, PTEN, MTOR, and RBI were detected, as were a few mutations in POLE and POLDI. There was no clear association between these specific molecular features, including tumor mutational burden, on response to treatment (Table 4 and Table 12).

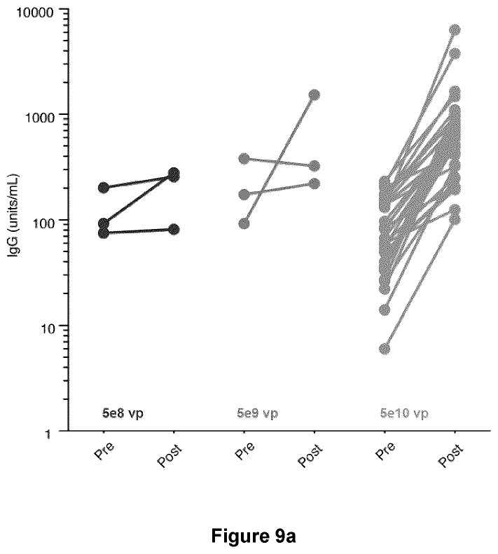

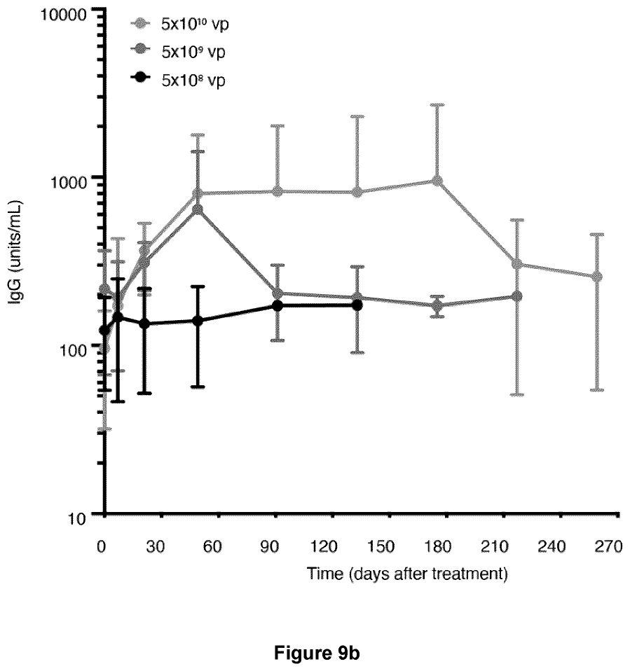

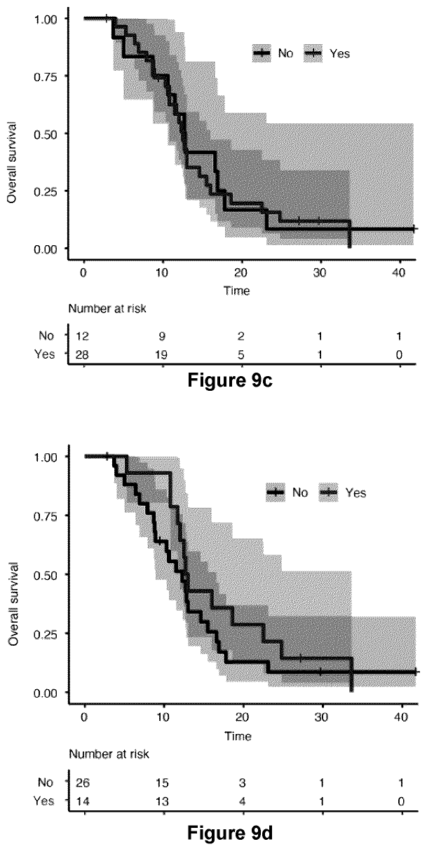

Anti-adenovirus antibodies were measured by direct immunofluorescence assay in the serum of patients before treatment and throughout the course of the trial. All patients were seropositive for IgG antibodies against adenoviral hexon protein before treatment with DNX-2401, and in general, anti-adenovirus IgG levels increased within 2 months post treatment, with levels sustained longest in patients treated with 5e10 vp DNX-2401, compared to lower doses (FIG. 9a and FIG. 9b). The present inventors considered that variability in systemic immunogenic response to DNX-2401 might have impacted outcomes. The median overall survival of patients with and without a systemic immunogenic response to DNX-2401 delivery, which was defined as a greater than 4-fold increase in baseline levels of anti-adenovirus antibodies, were similar (12.5 months, 95% CI 10.8 to 15.9 versus 12.8 months, 95% CI 10.6 to not reached). These findings were unchanged using more stringent thresholds of greater than 10-fold increase in baseline levels of anti-adenovirus antibodies (12.9 months, 95% CI 12.0 to not reached versus 12.3 months, 95% CI 8.9 to 16.6; FIG. 9c and FIG. 9d)

### DISCUSSION

Glioblastoma is a devastating disease and recurrence of disease is inevitable after initial treatment with radiotherapy and concurrent and adjuvant temozolomide chemotherapy. At progression, treatment options are very limited and of marginal efficacy. Immune checkpoint blockade in other advanced solid cancers such as melanoma¹⁵⁻¹⁷ and non-small cell lung cancer^{18,19} has greatly improved outcomes. However, the innately immunosuppressive microenvironment in glioblastomas has presumably rendered immune checkpoint blockade less effective for this disease^{4,5}.

DNX-2401 (Delta-24-RGD) is a conditionally replicative oncolytic adenovirus with a 24 base pair deletion in the ELA gene that renders selective replication of the virus in malignant cells with defective retinoblastoma signaling. DNX-2401 also has an RGD peptide insertion into the fibre-knob that allows the virus to anchor directly to integrins and improve the infectability of glioblastoma cells⁹. Preclinical studies of DNX-2401 in glioma mouse models showed promising antitumor immune activity as early as 1-2 weeks after delivery of a single dose of virus with potential for longer-term antigen-specific memory responses^{9,20}. This led to the first in human trials of DNX-2401 for glioblastoma, where in addition to direct oncolytic effects, it was shown that the delivery of the virus into tumors induced an immunogenic environment with increased T-cell infiltration and also altered the expression of checkpoint proteins¹⁰.

Treatment with oncolytic virus and immune checkpoint blockade combines the initial local effects of the oncolytic virus on the tumor microenvironment with the systemic effects of innate and adaptive immune responses from virus replication and PD-1 inhibition⁷. This combination has led to improved outcomes in other tumors, such as melanoma⁶, pointing to the possibility for therapeutic benefit of combination therapy in glioblastoma. Systematic screening of co-signaling molecules after DNX-2401 treatment in preclinical glioma models revealed significant increases in PD-1 expression that would prime the immune system for effective synergy with subsequent anti-PD-1 therapy²¹. Indeed, combination therapy of a single intratumoral dose of DNX-2401 followed by systemic pembrolizumab 1 week after viral treatment improved survival compared to monotherapy with either virus or pembrozliumab alone in glioma mouse models, providing rationale for further investigation in humans²¹.

Herein, the present inventors reported the results of a 2-part, phase 1/2, multicenter, open-label clinical trial evaluating the safety and efficacy of combined intratumoral delivery of DNX-2401 with systemic pembrolizumab for patients with recurrent glioblastoma treated at 13 institutions in North America. All centers used purpose-built cannulas to standardize the delivery of virus into the tumor eliminating backflow and ensuring full administration of virus to the tumor. A total of 48 of 49 patients successfully received treatment with DNX-2401 and pembrolizumab.

Between 5×10⁸ to 5×10¹⁰ viral particles of DNX-2401 were tested when delivered sequentially with pembrolizumab and it was found that the safety profile was consistent with prior studies reporting on oncolytic viruses or immunotherapies for brain tumors^{3,10,22}. There were no dose limiting toxicities in the dose escalation phase of this study, and no deaths that were directly related to the treatment regimen. The most common serious adverse event reported was neurological symptoms related to increase in peritumoral inflammation (cerebral edema), which occurred in 16% of patients. The possibility for treatment-induced cerebral edema was anticipated when designing this study due to inflammatory responses observed in Phase 1 study of DNX-2401 monotherapyl0, and so the present inventors allowed for a short-course steroid or low-dose bevacizumab regimen to mitigate these effects. All serious cerebral edema events were resolved with anticipated medical measures and surgical intervention to remove tumor due to tissue swelling was not necessary for any patient. The time-course of edema development in this trial was established by serial volumetric analysis of changes in perilesional FLAIR signal on imaging. Increases in volume of edema were found as early as 8 weeks after treatment that was sustained to 20 weeks, even in patients who did not become symptomatic with cerebral edema. This data can help inform on the expected time interval of cerebral edema for future trials of immunotherapy in recurrent glioblastoma. The non-neurologic toxicity profile in this study was otherwise comparable to those previously reported for pembrolizumab⁵.

In total, 5 patients had objective responses, with two patients showing durable complete responses >45 months and three patients remaining alive at the writing of this disclosure. The objective response rate was 10.4% (90% CI 4.2-20.7). It is noteworthy that there was one additional patient who received the declared dose of DNX-2401 with complete response at the site of treatment, however, this patient developed a new lesion at a distant site resulting in a classification of progressive disease. This patient remained alive a total of 12.3 months after treatment. In the previous phase 1 trial evaluating DNX-2401 monotherapy in recurrent glioma, there was also one patient with a complete response who developed a distant nodule several years after treatment¹⁰. Pathological examination of the nodule after resection showed only necrosis and inflammation without evidence of tumor. Although the patient in the present trial did not undergo resection for the new nodule, it is possible that the radiographic changes seen reflect a similar adaptive memory antitumor response that was observed in the original phase 1 trial of DNX-2401 monotherapy, and not progressive disease. Beyond this, prior reports of durable responses to immunotherapies have largely been limited to patients with favorable biological characteristics²³. Patients with objective responses in the present study had tumors that did not universally harbor the prognostically favorable mutation in IDH1 and had both MGMT methylated and unmethylated tumors, representing the group of glioblastomas that desperately need efficacious therapies.

The median overall survival was 12.5 months (10.7 to 13.5) and overall survival at 12 months was 52.7% (95% CI 40.1 to 69.2), which was greater than the prespecified threshold of 20% using approved treatment of tumor-treating fields by Novo-TTF²⁴. The 12-month overall survival was 32% in patients treated with DNX-2401 alone¹⁰, while median overall survival was at 9.3 months and 9.8 months with DNX-2401 or PD-1 blockade alone in prior trials^{5,10}. Although this trial was not designed to distinguish the effects of DNX-2401 versus pembrolizumab versus combination therapy, the notable survival data point to the potential of improved efficacy in combining oncolytic virus with checkpoint inhibition.

While the use of bevacizumab may complicate response assessment in trials by inducing changes in contrast enhancement seen on imaging, none of the patients with objective responses received bevacizumab during the study. As well, baseline corticosteroid use was not found to be associated with outcomes in the study, confirming the findings in a prior study evaluating neoadjuvant checkpoint blockade in recurrent glioblastoma²⁷. Without wishing to be bound by theory, this may be explained by the fact that patients using greater than 4 mg/day of dexamethasone as baseline were excluded from both studies. Although associations of steroid use throughout this study and outcomes were not statistically significant, some comparisons approached the threshold for significance.

Matched mutational data and gene expression data were obtained on tumor specimens from patients, where available. Three of the patients with objective responses (60%) had tumors with mutational burden (TMB) greater than 10 mutations/Mb, while two patients with objective responses (40%) had tumors with TMB less than 10 mutation/Mb. Although TMB is a known predictive biomarker of response to checkpoint inhibition in a range of advanced cancers, this relationship is more complex and has been less consistent in prior investigations in glioblastomas²⁸. One of the major determinants linking TMB to response to checkpoint inhibition is alterations in mismatch repair proteins or polymerase E and D (POLE and POLD) genes²⁸. None of the patients who showed objective responses had mutations in POLE or POLD genes. Without wishing to be bound by theory, this suggests that the antitumor responses after combined oncolytic virus and checkpoint inhibition in glioblastomas may be less dependent on TMB than in other solid cancers.

Using gene expression data, objective responses exclusively occurred in patients with moderately inflamed microenvironment, and modest PD-1 expression (TME^{medium}) before treatment (29.4%, 95% CI 10.3 to 55.6%). Clinical benefit rates and overall survival was also longer in TME^{medium} tumors in this trial. These findings are consistent with prior investigations and the present inventors' own findings that show that adjuvant anti-PD-1 inhibition does not improve survival in TME^{high} tumors^{11,27}. While TME^{high} tumors are enriched with immune cell infiltrates, they also highly express multiple different suppressive immune checkpoints leading to an exhaustive immune microenvironment by complementary mechanisms. TME^{medium} tumors are primed with a moderate degree of immune cells and express moderate levels of PD-1. DNX-2401 can induced further infiltration of cytotoxic T-cells and expression of PD-1 in these tumors that can be further targeted with subsequent anti-PD-1 treatment without immunosuppression from alternative checkpoint proteins. Specimens on disease progression were also obtained after treatment for 10 patients in this trial. The expression of several different immune checkpoints such as TIGIT, LAG3, and B7-H3 were elevated after treatment, pointing to the potential for using multiple parallel immune checkpoint inhibitors in TME^{medium} tumors that eventually develop disease progression. A similar approach could potentially be considered for TME^{high} tumors.

While by strict definition the primary endpoint of objective response was not met, the secondary endpoint of 12-month survival, which is more clinically meaningful and reliable than response rate, was met and the survival of objective responders are encouraging and, without wishing to be bound by theory, suggest that tumor control led to improved survival. Emerging data since the conception of this study has shown some potential benefit with multiple doses of oncolytic virus²⁹, suggesting that multiple doses of DNX-2401 with pembrolizumab may be beneficial, given the local immune-stimulatory effects of treatment.

### Methods

### Patients

Adult patients with histologically confirmed glioblastoma or gliosarcoma, presenting with documented failure of previous surgical resection, chemotherapy, and/or radiation at first or second recurrence, with a Karnofsky performance score of at least 70, were eligible. All patients were required to have a single contrast-enhancing tumor of at least 1 cm in two planes but no more than 4 cm in any single plane, as assessed by magnetic resonance imaging (MRI). Surgical resection must not have been possible or planned as part of the treatment for their presentation and the tumor must have been accessible for stereotactic delivery of DNX-2401. The full inclusion and exclusion criteria are detailed below.

### Inclusion Criteria:

1.≥18 years of age on the day of informed consent
2. A single glioblastoma or gliosarcoma tumor confirmed by documented historical histopathology.
   1. First or presenting second recurrence of glioblastoma or gliosarcoma (i.e., relapse following prior treatment) at time of consent. Approval may be given by Medical Monitor or designee to proceed with enrollment with a prior non-GBM/GS diagnosis, in which case transition to GBM/GS may be accepted as first recurrence of tumor.
   2. Gross total or partial tumor resection, including tumor debulking, is not possible or not planned
   3. A single measurable tumor that is at least 10.0 mm longest diameter (LDi)×10.0 mm shortest diameter (SDi) and that does not exceed 40.0 mm in LDi or SDi on the Screening MRI
   4. The measurable area of the tumor is solid/nodular and is not cystic
   5. Willing to provide a stereotactic biopsy sample from the brain tumor obtained prior to DNX-2401 administration
   6. Tumor must be accessible for stereotactic injection
   7. Evidence of tumor recurrence (e.g., progression after last treatment) on the Screening MRI (15 days to 72 hours prior to DNX-2401 administration)
   8. Tumor location that will not risk delivery of DNX-2401 into the ventricular system
3. Tumor recurrence or progression after previously failing surgical resection, chemotherapy or radiation
4. Resolution of toxic effect(s) of the most recent prior chemotherapy to Grade 1 or less (except neuropathy and alopecia)
5. Demonstrate adequate organ function as defined below:
   Hematological
      1. Absolute neutrophil count (ANC)≥1,500 cells/mm³
      2. WBC≥2.5×10³ cells/mm³
      3. Platelets≥100,000 cells/mm³
      4. Hemoglobin≥10 g/dL or ≥5.6 mmol/L
      5. Absolute lymphocyte count (ALC)≥800 cells/mm³
   Renal
      1. Creatinine≤1.5×ULN
      2. BUN≤1.5×ULN
   Hepatic
      1. Total bilirubin≤1.5×upper limit of normal (ULN)
         1. Note: In the event that total bilirubin is ≤1.5×ULN, the subject may be eligible if the direct bilirubin level is ≤ULN, following consultation with the DNAtrix Medical Monitor or designee.
      2. AST (SGOT) and ALT (SGPT)≤2.5×ULN
   Coagulation
      1. International Normalized Ratio (INR)≤1.5×ULN
      2. Prothrombin Time (PT)≤1.5×ULN
      3. Activated Partial Thromboplastin Time (aPTT)≤1.5×ULN
6. Adequate venous access
7. Karnofsky performance status≥70%
8. Afebrile at baseline/Day 0 prior to DNX-2401 administration (i.e., <38.0° C.)
9. Prior anti-tumor therapies must have been completed within the following time periods prior to DNX-2401 injection:
   2 weeks after vincristine
   4 weeks after nitrosoureas
   3 weeks after procarbazine or temozolomide
   4 weeks after bevacizumab, other antibody therapy or other anti-angiogenic therapy to treat glioblastoma
   5 half-lives for other anti-cancer agents or 2 weeks after the last dose when the half-life is unknown. A discussion of these agents will take place with the DNAtrix Medical Monitor or designee prior to establishing eligibility.
10. For applicable screening candidates, external beam radiotherapy (>5000 cGy) must have been completed at least 12 weeks prior to DNX-2401 administration
11. Females who are not of childbearing potential must be documented as such and will not be tested for pregnancy or required to utilize contraception if they meet one or more of the following definitions of non-childbearing potential:
   Amenorrheic for >2 years without a hysterectomy and bilateral oophorectomy and a FSH value in the postmenopausal range upon pre-trial (screening) evaluation
   Post-hysterectomy, bilateral oophorectomy or tubal ligation. Tubal ligation must be confirmed with medical records of the actual procedure.
12. Female subjects of childbearing potential must have a negative urine or serum pregnancy test within 24 hours prior to receiving DNX-2401 injection. If the urine test is positive or cannot be confirmed as negative, a serum pregnancy test will be required to confirm negative results. The serum pregnancy test must be negative for the subject to be eligible.
13. Female subjects of childbearing potential must be willing to use two highly effective birth control methods throughout the study, starting with provision of informed consent through 180 days after the single dose of DNX-2401 and 120 days after the last dose of pembrolizumab. The two birth control methods can be either two barrier methods or a barrier method plus a hormonal method to prevent pregnancy. Examples of highly effective birth control methods include the following:
   Using twice the normal protection of birth control (i.e., double-barrier) by using a condom AND spermicidal jelly or foam, or a diaphragm AND spermicidal jelly or foam. A spermicidal jelly or foam must be used in addition to a barrier method (e.g., condom or diaphragm)
   Oral contraceptive control pills
   Depot or injectable birth control
   Intrauterine Device (IUD)
   Transdermal contraceptive patch
   Vaginal contraceptive ring
14. Male subjects must agree to use an acceptable method of contraception throughout the study starting with provision of informed consent through 180 days after the single dose of DNX-2401 and 120 days after the last dose of pembrolizumab.
15. Willing and able to provide informed consent, undergo and comply with all study assessments and adhere to the protocol schedule
16. Agree not to donate blood or gametes following DNX-2401 administration

### Exclusion Criteria:

Subjects who meet any of the following exclusion criteria are not eligible for the study and must not be enrolled:
1. Recurrent GBM with multiple (>2) separate enhancing tumors (measurable or non-measurable)
2. Tumor shape that is bi-lobular or multifocal tumor
3. Tumor involvement that would require ventricular, brainstem or posterior fossa injection or access through a ventricle or risk of ventricular penetration in order to deliver DNX-2401
4. Tumor involves both hemispheres or there is suspected cerebrospinal fluid (CSF) dissemination
5. Documented extracranial metastases
6. Requires, or based upon historical evidence, may require treatment with high-dose systemic corticosteroids defined as dexamethasone>4 mg/day or bioequivalent for more than 3 consecutive days within 2 weeks prior to and following the first dose of pembrolizumab, or has demonstrated an inability to be tapered off of steroids
7. Uncontrolled blood-sugar levels defined as HbAlc>7%
8. Active autoimmune disease that requires, or has required, systemic treatment in the past 2 years (i.e. with use of disease modifying agents, corticosteroids or immunosuppressive drugs)
9. Previous treatment with any checkpoint inhibitor (e.g., anti-PD-1, anti-PD-L1, or anti-PD-L2 agent) or with an agent directed to another stimulatory or co-inhibitory T-cell receptor (e.g., CTLA-4, OX-40, CD137), including pembrolizumab
10. History of (non-infectious) pneumonitis that required steroids or current pneumonitis
11. History of interstitial lung disease
12. Transfusions or medications (e.g., G-CSF) to treat pancytopenia or other hematological conditions within 4 weeks prior to DNX-2401 administration
13. Prior gene transfer therapy or prior therapy with cytolytic virus of any type
14. Live vaccines of any kind within 45 days prior to DNX-2401 administration and while participating in the study. Examples of live vaccines include, but are not limited to, the following: measles, mumps, rubella, varicella/zoster (chicken pox), yellow fever, rabies, BCG, and typhoid vaccine. Seasonal influenza vaccines for injection are generally killed virus vaccines and are permitted; however, intranasal influenza vaccines (e.g. Flu-Mist®) are live attenuated vaccines and are not allowed.
15. Major surgery within 4 weeks and minor surgery within 2 weeks of DNX-2401 administration (Refer to Appendix 2-Major and Minor Surgery Definitions)
16. Participation in an investigational (invasive device, drug, or product) study or treatment with an investigational agent or device within 30 days prior to consent
17. Any contraindication for undergoing MRI such as: individuals with pacemakers, epicardial pacer wires, infusion pumps, surgical and/or aneurysm clips, shrapnel, metal prosthesis, implants with potential magnetic properties, or metallic bodies in the eyes
18. Is pregnant or breastfeeding, or planning to conceive or father children during the study, starting with the screening visit through 180 days after the single dose of DNX-2401 and 120 days after the last dose of pembrolizumab
19. Evidence of active uncontrolled infection or an unstable or severe intercurrent medical condition that requires treatment and/or precludes surgery
20. History of prior malignancy except for curatively treated basal or squamous cell carcinoma of the skin (non-melanoma skin cancer), cervical or vaginal intra-epithelial neoplasia, non-invasive breast cancer in situ or localized prostate cancer with a prostate specific antigen (PSA) of <4.0 ng/ml (mcg/L) at Screening. Subjects with other curatively treated malignancies who had no evidence of metastatic disease and a >2 year disease-free interval may be enrolled after approval by the DNAtrix Medical Monitor or designee.
21. Any medical condition that precludes intratumoral injection into the brain
22. Immunocompromised subjects or those with autoimmune conditions, human immunodeficiency virus (HIV), or active hepatitis (according to diagnostic serology results that are positive for active HAV, HBV [Anti-HBc and HBsAg] or HCV infection)
23. Pulmonary conditions including a known history of active tuberculosis (TB, Mycobacterium tuberculosis). TB testing is required for subjects recently exposed to persons with active TB or who have traveled recently to areas where TB is endemic.
24. Evidence of bleeding diathesis, hemorrhage, or coagulopathy or use of anticoagulant medication or any medication that may increase the risk of bleeding that cannot be stopped prior to surgery. If the medication can be discontinued prior to DNX-2401 injection, then the subject may be eligible following consultation with the DNAtrix Medical Monitor or designee.
25. Encephalitis, multiple sclerosis or other central nervous system (CNS) infection or primary CNS disease that would interfere with subject evaluation
26. Li-Fraumeni Syndrome or with a known germ line deficit in the retinoblastoma gene or its related pathways
27. Significant systemic or major illnesses including, but not limited to, congestive heart failure, ischemic heart disease, kidney disease or renal failure, organ transplantation or other conditions that may affect subject risk or protocol compliance
28. Alcohol or substance abuse or alcohol dependency within 12 months prior to screening that has caused health consequences
29. History or current diagnosis of any medical or psychological condition, and in particular, any unstable CNS condition such as delirium, confusion, etc., that might interfere with the subject's ability to comply with the study requirements or the ability to obtain informed consent

### Design

To evaluate the safety of combining DNX-2401 with pembrolizumab, an initial dose-escalation phase was conducted to determine a safe dose of DNX-2401 in combination with pembrolizumab and followed by a dose-expansion phase. All patients received a single dose of DNX-2401 by stereotactic injection at the time of standard tumor biopsy followed by 200 mg pembrolizumab infused intravenously at a dose of 200 mg over 30 minutes every 3 weeks starting 7 days after DNX-2401. Resection of tumors was not permitted. Treatment with pembrolizumab continued for up to 2 years, or until one of the following occurred: disease progression, unacceptable toxic effects, or withdrawal of consent. Dose-escalation evaluated 5×10⁸, 5×10⁹, and 5×10¹⁰ viral particles (vp) DNX-2401 in combination with standard dosing pembrolizumab in a 3+3 design.

All patients underwent a stereotactic biopsy to document the presence of tumor tissue prior to delivery of DNX-2401. Immediately after biopsy, a stereotactic-compatible neuro-ventricular cannula (Alcyone MEMS, Lowell MA; ClearPoint SmartFlow, Irvine CA) was inserted into the tumor to deliver the precise targeted dose of DNX-2401 via a single micro-tip at a rate of 0.9 mL per hour over approximately 1 hour. The cannula was left in place for 10 minutes after administration of virus to allow viral particles to diffuse without backflow prior to removal.

### Assessments

Patients were continuously monitored throughout the study for safety as outlined in the schedule of assessments in the study Protocol. Adverse events and serious adverse events were graded according to National Cancer Institute-Common Terminology Criteria for Adverse Events, version 4.03, and their relationship to treatment administered was assessed. For the dose-escalation phase, the dose-limiting toxicity window of observation was the first 21 days after initial pembrolizumab infusion. The occurrence of any of the following toxicities is considered a DLT, if judged by the Investigator to be possibly, probably or definitely related to administration of DNX-2401 and pembrolizumab (and not to the administration procedure):
1. Grade 4 non-hematologic toxicity (not laboratory)
2. Grade 4 hematologic toxicity lasting≥7 days
3. Grade 3 non-hematologic toxicity (not laboratory) lasting>3 days despite optimal supportive care
4. Any Grade 3 or Grade 4 non-hematologic laboratory value if:
   Medical intervention is required to treat the subject, or
   The abnormality leads to hospitalization, or
   The abnormality persists for >1 week
5. Febrile neutropenia Grade 3 or Grade 4:
   Grade 3 is defined as ANC<1000/mm³ with a single temperature of >38.3° C. (101° F.) or a sustained temperature of ≥38° C. (100.4° F.) for more than one hour
   Grade 4 is defined as ANC <1000/mm³ with a single temperature of >38.3° C. (101° F.) or a sustained temperature of ≥38° C. (100.4° F.) for more than one hour, with life-threatening consequences and urgent intervention indicated
6. Thrombocytopenia<25,000/mm³ if associated with:
   A bleeding event which does not result in hemodynamic instability but requires an elective platelet transfusion, or

Treatment response was determined by serial protocolized contrast-enhanced MRI every 4 weeks for 28 weeks, and afterward at an interval of every 8 weeks for the remainder of the treatment period. Patients who completed the treatment phase entered the long-term response and survival follow-up phase of the study for the rest of life, with MRI every 16 weeks. Objective responses were evaluated by the RANO criteria^{30,31} and modified RANO (mRANO) criteria³². Complete and partial responses required confirmation on the consecutive scan 4 weeks after the initial response was observed. Patients with suspected radiological progression were permitted to remain on study until progression was confirmed by follow-up MRI separated by a minimum of 4 weeks.

### Endpoints and Statistical Analyses

The analyses reported herein were performed according to the statistical analysis plan. All enrolled patients were included in the safety analysis set and patients were considered evaluable for efficacy if they received at least one dose, or part of one dose, of either study drug, had measurable tumor at baseline and completed the week 4 follow-up visit. Patients who discontinued study participation for any reason other than progressive disease or study treatment-related toxicity prior to the 4-week visit were not considered evaluable and were replaced, however, they continued to be monitored for safety.

The primary safety objective was to evaluate the safety of escalating doses of DNX-2401 and the overall safety of the declared dose of intratumoral DNX-2401 when followed by sequential intravenous administration of pembrolizumab. Adverse events and serious adverse events were summarized for all patients in the study and were considered treatment-related if reported as possibly, probably, or definitely related to study drug.

The primary efficacy objective was to determine the objective response rate, defined as the percentage of patients that had complete or partial responses based on mRANO criteria^{30,31}. The primary endpoint was tested in a single arm design. The sample size estimation was based on a prespecified historical response rate of 5%, with alpha=0.05, a total of 39 evaluable subjects in the declared dose phase would yield an 80% power for an alternative hypothesis of objective response rate of 18%. Objective response rate was reported as the number and percentage of subjects with an objective response and the corresponding 95% CI based on the exact binomial method (Clopper-Pearson Method). Type I error was set at 5% (one-sided), so it was predetermined that the 90% CI would also be provided. Secondary efficacy objectives were to evaluate 12-month overall survival as well as the clinical benefit rate, defined as the proportion of patients treated with DNX-2401 and pembrolizumab who had stable disease, complete response, or partial response. Overall survival was defined as the time from the start of treatment (DNX-2401 injection) until death (or last follow-up). Overall survival at 12 months was summarized using Kaplan-Meier methods and outcomes were compared to historical rates of 20% from an approved treatment approach, NovoTTF²⁴. Overall survival of patients with objective responses was compared to those without objective responses using 6-month landmark Kaplan-Meier method to account for potential lead time bias³³. IDH1 mutation status and MGMT methylation status were assessed locally at each institution. Follow-up of survival for patients remaining alive after database lock were used for descriptive purposes only.

### Study Organization and Oversight

The study was conducted in compliance with the Protocol at 15 clinical trial sites in the United States and Canada, as well as recognized international standards including the Good Clinical Practice guidelines of the International Conference on Harmonisation and the principles of the Declaration of Helsinki. The Protocol and its amendments were approved by the institutional review board of each participating trial site. Voluntary written informed consent was obtained from every patient prior to participation in this study. DNX-2401 preparation, handling and administration followed institutional standards for Biosafety Level 2 agents.

### Anti-Adenovirus Antibodies

Anti-hexon IgG antibody levels were determined before and after treatment by ELISA from patient serum samples according to manufacturer's instructions (Adenovirus IgG ELISA Kit; DEIA309; Creative Diagnostics). Absorbance at 450 nm was measured using a Synergy H4 plate reader (BioTek), and concentrations calculated based on a standard curve (Gen 5 software Version 3.0, BioTek). Anti-adenovirus IgG serum concentration increases of 4-fold or greater were considered seroconversions. A more stringent threshold of 10-fold or greater increases in levels of Anti-adenovirus IgG serum concentrations was also tested.

### Targeted Mutational Sequencing

Targeted next-generation sequencing (NGS) was performed on DNA extracted from formalin-fixed, paraffin-embedded pretreatment tumor biopsies available from 28 patients. Tumor samples from 18 subjects were sequenced by NeoGenomics using NeoType Discovery Profile for Solid Tumor. Tumor samples from 10 subjects were sequenced by NovoGene Co. using Novogene PM 2.0.

### Gene Expression Profiling and Analyses

RNA was extracted from formalin-fixed, paraffin-embedded pretreatment tumor biopsies available from 38 patients and analyzed retrospectively on the NanoString nCounter system. For 10 patients, there was also tumor biopsy specimens available at the time of disease progression, allowing for an examination of gene expression changes before and after treatment in matched patient samples.

The geometric mean of canonical marker genes was used to compute scores for immune cell types³⁴, functional orientation markers, and signature scores reported herein, unless otherwise explicitly stated. Functional orientation markers and the chemokine and cytolytic signature scores were obtained from previous studies^{11,35,36}. Remaining marker genes are provided in Table 13. A T-cell-inflamed signature was computed as previously described using a weighted sum of normalized expression values of 18 inflammatory genes (CCL5, CD27, CD274 [PD-L1], CD276 [B7-H3], CD8A, CMKLR1, CXCL9, CXCR6, HLA.DQA1, HLA.DRB1, HLA.E, IDO1, LAG3, NKG7, PDCDILG2 [PD-L2], PSMB10, STAT1, and TIGIT) related to antigen presentation, chemokine expression, cytolytic activity, and adaptive immune resistance¹³. Glioblastoma microenvironment subtypes were obtained by partition-around-medoid clustering using immune cell type scores, as previously described¹¹. Differentially expressed genes between groups were identified by comparing Log₂ fold change and Welch's P-values. Genes with absolute value Log₂FC>1 and P<0.05 were considered differentially expressed, unless otherwise specified. Functional enrichment analysis was performed using gProfiler.

### Previously Published Datasets

Zhao et al. previously published their transcriptomic data in patients receiving anti-PD-1 therapy in high grade gliomas¹². A total of 16 patients had transcriptomic data available prior to initiation of anti-PD-1 therapy and 9 patients also had transcriptomic data available at progression after initiating anti-PD-1 therapy. The transcriptomic data from these 25 patients were downloaded from SRAPRJNA482620 and clinical annotation was provided by the authors. Response was considered as stable disease or better in this study. Associations with outcome were based on overall survival after initiating anti-PD-1 therapy.

### Edema Volumetric Analysis

Digital Imaging and Communications in Medicine (DICOM) files for study MRIs were imported into Horos (version 3.3.6), and a blinded reviewer used non-motion degraded, axial, fluid attenuated inversion recovery (FLAIR) sequences to segment perilesional FLAIR hyperintense signal. The Horos volume generator function was used to determine the total FLAIR signal volume for each study MRI. Volume of edema at each study MRI was normalized relative to baseline levels. Grouped comparisons were made by calculating the mean normalized edema volume with 95% confidence intervals at the timepoints outlined in the protocol every 4 weeks for 28 weeks and then every 8 weeks thereafter.

### Immunohistochemistry

Immunohistochemical analyses were performed for myeloid cell markers (Iba-1, CD68, CD163) and lymphoid cell markers (CD3, CD4, CD8) in samples with available tissue before and after treatment in this sample. Staining and subsequent annotation and analyses were performed blinded to clinical status. Slides with 5-micron FFPE tissue sections were rehydrated and a sodium citrate-dihydrate buffer or Tris-EDTA buffer was used for heat-mediated antigen retrieval. A 3% hydrogen peroxide in methanol solution was utilized to block endogenous peroxidase activity. Blocking solution (5% bovine serum albumin in phosphate buffered saline plus 0.1% Triton X-100) was applied to slides for 1 hour at room temperature. Subsequently, primary antibodies including anti-CD3 (Agilent, M725401-2, mouse monoclonal, 1:100), anti-IBA1 (Wako, 019-19741, rabbit polyclonal, 1:1500), anti-CD68 (Agilent, M0514, mouse monoclonal, 1:200), anti-CD4 (abcam, ab133616, rabbit monoclonal, 1:100) and anti-CD8 (abcam, ab93278, rabbit monoclonal, 1:250) were applied overnight at 4° C. in blocking solution. A 1 h incubation with secondary antibody was performed followed by processing with the DAKO polymer-HRP system and DAB peroxidase kit, counterstaining with hematoxylin, dehydration of the tissue and coverslipping. Whole slide images were digitized and then for each slide tumor versus non tumor content was annotated and representative images were selected. Proportions of stain positive cells were quantified using HALO (version 3.0311, Indica Labs, Albuquerque, NM, USA) software algorithms that were defined to identify cells with either nuclear or cytoplasmic staining as a fraction of all cells. This algorithm was applied to all annotated tissue sections in an unbiased systematic manner, and the density of immunopositivity per mm² was recorded for each antibody. PD-L1 protein expression was performed by NeoGenomics Laboratories Inc. (NeoGenomics) under the direction of Merck using formalin-fixed paraffin embedded (FFPE) tumor biopsy samples according to standard protocols (PD-L1 IHC 22C3 assay).

### Multiplex Immunofluorescence Staining, Tissue Imaging, and Cell Phenotyping

A validated and standardized multiplex immunofluorescence protocol was developed for simultaneous detection of CD3, CD8, CD11b, CD163, GFAP, and DAPI in a single formalin-fixed paraffin-embedded (FFPE) tissue section. The validation pipeline for the multiplex immunofluorescence protocol has been previously described⁸. Briefly, whole-slide tissue sections were deparaffinized and subjected to sequential rounds of antibody staining. Antigen retrieval was performed using Dako PT-Link heat-induced antigen retrieval with low pH (pH6) or high pH (pH9) target retrieval solution (Dako). The antibody panel included CD11b (Rabbit monoclonal, clone EPR1344, 1:1000, Abcam, product number ab133357), CD163 (Mouse monoclonal, clone MRQ-26, ready-to-use, Cell Marque, product number 760-4437), CD3 (Rabbit polyclonal, IgG, ready-to-use, Agilent, product number IR503), CD8 (Mouse monoclonal, clone C8/144B, ready-to-use, Agilent, product number IR623), and GFAP (Mouse monoclonal, clone 6F2, 1:500, Agilent, product number M0761). After all sequential rounds, nuclei were counterstained with spectral DAPI (Akoya Biosciences) and sections were mounted with Faramount Aqueous Mounting Medium (Dako).

Multiplexed immunofluorescence slides were scanned on a Vectra-Polaris Automated Quantitative Pathology Imaging System (Akoya Biosciences). Spectral unmixing was performed using inForm software (version 2.4.8, Akoya Biosciences), as described. Image analysis was performed using QuPath and Fiji/ImageJ. Briefly, cells were segmented based on nuclear detection using the StarDist 2D algorithm. A random trees algorithm classifier was trained for each cell marker. Cells were then subclassified as CD3+, CD8+, CD11b+, and CD163+ cells. CD4+ T-cells were defined as CD3+CD8−. Cells negative for these markers were defined as “other cell types”. Measurements were calculated as cell densities (cells/mm²). GFAP was used identified tumor areas.

While the present disclosure has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### Tables

TABLE 1
Patient Demographics and Baseline Characteristics

| | |
|---|---|
| | Patients (N = 49), |
| Characteristic | No. (%) |

| | |
|---|---|
| Median age (range)-yr. | 53 (26-73) |

Sex

| | |
|---|---|
| Female | 20 (41%) |
| Male | 29 (59%) |

Diagnosis at enrollment

| | |
|---|---|
| Glioblastoma | 48 (98%) |
| Gliosarcoma | 1 (2%) |

Karnofsky Performance Score

| | |
|---|---|
| 100 | 10 (20%) |
| 90 | 28 (57%) |
| 80 | 6 (12%) |
| 70 | 5 (10%) |

Recurrences prior to treatment

| | |
|---|---|
| 1 | 39 (80%) |
| 2 | 10 (20%) |

Baseline tumor size

| | |
|---|---|
| Median maximum diameter (range)-mm | 28.5 (11.0-48.2) |
| Median tumor area (range)-mm² | 597.2 (110.0-1599.8) |

IDH1 R132 status

| | |
|---|---|
| Mutant | 4 (8%) |
| Wildtype | 44 (90%) |
| Unknown | 1 (2%) |

MGMT gene promoter methylation status

| | |
|---|---|
| Methylated | 14 (29%) |
| Unmethylated | 28 (57%) |
| Unknown | 7 (14%) |

Prior therapies

| | |
|---|---|
| Surgical resection | 44 (90%) |
| Radiotherapy | 49 (100%) |
| Temozolomide | 49 (100%) |
| Bevacizumab | 6 (12%) |
| Tumor treating fields | 5 (10%) |

Baseline dexamethasone use ≥1.5 mg/day

| | |
|---|---|
| Yes | 9 (18%) |
| No | 40 (82%) |

TABLE 2
Summary of adverse events related to treatment

| | | | | | |
|---|---|---|---|---|---|
| Grade 1 | Grade 2 | Grade 3 | Grade 4 | Grade 5 | Total |
| n (%) | n (%) | n (%) | n (%) | n (%) | n (%) |

AE related to treatment

| | | | | | | | | | | |
|---|---|---|---|---|---|---|---|---|---|---|
| Overall | 6 | (12%) | 16 | (33%) | 11 | (22%) | 1 (2%) | 0 (0%) | 34 | (69%) |
| Frequency | | | | | | | | | | |
| Brain edema | 2 | (4%) | 8 | (16%) | 7 | (14%) | 1 (2%) | 0 (0%) | 18 | (37%) |
| Headache | 4 | (8%) | 9 | (18%) | 2 | (4%) | 0 (0%) | 0 (0%) | 15 | (31%) |
| Fatigue | 7 | (14%) | 7 | (14%) | 0 | (0%) | 0 (0%) | 0 (0%) | 14 | (29%) |
| Dysphasia | 4 | (8%) | 4 | (8%) | 0 | (0%) | 0 (0%) | 0 (0%) | 8 | (16%) |
| Hemiparesis | 0 | (0%) | 2 | (4%) | 4 | (8%) | 0 (0%) | 0 (0%) | 6 | (12%) |
| Pyrexia | 5 | (10%) | 0 | (0%) | 0 | (0%) | 0 (0%) | 0 (0%) | 5 | (10%) |
| Decreased | 3 | (6%) | 1 | (2%) | 0 | (0%) | 0 (0%) | 0 (0%) | 4 | (8%) |
| appetite | | | | | | | | | | |
| Myalgia | 2 | (4%) | 2 | (4%) | 0 | (0%) | 0 (0%) | 0 (0%) | 4 | (8%) |
| Nausea | 2 | (4%) | 2 | (4%) | 0 | (0%) | 0 (0%) | 0 (0%) | 4 | (8%) |

SAE related to treatment

| | | | | | | | | | | |
|---|---|---|---|---|---|---|---|---|---|---|
| Overall | 0 | (0%) | 4 | (8%) | 9 | (18%) | 1 (2%) | 0 (0%) | 14 | (29%) |
| Frequency | | | | | | | | | | |
| Brain/vasogenic | 0 | (0%) | 1 | (2%) | 6 | (12%) | 1 (2%) | 0 (0%) | 8 | (16%) |
| edema | | | | | | | | | | |
| Dysphasia | 0 | (0%) | 3 | (6%) | 0 | (0%) | 0 (0%) | 0 (0%) | 3 | (6%) |
| Hemiparesis | 0 | (0%) | 0 | (0%) | 3 | (6%) | 0 (0%) | 0 (0%) | 3 | (6%) |

AE denotes adverse event; SAE denotes serious adverse event. Shown are adverse events and serious adverse events with greater than 5% frequency. Overall frequency refers to patients reporting at least one treatment related adverse event. Each patient is included once using the highest-grade event. Events were graded according to National Cancer Institute-Common Terminology Criteria for Adverse Events, version 4.03.

TABLE 3
Summary of efficacy endpoints

| | Intent to treat population, N = 48 | Declared dose cohort, N = 42 |
|---|---|---|
| Response, % | | |
| Objective response rate | 10.4% (3.5 to 22.3; 4.2 to 20.7) | 11.9% (4.8 to 23.4; 3.9 to 25.6) |
| (90% CI; 95% CI) | | |
| CR | 4.1% (0.5 to 14.2; 0.7 to 12.5) | 4.8% (0.9 to 14.2; 0.6 to 16.1) |
| PR | 6.3% (1.3 to 17.1; 1.7 to 15.3) | 7.1% (1.9 to 17.4; 1.5 to 19.4) |
| SD (95% CI) | 45.8% (31.3 to 60.8) | 42.9% (27.7 to 59.0) |
| PD (95% CI) | 43.8% (29.4 to 58.8) | 45.2% (29.8 to 61.3) |
| Clinical benefit rate (95% CI) | 56.2% (41.1 to 70.5) | 54.8% (38.7 to 70.2) |
| Survival | | |
| 12-month overall survival | 52.7% (40.1 to 69.2) | 53.1% (36.8 to 67.0) |
| (95% CI) | | |
| Overall survival (95% CI) | 12.5 months (10.8 to 14.6) | 12.5 months (10.2 to 13.0) |

CR denotes complete response; PR denotes partial response, SD denotes stable disease, PD denotes progressive disease.
The primary efficacy endpoint was objective response rate, and the two secondary efficacy endpoints were clinical benefit rate and 12-month overall survival.

TABLE 4
Baseline characteristics of patients with complete or partial responses per mRANO criteria

| | 2401013 | 2401039 | 2401045 | 2401047 | 2401019 |
|---|---|---|---|---|---|
| Response to | CR | PR | CR | PR | PR |
| treatment | | | | | |
| Maximum reduction in | 100 | 72.2 | 100 | 63.6 | 78.1 |
| tumor volume from | | | | | |
| baseline | | | | | |
| PFS (mo) | 41.6+ | 5.3 | 26.8+ | 3.7 | 26.9 |
| Survival (mo.) | 41.6 (alive) | 21.5 | 26.8 (alive) | 15.4 | 29.7 (alive) |
| Age-yrs | 26 | 50 | 27 | 39 | 51 |
| Recurrences (No.) | 1 | 1 | 2 | 1 | 2 |
| Prior Resection | No | Total | Total | No | No |
| | | resection | resection | | |
| Baseline steroids | Yes | Yes | No | No | No |
| IDH1 R132H mutation | wildtype* | wildtype | wildtype | wildtype | wildtype** |
| status (IHC) | | | | | |
| MGMT promoter | unknown | unmethylated | methylated | unknown | methylated |
| methylation status | | | | | |
| Maximal tumor | 40.4 | 32.8 | 38 | 19 | 28 |
| diameter (mm) | | | | | |
| Pre-treatment PD-L1 | unknown | negative | unknown | negative | positive |
| expression by | | | | | |
| immunohistochemistry | | | | | |
| Tumor Mutational | 5.7 | 7.8 | 10.0 | 7.8 | 10.0 |
| Burden (non | | | | | |
| synonymous | | | | | |
| mutations per | | | | | |
| 10 megabases) | | | | | |

CR denotes complete response;
PR denotes partial response;
+patients remain without progression at last follow-up;
*IDH1 wildtype, but IDH2 mutation detectable by sequencing at allelicf requency of 23%,
**IDH1 R132H wildtype by IHC, but detectable by sequencing at allelic frequency of 13%

TABLE 5
Summary of adverse events

| | | | Cohort 3/ | |
|---|---|---|---|---|
| | | | Dose | |
| | Cohort 1 | Cohort 2 | expansion | |
| | 5 × 10⁸ vp | 5 × 10⁹ vp | 5 × 10¹⁰ vp | |
| | DNX-2401, | DNX-2401, | DNX-2401, | Total, |
| | n = 4 | n = 3 | n = 42 | n = 49 |
| Treatment | 4 (100%) | 3 (100%) | 42 (100%) | 49 (100%) |
| emergent AE | | | | |
| Grade 1 | 0 (0%) | 0 (0%) | 3 (7%) | 3 (6%) |
| Grade 2 | 2 (50%) | 0 (0%) | 12 (29%) | 14 (29%) |
| Grade 3 | 2 (50%) | 4 (100%) | 24 (57%) | 29 (59%) |
| Grade 4 | 0 (0%) | 0 (0%) | 2 (5%) | 2 (4%) |
| Grade 5 | 0 (0%) | 0 (0%) | 1 (2%) | 1 (2%) |
| Treatment | 0 (0%) | 2 (67%) | 26 (62%) | 28 (57%) |
| related AE | | | | |
| Treatment | 3 (75%) | 1 (33%) | 21 (50%) | 25 (51%) |
| emergent SAE | | | | |
| Grade 1 | 0 (0%) | 0 (0%) | 0 (0%) | 0 (0%) |
| Grade 2 | 2 (50%) | 1 (33%) | 4 (10%) | 7 (14%) |
| Grade 3 | 1 (25%) | 0 (0%) | 15 (36%) | 16 (33%) |
| Grade 4 | 0 (0%) | 0 (0%) | 1 (2%) | 1 (2%) |
| Grade 5 | 0 (0%) | 0 (0%) | 1 (2%) | 1 (2%) |
| Treatment | 0 (0%) | 1 (33%) | 11 (26%) | 12 (25%) |
| related SAE | | | | |
| Death due to an | 0 (0%) | 0 (0%) | 1 (2%) | 1 (2%) |
| AE | | | | |
| Death due to a | 0 (0%) | 0 (0%) | 0 (0%) | 0 (0%) |
| treatment- | | | | |
| related AE | | | | |
| AE leading to | 1 (25%) | 0 (0%) | 1 (2%) | 2 (4%) |
| premature | | | | |
| treatment | | | | |
| withdrawal | | | | |

AE denotes adverse event, SAE denotes serious adverse event; frequency refers to patients reporting at least one adverse event (each patient is includedonce), treatment related include those classified as possibly, probably, or definitely related to pembrolizumab + DNX2401.

TABLE 6
Summary of most commonly reported adverse events

| | | | Cohort 3/ | |
|---|---|---|---|---|
| | | | Dose | |
| | Cohort 1 | Cohort 2 | expansion | |
| | 5 × 10⁸ vp | 5 × 10⁹ vp | 5 × 10¹⁰ vp | |
| | DNX-2401, | DNX-2401, | DNX-2401, | Total, |
| | n = 4 | n = 3 | n = 42 | n = 49 |
| Subjects Reporting | 4 (100.0%) | 3 (100.0%) | 41 (97.6%) | 48 (98.0%) |
| at Least One Most- | | | | |
| Frequently Occurring | | | | |
| AE | | | | |
| Headache | 1 (25.0%) | 3 (100.0%) | 31 (73.8%) | 35 (71.4%) |
| Fatigue | 2 (50.0%) | 2 (66.7%) | 30 (71.4%) | 34 (69.4%) |
| Brain edema | 0 (0.0%) | 0 (0.0%) | 22 (52.3%) | 21 (44.9%) |
| Hemiparesis | 0 (0.0%) | 0 (0.0%) | 17 (40.5%) | 17 (34.7%) |
| Aphasia | 0 (0.0%) | 2 (66.7%) | 11 (26.2%) | 13 (26.5%) |
| Nausea | 0 (0.0%) | 2 (66.7%) | 10 (23.8%) | 12 (24.5%) |
| Seizure | 2 (50.0%) | 1 (33.3%) | 9 (21.4%) | 12 (24.5%) |
| Constipation | 0 (0.0%) | 1 (33.3%) | 8 (19.0%) | 9 (18.4%) |
| Decreased appetite | 0 (0.0%) | 2 (66.7%) | 7 (16.7%) | 9 (18.4%) |
| Memory impairment | 0 (0.0%) | 2 (66.7%) | 7 (16.7%) | 9 (18.4%) |
| Pyrexia | 0 (0.0%) | 1 (33.3%) | 8 (19.0%) | 9 (18.4%) |
| Gait disturbance | 1 (25.0%) | 1 (33.3%) | 6 (14.3%) | 8 (16.3%) |
| Muscular weakness | 0 (0.0%) | 1 (33.3%) | 7 (16.7%) | 8 (16.3%) |
| Vomiting | 0 (0.0%) | 1 (33.3%) | 7 (16.7%) | 8 (16.3%) |
| Depression | 1 (25.0%) | 2 (66.7%) | 4 (9.5%) | 7 (14.3%) |
| Fall | 0 (0.0%) | 1 (33.3%) | 6 (14.3%) | 7 (14.3%) |
| Myalgia | 0 (0.0%) | 0 (0.0%) | 7 (16.7%) | 7 (14.3%) |
| Pruritus | 0 (0.0%) | 1 (33.3%) | 6 (14.3%) | 7 (14.3%) |
| Arthralgia | 0 (0.0%) | 1 (33.3%) | 5 (11.9%) | 6 (12.2%) |
| Confusional state | 0 (0.0%) | 0 (0.0%) | 6 (14.3%) | 6 (12.2%) |
| Dizziness | 0 (0.0%) | 1 (33.3%) | 5 (11.9%) | 6 (12.2%) |
| Paraesthesia | 1 (25.0%) | 1 (33.3%) | 4 (9.5%) | 6 (12.2%) |
| Alanine | 0 (0.0%) | 0 (0.0%) | 5 (11.9%) | 5 (10.2%) |
| aminotransferase | | | | |
| increased | | | | |
| Insomnia | 0 (0.0%) | 1 (33.3%) | 4 (9.5%) | 5 (10.2%) |
| Lethargy | 0 (0.0%) | 0 (0.0%) | 5 (11.9%) | 5 (10.2%) |
| Somnolence | 0 (0.0%) | 0 (0.0%) | 5 (11.9%) | 5 (10.2%) |

AE denotes adverse event.
Listed are the most frequently reported AEs (≥10% Overall) by decreasing frequency of preferred term

TABLE 7
Subsequent therapies after trial termination

| | Declared dose | Intent-to-treat |
|---|---|---|
| | population | population |
| | Patients, | Patients, |
| Therapies | No. (%) | No. (%) |
| Any subsequent therapy | 30 (71) | 36 (75) |
| Surgery | 10 (24) | 13 (27) |
| Laser ablation | 1 (2) | 1 (2) |
| Radiotherapy | 9 (21) | 11 (23) |
| Systemic Therapy | | |
| Bevacizumab | 21 (50) | 23 (48) |
| Lomustine | 13 (31) | 17 (35) |
| Pembrolizumab | 7 (17) | 8 (16) |
| Temozolomide | 6 (14) | 7 (15) |
| Carboplatin | 2 (5) | 3 (6) |
| Everolimus | 2 (5) | 2 (5) |
| Imipridone | 2 (5) | 2 (5) |
| Depatuxizumab mafodotin | 2 (5) | 2 (5) |
| Procarbazine | 1 (2) | 1 (2) |
| Etoposide | 1 (2) | 1 (2) |
| Olaparib | 1 (2) | 1 (2) |
| Pabinostat | 1 (2) | 1 (2) |
| Irinotecan | 1 (2) | 1 (2) |
| Terameprocol | 1 (2) | 1 (2) |
| Debio1347 | 0 (0) | 1 (2) |
| Nivo | 0 (0) | 1 (2) |
| Dabrafenib | 0 (0) | 1 (2) |
| Trametinib | 0 (0) | 1 (2) |

TABLE 8
Corticosteroid use and outcomes

| | | | | Objective Response | | | Clinical Benefit | | |
|---|---|---|---|---|---|---|---|---|---|
| | Overall Survival | | | (CR + PR) | | | (CR + PR + SD) | | |
| | | 95% | P- | | 95% | P- | | 95% | P- |
| | HR | CI | value | OR | CI | value | OR | CI | value |
| Baseline | 1.72 | 0.58 to | 0.23 | 1.54 | 0.15 to | 0.72 | 0.53 | 0.10 to | 0.44 |
| steroid | | 4.2 | | | 16.2 | | | 2.68 | |
| (>2 mg/d | | | | | | | | | |
| versus <2 | | | | | | | | | |
| mg/d) | | | | | | | | | |
| Steroid | 1.81 | 0.98 to | 0.06 | 0.16 | 0.02 to | 0.12 | 0.32 | 0.10 to | 0.07 |
| throughout | | 3.3 | | | 1.59 | | | 1.08 | |
| study | | | | | | | | | |
| (>2 mg/d | | | | | | | | | |
| versus <2 | | | | | | | | | |
| mg/d) | | | | | | | | | |

TABLE 9
Summary of adverse events by counts

| | | | Cohort 3/ | |
|---|---|---|---|---|
| | | | Dose | |
| | Cohort 1 | Cohort 2 | expansion | |
| | 5 × 10⁸ vp | 5 × 10⁹ vp | 5 × 10¹⁰ vp | |
| | DNX-2401, | DNX- 2401, | DNX-2401, | Total, |
| | n = 4 | n = 3 | n = 42 | n = 49 |
| Total number | 26 | 78 | 672 | 776 |
| of treatment | | | | |
| emergent AE | | | | |
| Grade 1 | 8 | 41 | 389 | 438 |
| Grade 2 | 13 | 26 | 205 | 244 |
| Grade 3 | 5 | 11 | 73 | 89 |
| Grade 4 | 0 | 0 | 4 | 4 |
| Grade 5 | 0 | 0 | 1 | 1 |
| Total number | 5 | 1 | 43 | 49 |
| of treatment | | | | |
| emergent SAE | | | | |
| Grade 1 | 0 | 0 | 1 | 1 |
| Grade 2 | 3 | 1 | 11 | 15 |
| Grade 3 | 2 | 0 | 27 | 29 |
| Grade 4 | 0 | 0 | 3 | 3 |
| Grade 5 | 0 | 0 | 1 | 1 |

Shown are total counts of adverse events, AE denotes adverse event, SAE denotes serious adverse event

TABLE 10
Bevacizumab use during treatment phase.

| Subject | Start Date | End date | | | | |
|---|---|---|---|---|---|---|
| ID | (study day) | (study day) | Dose | Unit | Frequency | Indication |
| 2401007 | 54 | 54 | 735 | ml | once | Prophylaxis: edema |
| 2401023 | 32 | 60 | 5 | mg/kg | every 2 | Adverse event: vasogenic |
| | | | | | weeks | edema |
| 2401025 | 37 | 63 | 5 | mg/kg | every 2 | Adverse event: vasogenic |
| | | | | | weeks | edema |
| | 98 | 126 | 5 | mg/kg | every 2 | Adverse event: vasogenic |
| | | | | | weeks | edema |
| 2401026 | 43 | 70 | 3 | mg/kg | every 2 | Adverse event: vasogenic |
| | | | | | weeks | edema |
| | 103 | 147 | 5 | mg/kg | every 2 | Adverse event: vasogenic |
| | | | | | weeks | edema |
| 2401036 | 103 | 103 | 410 | mg | once | Adverse event: left sided |
| | | | | | | weakness |
| | 122 | 122 | 420 | mg | once | Adverse event: left sided |
| | | | | | | weakness |
| | 145 | 145 | 420 | mg | once | Adverse event: left sided |
| | | | | | | weakness |
| 2401037 | 82 | 82 | 340 | mg | once | Adverse event: vasogenic |
| | | | | | | edema |
| | 93 | 93 | 340 | mg | once | Adverse event: vasogenic |
| | | | | | | edema |
| | 109 | 109 | 340 | mg | once | Adverse event: vasogenic |
| | | | | | | edema |
| 2401038 | 60 | 60 | 330 | mg | once | Adverse event: vasogenic |
| | | | | | | edema |
| | 81 | 81 | 330 | mg | once | Adverse event: vasogenic |
| | | | | | | edema |
| | 102 | 102 | 330 | mg | once | Adverse event: vasogenic |
| | | | | | | edema |
| 2401040 | 54 | 54 | 5 | mg/kg | once | Adverse event: vasogenic |
| | | | | | | edema |
| | 71 | 71 | 5 | mg/kg | once | Adverse event: vasogenic |
| | | | | | | edema |
| | 85 | 85 | 5 | mg/kg | once | Adverse event: vasogenic |
| | | | | | | edema |
| | 97 | 97 | 10 | mg/kg | once | Adverse event: right sided |
| | | | | | | weakness |
| 2401041 | 75 | 75 | 5 | mg/kg | once | Adverse event: vasogenic |
| | | | | | | edema |
| | 96 | 96 | 5 | mg/kg | once | Adverse event: vasogenic |
| | | | | | | edema |
| | 173 | 173 | 10 | mg/kg | once | Adverse event: vasogenic |
| | | | | | | edema |
| 2401042 | 135 | 135 | 720 | mg | once | Adverse event: vasogenic |
| | | | | | | edema |
| | 149 | 149 | 370 | mg | once | Adverse event: vasogenic |
| | | | | | | edema |
| | 164 | 164 | 370 | mg | once | Adverse event: vasogenic |
| | | | | | | edema |
| 2401043 | 175 | 175 | 320 | mg | once | Other: Progression of |
| | | | | | | disease |
| | 189 | 189 | 320 | mg | once | Other: Progression of |
| | | | | | | disease |
| | 203 | 203 | 320 | mg | once | Other: Progression of |
| | | | | | | disease |
| | 280 | 280 | 640 | mg | once | Other: Progression of |
| | | | | | | disease |
| | 293 | 293 | 640 | mg | once | Other: Progression of |
| | | | | | | disease |
| | 306 | 306 | 320 | mg | once | Other: Progression of |
| | | | | | | disease |
| 2401048 | 52 | 52 | 385 | mg | once | Adverse event: vasogenic |
| | | | | | | edema |
| | 66 | 66 | 385 | mg | once | Adverse event: vasogenic |
| | | | | | | edema |
| | 80 | 80 | 385 | mg | once | Adverse event: vasogenic |
| | | | | | | edema |

TABLE 11
Details of serious adverse events attributable to study treatment

| | | | | | | | |
|---|---|---|---|---|---|---|---|
| | Dose | Days after | | | | | |
| Adverse | DNX- | DNX- | | | Effect on DNX- | | |
| Event | 2401 | 2401 | CTCAE | | 2401 or | | |
| Subject ID | (vp) | injection | Grade | Treatment | Pembrolizumab | Outcome | Sequalae |

Dysphasia

| | | | | | | | |
|---|---|---|---|---|---|---|---|
| 2401008 | 5 × 10⁹ | 29-41 | 2 | Steroids | None | Resolved | Yes |
| 2401025 | 5 × 10¹⁰ | 1-4 | 2 | None | None | Resolved | None |
| 2401029 | 5 × 10¹⁰ | 9-10 | 2 | Steroids | None | Resolved | None |

Headache

| | | | | | | | |
|---|---|---|---|---|---|---|---|
| 2401009 | 5 × 10¹⁰ | 17-24 | 2 | Concomitant medication | None | Resolved | None |
| 2401031 | 5 × 10¹⁰ | 10-12 | 2 | Concomitant medication | None | Resolved | None |

Edema

| | | | | | | | |
|---|---|---|---|---|---|---|---|
| 2401015 | 5 × 10¹⁰ | 24-28 | 3 | Steroids | Interrupted | Resolved | None |
| 2401018 | 5 × 10¹⁰ | 11-20 | 3 | Steroids | None | Resolved | None |
| 2401037 | 5 × 10¹⁰ | 65-66 | 3 | Concomitant medication | None | Resolved | Yes |
| | | | | and steroids | | | |
| 2401040 | 5 × 10¹⁰ | 53-55 | 3 | Concomitant medication | Interrupted | Resolved | Yes |
| | | | | and steroids | | | |
| 2401042 | 5 × 10¹⁰ | 132-135 | 4 | Concomitant medication | Interrupted | Resolved | None |
| 2401044 | 5 × 10¹⁰ | 39-44 | 3 | Steroids | None | Resolved | None |
| 2401044 | 5 × 10¹⁰ | 92-96 | 3 | Concomitant medication | Interrupted | Resolved | Yes |
| 2401047 | 5 × 10¹⁰ | 10-12 | 2 | Concomitant medication | None | Resolved | None |
| 2401048 | 5 × 10¹⁰ | 23-24 | 3 | Steroids | Discontinued | Resolved | Yes |

Hemiparesis

| | | | | | | | |
|---|---|---|---|---|---|---|---|
| 2401025 | 5 × 10¹⁰ | 1-4 | 3 | None | None | Resolved | None |
| 2401029 | 5 × 10¹⁰ | 9-10 | 3 | Steroids | None | Resolved | Yes |
| 2401048 | 5 × 10¹⁰ | 23-26 | 3 | Steroids | Discontinued | Resolved | Yes |

Autoimmune Colitis

| | | | | | | | |
|---|---|---|---|---|---|---|---|
| 2401022 | 5 × 10¹⁰ | 87-92 | 3 | Concomitant medication | Discontinued | Resolved | None |

Noninfectious Meningitis

| | | | | | | | |
|---|---|---|---|---|---|---|---|
| 2401045 | 5 × 10¹⁰ | 4-9 | 3 | Concomitant medication | None | Resolved | None |

Somnolence

| | | | | | | | |
|---|---|---|---|---|---|---|---|
| 2401048 | 5 × 10¹⁰ | 23-24 | 2 | Concomitant medication | Discontinued | Resolved | None |

TABLE 12
Molecular features with potential clinical relevance identified in pretreatment tumor biopsies

| DNX- | | | | | | | | Targeted Sequencing |
|---|---|---|---|---|---|---|---|---|
| 2401 | | | | | | | | Identified Molecular |
| dose | Subject | Best | Investigator Reported Molecular Features² | | | | | Features³ |
| (vp) | ID | Response | PD-L1¹ | MGMT | IDH1 | IDH2 | Other | Selected mutations |
| 5e8 | 2401001 | SD | POS | UNM | WT | WT | MUT = 1P | MUT = TP53 92% |
| | | | | | | | WT = 19Q | WT = IDH1, PTEN, RB1, |
| | | | | | | | | NF1, MTOR, POLE |
| | 2401003 | SD | POS | UNM | WT | WT | MUT = GFAP, Vimentin, EGFR, | MUT = PTEN 55% |
| | | | | | | | OTIG2, PhosphoH3 | WT = IDH1, RB1, TP53, |
| | | | | | | | KLE7 20%, TP53 rare positive cells | NF1, MTOR, POLE |
| | 2401049 | PD | NR | MET | WT | NR | MUT = EGFR, GFAP | WT = IDH1, PTEN, RB1, |
| | | | | | | | WT = KI67, P53, PTEN, VEGF | TP53, NF1, MTOR, |
| | | | | | | | | POLE |
| | 2401011 | PD | NEG | UNM | WT | WT | MUT = EGFR, GFAP | MUT = RB1 38% |
| | | | | | | | WT = EGFRVIII, KI67, P53 | WT = IDH1, PTEN, NF1, |
| | | | | | | | ATRX retained | TP53, MTOR, POLE |
| 5e9 | 2401002 | PD | POS | NR | MUT | NR | NR | MUT = IDH1 43% |
| | | | | | | | | WT = PTEN, RB1, TP53, |
| | | | | | | | | NF1, MTOR, POLE |
| | 2401008 | SD | NEG | UNM | WT | NR | MUT = P53, EGFR | MUT = TP53 69% |
| | | | | | | | | WT = IDH1, PTEN, RB1, |
| | | | | | | | | TP53, NF1, MTOR, |
| | | | | | | | | POLE |
| | 2401012 | SD | NR | MET | WT | NR | MUT = BRAF | MUT = POLE 24% |
| | | | | | | | | WT = IDH1, PTEN, RB1, |
| | | | | | | | | TP53, NF1, MTOR |
| 5e10 | 2401004 | PD | POS | NR | MUT | WT | MUT = ATRX, PDGFRA, RAD50, | MUT = IDH1 44%, PT53 |
| | | | | | | | RB1 loss, TP53 | 93% WT = RB1, TP53, |
| | | | | | | | | NF1, MTOR, POLE |
| | 2401005 | SD | NEG | UNM | WT | WT | MUT = CDK4, ERBB3, FRS2, | NR |
| | | | | | | | MDM2, SETD2 | |
| | 2401006 | SD | POS | MET | WT | NR | MUT = EGFR, SETD2, TERT | WT = IDH1, PTEN, RB1, |
| | | | | | | | MSS, TMB-LOW | TP53, NF1, MTOR, |
| | | | | | | | | POLE |
| | 2401007 | PD | NEG | UNM | WT | WT | MUT = EGFR, P53 | NR |
| | 2401009 | PD | NEG | UNM | WT | WT | MUT = EGFR (diffuse) | NR |
| | | | | | | | WT = P53 | |
| | 2401010 | PD | NR | UNM | WT | WT | MUT = EGFR, P53 | NR |
| | | | | | | | KI67 26% | |
| | 2401013 | CR | NR | NR | WT | NR | NR | MUT = NF1 16%, IDH2 |
| | | | | | | | | 23% |
| | | | | | | | | WT = IDH1, PTEN, RB1, |
| | | | | | | | | TP53, MTOR, POLE |
| | 2401014 | SD | POS | NR | WT | NR | MUT = P53 | MUT = PTEN 72%, |
| | | | | | | | ATRX retained | TP53 71%, MTOR 40% |
| | | | | | | | | WT = IDH1, RB1, NF1, |
| | | | | | | | | POLE |
| | 2401015 | PD | NR | MET | WT | NR | NR | WT = IDH1, PTEN, RB1, |
| | | | | | | | | TP53, NF1, MTOR, |
| | | | | | | | | POLE |
| | 2401016 | PD | POS | NR | WT | NR | NR | NR |
| | 2401017 | SD | POS | NR | WT | NR | NR | MUT = PTEN 21% |
| | | | | | | | | WT = IDH1, RB1, TP53, |
| | | | | | | | | NF1, MTOR, POLE |
| | 2401018 | PD | POS | UNM | WT | NR | NR | MUT = NF1 29%, MTOR |
| | | | | | | | | 49%, POLE 77% |
| | | | | | | | | WT = IDH1, PTEN, RB1, |
| | | | | | | | | TP53 |
| | 2401019 | PR | POS | MET | WT | NR | NR | MUT = IDH1 12%, TP53 |
| | | | | | | | | 29% |
| | | | | | | | | WT = RB1, NF1, MTOR, |
| | | | | | | | | POLE |
| | 2401020 | SD | POS | UNM | WT | NR | WT = P53 | NR |
| | 2401021 | SD | NEG | MET | WT | NR | MUT = EGFR, EGFRVIII | NR |
| | | | | | | | KI67 >50% | |
| | 2401022 | PD | NR | UNM | MUT | NR | MUT = P53 | NR |
| | | | | | | | KI67 >25% | |
| | 2401023 | SD | NEG | MET | WT | WT | MUT = GFAP, P53 (IHC), RB1, | MUT = PTEN 27%, RB1 |
| | | | | | | | BRCA1, BRCA2, MYCN, PTEN | 25%, TP53 25%, NF1 |
| | | | | | | | 89%, TP53 89% | 23%, MTOR 18%, |
| | | | | | | | WT = 1P/19Q, ATRX, BRAF, EGFR, | POLE 17% |
| | 2401024 | PD | NEG | MET | WT | WT | MUT = CDK4, EGFR, MDM2 | MUT = PTEN 58% |
| | | | | | | | KI67 HIGH | WT = IDH1, RB1, TP53, |
| | | | | | | | | NF1, MTOR, |
| | | | | | | | | POLE |
| | 2401025 | SD | NR | UNM | WT | WT | MUT = GFAP, ATM, CDKN2A, | MUT = PTEN 34%, |
| | | | | | | | PTEN, RB1, TERT, TP53 | TP53 33% |
| | | | | | | | KI67 25-30% | WT = IDH1, RB1, NF1, |
| | | | | | | | WT = EGFRVIII | MTOR, POLE |
| | 2401026 | SD | NEG | MET | WT | WT | MUT = PTEN | NR |
| | | | | | | | KI67 10-15% | |
| | | | | | | | WT = 1P/19Q, EGFR, P53 | |
| | 2401027 | SD | NEG | UNM | WT | WT | MUT = EGFR | NR |
| | | | | | | | KI67 20% | |
| | | | | | | | WT = P53 | |
| | 2401028 | PD | POS | MET | NR | NR | MUT = GFAP, P53 | NR |
| | | | | | | | KI67 60% | |
| | 2401029 | SD | POS | UNM | WT | WT | MUT = 22Q, ATRX, DDX3X, NF1, | MUT = PTEN 40%, RB1 |
| | | | | | | | PTEN, TP53 | 45%, TP53 41%, NF1 |
| | | | | | | | | 41% |
| | | | | | | | | WT = IDH1, MTOR, |
| | | | | | | | | POLE |
| | 2401030 | PD | POS | UNM | WT | WT | MUT = EGFRVIII, PTEN, TERT | NR |
| | 2401031 | SD | NR | UNM | WT | WT | MUT = CDKN2A, PTEN, EGFRVIII | WT = IDH1, PTEN, RB1, |
| | | | | | | | | TP53, NF1, MTOR, |
| | | | | | | | | POLE |
| | 2401032 | SD | NEG | UNM | WT | WT | MUT = CDKN2A, CDKN2B, | MUT = TP53 15% |
| | | | | | | | CREBBP, MYCN, PTEN, TERT, | WT = IDH1, PTEN, RB1, |
| | | | | | | | TP53 | NF1, MTOR, POLE |
| | 2401033 | SD | NEG | UNM | WT | NR | MUT = CDKN2A/B, EGFRVIII, | WT = IDH1, PTEN, RB1, |
| | | | | | | | ERRFI1, TERT | TP53, NF1, |
| | | | | | | | | MTOR, POLE |
| | 2401034 | PD | NR | UNM | WT | WT | MUT = EBFRVIII, GFAP, P53 5% | NR |
| | | | | | | | KI67 30% | |
| | | | | | | | WT = 1P/19Q | |
| | 2401035 | PD | NR | UNM | WT | WT | MUT = CDK4, CDKN2AP16INK4A, | NR |
| | | | | | | | CDKN2B, MDM2, PTEN | |
| | 2401036 | PD | NEG | UNM | WT | WT | MUT = ARID5B, CDKN2A, CDKN2B, | NR |
| | | | | | | | EGFR, NF1, SUFU, TERT 41% | |
| | 2401037 | PD | POS | UNM | WT | WT | MUT = 1P36/1Q24, ANKRD11 6%, | NR |
| | | | | | | | CDKN2AP14ARF, | |
| | | | | | | | CDKN2AP16INK4A, CDKN2B, | |
| | | | | | | | EGFR, FOXA1 39%, GATA3, | |
| | | | | | | | IKBKE, IL10, KMT2B 26%, MDM4, | |
| | | | | | | | TERT 36% | |
| | 2401038 | PD | POS | MET | WT | WT | MUT = CCND2, CDK4, EGFR, | NR |
| | | | | | | | EIF1AX, FAM58A, GLI1, MDM2, | |
| | | | | | | | SDHD, TERT | |
| | 2401039 | PR | NEG | UNM | WT | WT | MUT = CDKN2A/B, EGFRVIII, | WT = IDH1, PTEN, RB1, |
| | | | | | | | MDM4, NF1, PIK3CB, RUNX1, | TP53, NF1, MTOR, |
| | | | | | | | TERT | POLE |
| | 2401040 | PD | NEG | UNM | MUT | WT | MUT = ATRX 59%, BAP1, BLM 19%, | MUT = IDH1 30%, TP53 |
| | | | | | | | CDKN2AP14ARF, | 44%, NF1 21%, MTOR |
| | | | | | | | CDKN2AP16INK4A, CDKN2B, | 33% |
| | | | | | | | ERCC3 6%, MAP2K4 16%, MED12 | WT = PTEN, RB1, POLE |
| | | | | | | | 9%, MLH1, PBRM1, PDGFRA, | |
| | | | | | | | RAD51C 9%, SETD2, SPEN 25%, | |
| | | | | | | | TERT 25%, TGFBR2, TP53 exon5 | |
| | | | | | | | 46%, exon 7 47% | |
| | 2401041 | SD | NR | UNM | WT | NR | PDGFRA, PTEN 25%, TERT 22%, | NR |
| | | | | | | | 19% | |
| | 2401042 | PD | NEG | MET | WT | WT | MUT = CCND2, CDK4, EGFR, MET, | NR |
| | | | | | | | SOX2, TERT, TP53 | |
| | 2401043 | PD | NR | MET | WT | WT | WT = BRAF, EGFR, PTEN | NR |
| | 2401044 | SD | NR | UNM | WT | WT | MUT = glioma mutation panel | NR |
| | | | | | | | KI67 14% | |
| | | | | | | | WT = H3K27M | |
| | 2401045 | CR | NR | MET | WT | WT | WT = BRAF, TP53 | MUT = TP53 16% |
| | | | | | | | | WT = IDH1, PTEN, RB1, |
| | | | | | | | | NF1, MTOR, POLE |
| | 2401046 | SD | NEG | UNM | WT | WT | MUT = TP53, ATRX, H3F3AK27M, | MUT = TP53 77%, NF1 |
| | | | | | | | NF1, PIK3CA, RB1 | 44%, POLE 57% |
| | | | | | | | | WT = IDH1, PTEN, RB1, |
| | | | | | | | | MTOR |
| | 2401047 | PR | NEG | NR | WT | NR | MUT = H3K27M | WT = IDH1, PTEN, RB1, |
| | | | | | | | ATRX retained | TP53, NF1, MTOR, |
| | | | | | | | | POLE |
| | 2401048 | SD | NR | UNM | WT | WT | MUT = GFAP, H3K27ME, TP53, | NR |
| | | | | | | | Y236H | |
| | | | | | | | WT = H3K27M | |

¹Assay and analysis performed by Neogenomics Inc. as directed by Merck;
²Various assays used;
³Targeted NGS using NeoType Discovery Profile for Solid Tumors (NeoGenomics) and/or Novogene PM2.0.(Novogene);
Abbreviations: CR = complete response,
PD = progressive disease,
PR = partial response,
SD = stable disease;
MET = MGMT promoter methylation present/positive (methylated, hypermethylated),
UNM = MGMT promoter methylation absent/negative (unmethylated),
MUT = mutated/amplified/positive for mutation,
WT = wild type/no amplification/benign/negative for mutation,
NR = not reported/not tested

TABLE 13
Cell markers and RNA signatures

| HUGO symbols | Cell population or score | Type |
|---|---|---|
| BLK | B-cells | Cell type |
| CD19 | B-cells | Cell type |
| MS4A1 | B-cells | Cell type |
| TNFRSF17 | B-cells | Cell type |
| PTPRC | CD45 | Cell type |
| CD8A | CD8 T cells | Cell type |
| CD8B | CD8 T cells | Cell type |
| CTSW | Cytotoxic cells | Cell type |
| GNLY | Cytotoxic cells | Cell type |
| GZMA | Cytotoxic cells | Cell type |
| GZMB | Cytotoxic cells | Cell type |
| GZMH | Cytotoxic cells | Cell type |
| KLRB1 | Cytotoxic cells | Cell type |
| KLRD1 | Cytotoxic cells | Cell type |
| KLRK1 | Cytotoxic cells | Cell type |
| PRF1 | Cytotoxic cells | Cell type |
| CCL13 | Dendritic cells | Cell type |
| CD209 | Dendritic cells | Cell type |
| HSD11B1 | Dendritic cells | Cell type |
| CD244 | Exhausted CD8 | Cell type |
| EOMES | Exhausted CD8 | Cell type |
| LAG3 | Exhausted CD8 | Cell type |
| CD163 | Macrophages | Cell type |
| CD68 | Macrophages | Cell type |
| CD84 | Macrophages | Cell type |
| MS4A2 | Mast cells | Cell type |
| TPSAB1/B2 | Mast cells | Cell type |
| CSF3R | Neutrophils | Cell type |
| FCGR3A/B | Neutrophils | Cell type |
| S100A12 | Neutrophils | Cell type |
| IL21R | NK CD56dim cells | Cell type |
| KIR2DL3 | NK CD56dim cells | Cell type |
| KIR3DL1 | NK CD56dim cells | Cell type |
| KIR3DL2 | NK CD56dim cells | Cell type |
| NCR1 | NK cells | Cell type |
| CD3D | T-cells | Cell type |
| CD3E | T-cells | Cell type |
| CD3G | T-cells | Cell type |
| CD6 | T-cells | Cell type |
| SH2D1A | T-cells | Cell type |
| TBX21 | Th1 cells | Cell type |
| FOXP3 | Treg | Cell type |
| CCL2 | Chemokine | Score |
| CCL4 | Chemokine | Score |
| CCL5 | Chemokine | Score |
| CCL8 | Chemokine | Score |
| CCL18 | Chemokine | Score |
| CCL19 | Chemokine | Score |
| CCL21 | Chemokine | \|Score |
| CXCL9 | Chemokine | Score |
| CXCL10 | Chemokine | Score |
| CXCL11 | Chemokine | Score |
| CXCL13 | Chemokine | Score |
| GZMA | Cytolytic | Score |
| PRF1 | Cytolytic | Score |
| CD70 | T cell activation | Functional orientation |
| CD244 | T cell activation | Functional orientation |
| CD48 | T cell activation | Functional orientation |
| CD44 | T cell activation | Functional orientation |
| LAG3 | T cell inhibition | Functional orientation |
| TNFRSF8 | T cell inhibition | Functional orientation |
| CTLA4 | T cell inhibition | Functional orientation |
| TIGIT | T cell inhibition | Functional orientation |
| PDCD1 | T cell inhibition | Functional orientation |
| HAVCR2 | T cell inhibition | Functional orientation |
| BTLA | T cell inhibition | Functional orientation |
| ADORA2A | T cell inhibition | Functional orientation |
| TNFRSF25 | T cell inhibition | Functional orientation |
| LAIR1 | T cell inhibition | Functional orientation |
| FOXP3 | Regulatory T cells | Functional orientation |
| TNFRSF18 | Regulatory T cells | Functional orientation |
| HLA-A | Class \| MHC | Functional orientation |
| HLA-B | Class I MHC | Functional orientation |
| HLA-C | Class I MHC | Functional orientation |
| HLA-E | Class I MHC | Functional orientation |
| HLA-F | Class I MHC | Functional orientation |
| B2M | Class I MHC | Functional orientation |
| CCL2 | Myeloid cell chemotaxis | Functional orientation |
| CCL5 | Myeloid cell chemotaxis | Functional orientation |
| VEGFA | Myeloid cell chemotaxis | Functional orientation |
| CSF1 | Myeloid cell chemotaxis | Functional orientation |
| CCL2 | M1 markers | Functional orientation |
| CXCL10 | M1 markers | Functional orientation |
| GBP2 | M1 markers | Functional orientation |
| IFIT3 | M1 markers | Functional orientation |
| SLAMF7 | M1 markers | Functional orientation |
| CXCL9 | M1 markers | Functional orientation |
| CCL8 | M1 markers | Functional orientation |
| IL1B | M1 markers | Functional orientation |
| CD38 | M1 markers | Functional orientation |
| CXCL16 | M2 markers | Functional orientation |
| CXCR4 | M2 markers | Functional orientation |
| CD14 | M2 markers | Functional orientation |
| MRC1 | M2 markers | Functional orientation |
| ARG1 | M2 markers | Functional orientation |
| CCL13 | M2 markers | Functional orientation |
| MRC1 | M2 markers | Functional orientation |
| CCL2 | Tertiary lymphoid structure | Functional orientation |
| CCL4 | Tertiary lymphoid structure | Functional orientation |
| CCL5 | Tertiary lymphoid structure | Functional orientation |
| CCL8 | Tertiary lymphoid structure | Functional orientation |
| CCL18 | Tertiary lymphoid structure | Functional orientation |
| CCL19 | Tertiary lymphoid structure | Functional orientation |
| CCL21 | Tertiary lymphoid structure | Functional orientation |
| CXCL9 | Tertiary lymphoid structure | Functional orientation |
| CXCL10 | Tertiary lymphoid structure | Functional orientation |
| CXCL11 | Tertiary lymphoid structure | Functional orientation |
| CXCL13 | Tertiary lymphoid structure | Functional orientation |
| VEGFA | Angiogenic markers | Functional orientation |
| VEGFB | Angiogenic markers | Functional orientation |
| KDR | Angiogenic markers | Functional orientation |
| CXCR2 | Angiogenic markers | Functional orientation |
| HIF1A | Angiogenic markers | Functional orientation |
| ANGPT2 | Angiogenic markers | Functional orientation |
| IDO1 | IFNg signaling | Score |
| CXCL10 | IFNg signaling | Score |
| CXCL9 | IFNg signaling | Score |
| HLA-DRA | IFNg signaling | Score |
| STAT1 | IFNg signaling | Score |
| IFNG | IFNg signaling | Score |
| CD3D | IFNg downstream | Score |
| IDO1 | IFNg downstream | Score |
| CD3E | IFNg downstream | Score |
| CCL5 | IFNg downstream | Score |
| GZMK | IFNg downstream | Score |
| CD2 | IFNg downstream | Score |
| HLA-DRA | IFNg downstream | Score |
| CXCL13 | IFNg downstream | Score |
| IL2RG | IFNg downstream | Score |
| NKG7 | IFNg downstream | Score |
| HLA-E | IFNg downstream | Score |
| CXCR6 | IFNg downstream | Score |
| LAG3 | IFNg downstream | Score |
| CXCL10 | IFNg downstream | Score |
| STAT1 | IFNg downstream | Score |
| GZMB | IFNg downstream | Score |

### REFERENCES

1. Stupp, R. et al. Radiotherapy plus Concomitant and Adjuvant Temozolomide for Glioblastoma. N. Engl. J. Med. 352, 987-996 (2005).
2. Taal, W. et al. Single-agent bevacizumab or lomustine versus a combination of bevacizumab plus lomustine in patients with recurrent glioblastoma (BELOB trial): a randomised controlled phase 2 trial. Lancet. Oncol. 15, 943-53 (2014).
3. Tawbi, H. A. et al. Combined Nivolumab and Ipilimumab in Melanoma Metastatic to the Brain. N. Engl. J. Med. 379, 722-730 (2018).
4. Nayak, L. et al. Randomized Phase II and Biomarker Study of Pembrolizumab plus Bevacizumab versus Pembrolizumab Alone for Patients with Recurrent Glioblastoma. Clin. Cancer Res. 27, 1048-1057 (2021).
5. Reardon, D. A. et al. Effect of Nivolumab vs Bevacizumab in Patients with Recurrent Glioblastoma: The CheckMate 143 Phase 3 Randomized Clinical Trial. JAMA Oncol. 6, 1003-1010 (2020).
6. Ribas, A. et al. Oncolytic Virotherapy Promotes Intratumoral T Cell Infiltration and Improves Anti-PD-1 Immunotherapy. Cell 170, 1109-1119.e10 (2017).
7. Harrington, K., Freeman, D. J., Kelly, B., Harper, J. & Soria, J. C. Optimizing oncolytic virotherapy in cancer treatment. Nat. Rev. Drug Discov. 18, 689-706 (2019).
8. Gállego Pérez-Larraya, J. et al. Oncolytic DNX-2401 Virus for Pediatric Diffuse Intrinsic Pontine Glioma. N. Engl. J. Med. 386, 2471-2481 (2022).
9. Fueyo, J. et al. Preclinical characterization of the antiglioma activity of a tropism-enhanced adenovirus targeted to the retinoblastoma pathway. J. Natl. Cancer Inst. 95, 652-660 (2003).
10. Lang, F. F. et al. Phase I study of DNX-2401 (delta-24-RGD) oncolytic adenovirus: replication and immunotherapeutic effects in recurrent malignant glioma. J. Clin. Oncol. 36, 1419-1427 (2018).
11. White, K. et al. Identification, validation and biological characterization of novel Glioblastoma Tumour Microenvironment subtypes: Implications for precision immunotherapy. Ann. Oncol. (2022) doi: 10.1016/j.annonc.2022.11.008.
12. Zhao, J. et al. Immune and genomic correlates of response to anti-PD-1 immunotherapy in glioblastoma. Nat. Med. 25, 462-469 (2019).
13. Ayers, M. et al. IFN-γ-related mRNA profile predicts clinical response to PD-1 blockade. J. Clin. Invest. 127, 2930-2940 (2017).
14. Gromeier, M. et al. Very low mutation burden is a feature of inflamed recurrent glioblastomas responsive to cancer immunotherapy. Nat. Commun. 12, 352 (2021).
15. Wolchok, J. D. et al. Overall Survival with Combined Nivolumab and Ipilimumab in Advanced Melanoma. N. Engl. J. Med. 377, 1345-1356 (2017).
16. Larkin, J. et al. Five-Year Survival with Combined Nivolumab and Ipilimumab in Advanced Melanoma. N. Engl. J. Med. 381, 1535-1546 (2019).
17. Larkin, J. et al. Combined Nivolumab and Ipilimumab or Monotherapy in Untreated Melanoma. N. Engl. J. Med. 373, 23-34 (2015).
18. Borghaei, H. et al. Nivolumab versus Docetaxel in Advanced Nonsquamous Non-Small-Cell Lung Cancer. N. Engl. J. Med. 373, 1627-1639 (2015).
19. Hellmann, M. D. et al. Nivolumab plus Ipilimumab in Advanced Non-Small-Cell Lung Cancer. N. Engl. J. Med. 381, 2020-2031 (2019).
20. Kleijn, A. et al. The in vivo therapeutic efficacy of the oncolytic adenovirus Delta24-RGD is mediated by tumor-specific immunity. PLOS One 9, e97495 (2014).
21. Belcaid, Z. et al. Low-dose oncolytic adenovirus therapy overcomes tumor-induced immune suppression and sensitizes intracranial gliomas to anti-PD-1 therapy. Neuro-Oncology Adv. 2, (2020).
22. Desjardins, A. et al. Recurrent Glioblastoma Treated with Recombinant Poliovirus. N. Engl. J. Med. 379, 150-161 (2018).
23. Chiocca, E. A., Nassiri, F., Wang, J., Peruzzi, P. & Zadeh, G. Viral and other therapies for recurrent glioblastoma: is a 24-month durable response unusual? Neuro. Oncol. 21, 14-25 (2019).
24. Stupp, R. et al. NovoTTF-100A versus physician's choice chemotherapy in recurrent glioblastoma: a randomised phase III trial of a novel treatment modality. Eur. J. Cancer 48, 2192-202 (2012).
25. Wick, W. et al. Lomustine and Bevacizumab in Progressive Glioblastoma. N. Engl. J. Med. 377, 1954-1963 (2017).
26. Taal, W. et al. Single-agent bevacizumab or lomustine versus a combination of bevacizumab plus lomustine in patients with recurrent glioblastoma (BELOB trial): A randomised controlled phase 2 trial. Lancet Oncol. 15, 943-953 (2014).
27. Cloughesy, T. F. et al. Neoadjuvant anti-PD-1 immunotherapy promotes a survival benefit with intratumoral and systemic immune responses in recurrent glioblastoma. Nat. Med. 25, 477-486 (2019).
28. Samstein, R. M. et al. Tumor mutational load predicts survival after immunotherapy across multiple cancer types. Nat. Genet. 51, 202-206 (2019).
29. Todo, T. et al. Intratumoral oncolytic herpes virus G474 for residual or recurrent glioblastoma: a phase 2 trial. Nat. Med. 28, 1630-1639 (2022).
30. Wen, P. Y. et al. Updated response assessment criteria for high-grade gliomas: Response assessment in neuro-oncology working group. J. Clin. Oncol. 28, 1963-1972 (2010).
31. Wen, P. Y. et al. Response assessment in neuro-oncology clinical trials. J. Clin. Oncol. 35, 2439-2449 (2017).
32. Ellingson, B. M., Wen, P. Y. & Cloughesy, T. F. Modified Criteria for Radiographic Response Assessment in Glioblastoma Clinical Trials. Neurotherapeutics 14, 307-320 (2017).
33. Anderson, J. R., Cain, K. C. & Gelber, R. D. Analysis of survival by tumor response. J. Clin. Oncol. 1, 710-719 (1983).
34. Becht, E. et al. Estimating the population abundance of tissue-infiltrating immune and stromal cell populations using gene expression. Genome Biol. 17, 218 (2016).
35. Coppola, D. et al. Unique ectopic lymph node-like structures present in human primary colorectal carcinoma are identified by immune gene array profiling. Am. J. Pathol. 179, 37-45 (2011).
36. Rooney, M. S., Shukla, S. A., Wu, C. J., Getz, G. & Hacohen, N. Molecular and genetic properties of tumors associated with local immune cytolytic activity. Cell 160, 48-61 (2015).
37. Martinez-Valbuena, I. et al. Amylin as a potential link between type 2 diabetes and alzheimer disease. Ann. Neurol. 86, 539-551 (2019).
38. Abengozar-Muela, M. et al. Diverse immune environments in human lung tuberculosis granulomas assessed by quantitative multiplexed immunofluorescence. Mod. Pathol. 33, 2507-2519 (2020).

1. A method of treating a subject with glioma comprising:
a) determining a sample immune gene expression profile of a biopsy sample obtained from the subject comprising markers of immune infiltration by measuring the levels of mRNAs of cell type markers, immune checkpoint genes, functional orientation markers, and signature scores;
b) comparing the values of a) with a control profile comprising gene expression values from reference samples with known annotation of high (TMEhigh), medium (TMEmedium) and low (TMElow) tumor microenvironments;
c) determining the level of similarity of a) to b), and
d) administering combination therapy of an oncolytic virus and an anti-PD-1 antibody or a binding fragment thereof if there is a high level of similarity of a) to TMEmedium; a low level of similarity of a) to TMEhigh or TMElow; and/or a higher level of similarity of a) to TMEmedium than to TMEhigh or TMElow, and
not administering combination therapy of an oncolytic virus and an anti-PD-1 antibody or a binding fragment thereof if there is a low level of similarity of a) to TMEmedium; a high level of similarity of a) to TMEhigh or TMElow; and/or a higher level of similarity of a) to TMEhigh or TMElow than to TMEmedium.

2. The method of claim 1, wherein determining a higher level of similarity is indicated by a higher correlation value computed between the immune gene expression profile and the control profile, optionally wherein the correlation value is a correlation coefficient; or wherein determining a lower level of similarity is indicated by a lower correlation value computed between the sample immune gene expression profile and control profile, optionally wherein the correlation value is a correlation coefficient; optionally, wherein the correlation coefficient is a linear coefficient, optionally a Pearson correlation coefficient or a Spearman correlation coefficient.

3. (canceled)

4. (canceled)

5. The method of claim 2, wherein a high level of similarity is indicated by a Pearson correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

6. A method of treating glioma comprising administering a combination therapy of an oncolytic virus and an anti-PD-1 antibody or a binding fragment thereof to a subject that has been previously identified as having a medium tumor microenvironment,
wherein the medium tumor microenvironment has been identified by determining a similarity of an immune gene expression profile of a biopsy sample obtained pre-treatment with a control profile comprising gene expression values from reference samples with known annotation of medium (TMEmedium) tumor microenvironment, wherein the immune gene expression profile comprises mRNA levels of markers of immune infiltration; wherein the markers of immune infiltration comprises cell type markers, immune checkpoint genes, functional orientation markers, and signature scores.

7. (canceled)

8. The method of claim 1, wherein the combination therapy comprises administering the oncolytic virus intratumorally prior to administering repeated doses of the anti-PD-1 antibody or the binding fragment thereof.

9. The method of claim 1, wherein the administering of the repeated doses of the anti-PD-1 antibody or the binding fragment thereof starts about 7 days after the administering of the oncolytic virus.

10. The method of claim 1, wherein the doses of the anti-PD-1 antibody or the binding fragment thereof comprise about 200 mg for administration by intravenous infusion over about 30 minutes.

11. (canceled)

12. (canceled)

13. The method of claim 1, wherein the cell type markers comprise B-cells, CD45, CD8 T cells, Cytotoxic cells, Dendritic cells, exhausted CD8 cells, macrophages, neutrophils, NK CD56dim cells, NK cells, T-cells, Th1 cells and Treg cells.

14. The method of claim 13, wherein the B-cell markers comprise BLK, CD19, MS4A1 and TNFRSF17; the CD45 marker comprises PTPRC; the CD8 T cell markers comprise CD8A and CD8B; the cytotoxic cell markers comprise CTSW, GNLY, GZMA, GZMB, GZMH, KLRB1, KLRD1, KLRK1 and PRF1; the dendritic cell markers comprise CCL13, CD209 and HSD11B1; the exhausted CD8 cell markers comprise CD244, EOMES and LAG3; the macrophage markers comprise CD163, CD68 and CD84; the mast cell markers comprise MS4A2 and TPSAB1/B2; the neutrophil cell markers comprise CSF3R, FCGR3A/B and S100A12; the NKCD56 dim cell markers comprise IL21R, KIR2DL3, KIR3DL1 and KIR3DL2; the NK cell marker comprises NCR1; the T-cell marker comprises CD3D, CD3E, CD3G, CD6 and SH2D1A; the Th1 cell marker comprises TBX21; or the Treg cell marker comprises FOXP3.

15.-27. (canceled)

28. The method of claim 1, wherein the immune checkpoint genes comprise PD-1 (PDCD1), PD-L1 (CD274), PD-L2 (PDCD1LG2), CTLA4, TIM3, LAG3, TIGIT, B7-H3, IDO1, ICOS, NOS2, ARG2 and CXCL9.

29. The method of claim 1, wherein the functional orientation markers comprise T cell activation, T cell inhibition, Class 1 MHC, regulatory T cells, Myeloid cell chemotaxis, M1 markers, M2 markers, Tertiary lymphoid structures, angiogenic markers.

30. The method of claim 29, wherein the T cell activation markers comprise CD70, CD244, CD48 and CD44; the T cell inhibition markers comprise LAG3, TNFRSF8, CTLA4, TIGIT, PDCD1, HAVCR2, BTLA, ADORA2A, TNFRSF25 and LAIR1; the regulatory T cell markers comprise FOXP3 and TNERSF18; the Class 1 MHC markers comprise HLA-A, HLA-B, HLA-C, HLA-E, HLA-F and B2M; the myeloid cell chemotaxis markers comprise CCL2, CCL5, VEGFA and CSF1; the M1 markers comprise CCL2, CXCL10, GBP2, IFIT3, SLAMF7, CXCL9, CCL8, IL1B and CD38; the M2 markers comprise CXCL16, CXCR4, CD14, MRC1, ARG1 and CCL13; the tertiary lymphoid structure markers comprise CCL2, CCL4, CCL5, CCL8, CCL18, CCL19, CCL21, CXCL9, CXCL10, CXCL11 and CXCL13; or the angiogenic markers comprise VEGFA, VEGFB, KDR, CXCR2, HIF1A and ANGPT2.

31.-38. (canceled)

39. The method of claim 1, wherein the signature scores comprise chemokine, cytolytic, interferon gamma signaling, interferon gamma downstream signals and T cell inflamed.

40. The method of claim 39, wherein the chemokine markers comprise CCL2, CCL4, CCL5, CCL8, CCL18, CCL19, CCL21, CXCL9, CXCL10, CXCL11 and CXCL13; the cytolytic markers comprise GZMA and PRF1; the interferon signaling markers comprise IDO1, CXCL10, CXCL9, HLA-DRA, STAT1 and IFNG; the interferon gamma downstream signal markers comprise CD3D, IDO1, CD3E, CCL5, GZMK, CD2, HLA-DRA, CXCL13, IL2RG, NKG7, HLA-E, CXCR6, LAG3, CXCL10, STAT1 and GZMB; or the T cell inflamed markers comprise CCL5, CD27, CD274 [PD-L1], CD276 [B7-H3], CD8A, CMKLR1, CXCL9, CXCR6, HLA.DQA1, HLA.DRB1, HLA.E, IDO1, LAG3, NKG7, PDCD1LG2 [PD-L2], PSMB10, STAT1, and TIGIT.

41. (canceled)

42. (canceled)

43. (canceled)

44. (canceled)

45. The method of claim 1, wherein testing the sample for cell type markers, immune checkpoint genes, functional orientation markers, and signature scores comprises measuring gene expression level of ADORA2A, ANGPT2, ARG1, B2M, BLK, BTLA, CCL13, CCL18, CCL19, CCL2, CCL21, CCL4, CCL5, CCL8, CD14, CD163, CD19, CD2, CD209, CD244, CD38, CD3D, CD3E, CD3G, CD44, CD48, CD6, CD68, CD70, CD84, CD8A, CD8B, CSF1, CSF3R, CTLA4, CTSW, CXCL10, CXCL11, CXCL13, CXCL16, CXCL9, CXCR2, CXCR4, CXCR6, EOMES, FCGR3A/B, FOXP3, GBP2, GNLY, GZMA, GZMB, GZMH, GZMK, HAVCR2, HIF1A, HLA-A, HLA-B, HLA-C, HLA-DRA, HLA-E, HLA-F, HSD11B1, IDO1, IFIT3, IFNG, IL1B, IL21R, IL2RG, KDR, KIR2DL3, KIR3DL1, KIR3DL2, KLRB1, KLRD1, KLRK1, LAG3, LAIR1, MRC1, MS4A1, MS4A2, NCR1, NKG7, PDCD1, PRF1, PTPRC, S100A12, SH2D1A, SLAMF7, STAT1, TBX21, TIGIT, TNFRSF17, TNFRSF18, TNFRSF25, TNFRSF8, TPSAB1/B2, VEGFA, VEGFB.

46. The method of claim 1, wherein the glioma is glioblastoma.

47. The method of claim 1, wherein the anti-PD-1 antibody or the binding fragment thereof is pembrolizumab or a binding fragment thereof.

48. The method of claim 1, wherein the oncolytic virus is an oncolytic adenovirus.

49. The method of claim 48, wherein the oncolytic adenovirus is a conditionally replicative oncolytic adenovirus.

50. The method of claim 49, wherein the conditionally replicative oncolytic adenovirus is DNX-2401.


SEQUENCE LISTING (6 sequences)

SEQ ID NO: 1 - protein, 5 aa, Mus musculus
NYYMY

SEQ ID NO: 2 - protein, 16 aa, Mus musculus
GINPSNGGTNFNEKFK

SEQ ID NO: 3 - protein, 11 aa, Mus musculus
RDYRFDMGFDY

SEQ ID NO: 4 - protein, 15 aa, Mus musculus
RASKGVSTSGYSYLH

SEQ ID NO: 5 - protein, 7 aa, Mus musculus
LASYLES

SEQ ID NO: 6 - protein, 9 aa, Mus musculus
QHSRDLPLT